SEQUENCE LISTING (142 sequences)

SEQ ID NO: 1 - protein, 4 aa, synthetic construct
  REGION 1..4: note = Linker Sequence 1
RKAA

SEQ ID NO: 2 - skipped

SEQ ID NO: 3 - protein, 5 aa, synthetic construct
  REGION 1..5: note = Linker Sequence 3
RKARA

SEQ ID NO: 4 - protein, 6 aa, synthetic construct
  REGION 1..6: note = Linker Sequence 4
RKAAAA

SEQ ID NO: 5 - protein, 8 aa, synthetic construct
  REGION 1..8: note = Linker Sequence 5
RKAAAAAA

SEQ ID NO: 6 - protein, 8 aa, synthetic construct
  REGION 1..8: note = Linker Sequence 6
RKAASGSG

SEQ ID NO: 7 - protein, 4 aa, synthetic construct
  REGION 1..4: note = Linker Sequence 7
RKHA

SEQ ID NO: 8 - protein, 6 aa, synthetic construct
  REGION 1..6: note = Linker Sequence 8
RKHAAA

SEQ ID NO: 9 - skipped

SEQ ID NO: 10 - protein, 4 aa, synthetic construct
  REGION 1..4: note = Linker Sequence 10
GGRG

SEQ ID NO: 11 - protein, 6 aa, synthetic construct
  REGION 1..6: note = Linker Sequence 11
EARKAA

SEQ ID NO: 12 - protein, 5 aa, synthetic construct
  REGION 1..5: note = Linker Sequence 12
RKAEA

SEQ ID NO: 13-16 - skipped (4 entries)

SEQ ID NO: 17 - protein, 4 aa, synthetic construct
  REGION 1..4: note = Linker Sequence 17
RKAR

SEQ ID NO: 18 - protein, 4 aa, synthetic construct
  REGION 1..4: note = Linker Sequence 18
RKVA

SEQ ID NO: 19 - skipped

SEQ ID NO: 20 - protein, 5 aa, synthetic construct
  REGION 1..5: note = Linker Sequence 20
RKEAA

SEQ ID NO: 21 - protein, 5 aa, synthetic construct
  REGION 1..5: note = Linker Sequence 21
ERKAA

SEQ ID NO: 22 - protein, 4 aa, synthetic construct
  REGION 1..4: note = Linker Sequence 22
RKAH

SEQ ID NO: 23 - protein, 4 aa, synthetic construct
  REGION 1..4: note = Linker Sequence 23
RKAN

SEQ ID NO: 24 - protein, 6 aa, synthetic construct
  REGION 1..6: note = Linker Sequence 24
RKGGFG

SEQ ID NO: 25 - protein, 4 aa, synthetic construct
  REGION 1..4: note = Linker Sequence 25
RKGP

SEQ ID NO: 26 - protein, 5 aa, synthetic construct
  REGION 1..5: note = Linker Sequence 26
KRKAA

SEQ ID NO: 27 - protein, 5 aa, synthetic construct
  REGION 1..5: note = Linker Sequence 27
SRKAA

SEQ ID NO: 28 - protein, 6 aa, synthetic construct
  REGION 1..6: note = Linker Sequence 28
DDRKAA

SEQ ID NO: 29 - protein, 6 aa, synthetic construct
  REGION 1..6: note = Linker Sequence 29
  NON_STD 6: note = X is Citrulline
EERKVX

SEQ ID NO: 30 - protein, 4 aa, synthetic construct
  REGION 1..4: note = cleavable peptide 1
GFLG

SEQ ID NO: 31 - protein, 4 aa, synthetic construct
  REGION 1..4: note = Cleavable peptide 2
ALAL

SEQ ID NO: 32 - protein, 4 aa, synthetic construct
  REGION 1..4: note = Cleavable peptide 3
GGFG

SEQ ID NO: 33 - protein, 4 aa, synthetic construct
  REGION 1..4: note = N-terminal linker 1
AAAN

SEQ ID NO: 34-36 - skipped (3 entries)

SEQ ID NO: 37 - protein, 4 aa, synthetic construct
  REGION 1..4: note = Q-Tag 1
LLQG

SEQ ID NO: 38 - protein, 6 aa, synthetic construct
  REGION 1..6: note = Q-Tag 2
LSLSQG

SEQ ID NO: 39 - protein, 8 aa, synthetic construct
  REGION 1..8: note = Q-tag 3
GGGLLQGG

SEQ ID NO: 40 - protein, 5 aa, synthetic construct
  REGION 1..5: note = Q-Tag 4
GLLQG

SEQ ID NO: 41 - skipped

SEQ ID NO: 42 - protein, 10 aa, synthetic construct
  REGION 1..10: note = Q-Tag 6
GSPLAQSHGG

SEQ ID NO: 43 - protein, 7 aa, synthetic construct
  REGION 1..7: note = Q-Tag 7
GLLQGGG

SEQ ID NO: 44 - protein, 6 aa, synthetic construct
  REGION 1..6: note = Q-Tag 8
GLLQGG

SEQ ID NO: 45 - protein, 4 aa, synthetic construct
  REGION 1..4: note = Q-Tag 9
GLLQ

SEQ ID NO: 46 - protein, 8 aa, synthetic construct
  REGION 1..8: note = Q-Tag 10
LLQLLQGA

SEQ ID NO: 47 - protein, 5 aa, synthetic construct
  REGION 1..5: note = Q-Tag 11
LLQGA

SEQ ID NO: 48 - protein, 7 aa, synthetic construct
  REGION 1..7: note = Q-Tag 12
LLQYQGA

SEQ ID NO: 49 - protein, 6 aa, synthetic construct
  REGION 1..6: note = Q-Tag 13
LLQGSG

SEQ ID NO: 50 - protein, 6 aa, synthetic construct
  REGION 1..6: note = Q-Tag 14
LLQYQG

SEQ ID NO: 51 - protein, 7 aa, synthetic construct
  REGION 1..7: note = Q-Tag 15
LLQLLQG

SEQ ID NO: 52 - protein, 5 aa, synthetic construct
  REGION 1..5: note = Q-Tag 16
SLLQG

SEQ ID NO: 53 - protein, 5 aa, synthetic construct
  REGION 1..5: note = Q-Tag 17
LLQLQ

SEQ ID NO: 54 - protein, 6 aa, synthetic construct
  REGION 1..6: note = Q-Tag 18
LLQLLQ

SEQ ID NO: 55 - protein, 5 aa, synthetic construct
  REGION 1..5: note = Q-Tag 19
LLQGR

SEQ ID NO: 56 - protein, 8 aa, synthetic construct
  REGION 1..8: note = Q-Tag 20
EEQYASTY

SEQ ID NO: 57 - protein, 8 aa, synthetic construct
  REGION 1..8: note = Q-Tag 21
EEQYQSTY

SEQ ID NO: 58 - protein, 8 aa, synthetic construct
  REGION 1..8: note = Q-Tag 22
EEQYNSTY

SEQ ID NO: 59 - protein, 6 aa, synthetic construct
  REGION 1..6: note = Q-Tag 23
EEQYQS

SEQ ID NO: 60 - protein, 7 aa, synthetic construct
  REGION 1..7: note = Q-Tag 24
EEQYQST

SEQ ID NO: 61 - protein, 7 aa, synthetic construct
  REGION 1..7: note = Q-Tag 25
EQYQSTY

SEQ ID NO: 62 - protein, 4 aa, synthetic construct
  REGION 1..4: note = Q-Tag 26
QYQS

SEQ ID NO: 63 - protein, 6 aa, synthetic construct
  REGION 1..6: note = Q-Tag 27
QYQSTY

SEQ ID NO: 64 - protein, 5 aa, synthetic construct
  REGION 1..5: note = Q-Tag 28
YRYRQ

SEQ ID NO: 65 - protein, 5 aa, synthetic construct
  REGION 1..5: note = Q-Tag 29
DYALQ

SEQ ID NO: 66 - protein, 7 aa, synthetic construct
  REGION 1..7: note = Q-Tag 30
FGLQRPY

SEQ ID NO: 67 - protein, 10 aa, synthetic construct
  REGION 1..10: note = Q-Tag 31
EQKLISEEDL

SEQ ID NO: 68-69 - skipped (2 entries)

SEQ ID NO: 70 - protein, 5 aa, synthetic construct
  REGION 1..5: note = Q-Tag 34
LLQGG

SEQ ID NO: 71 - protein, 446 aa, synthetic construct
  REGION 1..446: note = Polatuzumab heavy chain
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 72 - protein, 218 aa, synthetic construct
  REGION 1..218: note = Polatuzumab light chain
DIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - protein, 450 aa, synthetic construct
  REGION 1..450: note = Trastuzumab heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 214 aa, synthetic construct
  REGION 1..214: note = Trastuzumab light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - protein, 447 aa, synthetic construct
  REGION 1..447: note = Enfortumab heavy chain
EVQLVESGGGLVQPGGSLRLSCAASGFTFSSYNMNWVRQAPGKGLEWVSYISSSSSTIYYADSVKGRFTISRDNAKNSLSLQMNSLRDEDTAVYYCARAYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 214 aa, synthetic construct
  REGION 1..214: note = Enfortumab light chain 1
DIQMTQSPSSVSASVGDRVTITCRASQGISGWLAWYQQKPGKAPKFLIYAASTLQSGVPSRFSGSGSGTDFTLTISSLQPEDFATYYCQQANSFPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 77 - protein, 214 aa, synthetic construct
  REGION 1..214: note = Enfortumab light chain 2
DIQMTQSPSSVSASVGDRVTITCRASQGISGWLAWYQQKPGKAPKFLIYAASTLESGVPSRFSGSGSGTDFTLTISSLQPEDFATYYCQQANSFPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 78 - protein, 335 aa, synthetic construct
  REGION 1..335: note = S. mobarensis MTG 1
FRAPDSDDRVTPPAEPLDRMPDPYRPSYGRAETVVNNYIRKWQQVYSHRDGRKQQMTEEQREWLSYGCVGVTWVNSGQYPTNRLAFASFDEDRFKNELKNGRPRSGETRAEFEGRVAKESFDEEKGFQRAREVASVMNRALENAHDESAYLDNLKKELANGNDALRNEDARSPFYSALRNTPSFKERNGGNHDPSRMKAVIYSKHFWSGQDRSSSADKRKYGDPDAFRPAPGTGLVDMSRDRNIPRSPTSPGEGFVNFDYGWFGAQTEADADKTVWTHGNHYHAPNGSLGAMHVYESKFRNWSEGYSDFDRGAYVITFIPKSWNTAPDKVKQGWP

SEQ ID NO: 79 - protein, 407 aa, synthetic construct [sequence omitted]

SEQ ID NO: 80 - protein, 395 aa, synthetic construct
  REGION 1..395: note = S. mobarensis MTG 3
MHRRIHAVGQARPPPTMARGKETKSYAETYRLTADDVANINALNESAPAASSAGPSFRAPDSDDRVTPPAEPLDRMPDPYRPSYGRAETVVNNYIRKWQQVYSHRDGRKQQMTEEQREWLSYGCVGVTWVNSGQYPTNRLAFASFDEDRFKNELKNGRPRSGETRAEFEGRVAKESFDEEKGFQRAREVASVMNRALENAHDESAYLDNLKKELANGNDALRNEDARSPFYSALRNTPSFKERNGGNHDPSRMKAVIYSKHFWSGQDRSSSADKRKYGDPDAFRSAPGTGLVDMSRDRNIPRSPTSPGEGFVNFDYGWFGAQTEADADKTVWTHGNHYHAPNGSLGCHACLTRASSATGSEGYSDFDRGEPYVVSPSPSPRMLEHRPRQGKAGLA

SEQ ID NO: 81 - protein, 395 aa, synthetic construct; identical to SEQ ID NO: 80
  REGION 1..395: note = Streptoverticillium ladakanum MTG

SEQ ID NO: 82 - protein, 4 aa, synthetic construct
  REGION 1..4: note = Linker sequence 30
RKDA

SEQ ID NO: 83 - protein, 4 aa, synthetic construct
  REGION 1..4: note = Linker sequence 31
RKNA

SEQ ID NO: 84 - protein, 6 aa, synthetic construct
  REGION 1..6: note = Linker Sequence 32
QARKAA

SEQ ID NO: 85 - protein, 5 aa, synthetic construct
  REGION 1..5: note = Linker sequence 33
RKAQA

SEQ ID NO: 86 - protein, 5 aa, synthetic construct
  REGION 1..5: note = Linker sequence 34
RKQAA

SEQ ID NO: 87 - protein, 5 aa, synthetic construct
  REGION 1..5: note = Linker sequence 35
QRKAA

SEQ ID NO: 88 - protein, 6 aa, synthetic construct
  REGION 1..6: note = Linker sequence 36
DNRKAA

SEQ ID NO: 89 - protein, 6 aa, synthetic construct
  REGION 1..6: note = Linker sequence 37
NDRKAA

SEQ ID NO: 90 - protein, 6 aa, synthetic construct
  REGION 1..6: note = Linker sequence 38
NNRKAA

SEQ ID NO: 91 - protein, 6 aa, synthetic construct
  REGION 1..6: note = Linker sequence 39
  NON_STD 6: note = X is Citrulline
EQRKVX

SEQ ID NO: 92 - protein, 6 aa, synthetic construct
  REGION 1..6: note = Linker sequence 40
  NON_STD 6: note = X is Citrulline
QERKVX

SEQ ID NO: 93 - protein, 6 aa, synthetic construct
  REGION 1..6: note = Linker sequence 41
  NON_STD 6: note = X is Citrulline
QQRKVX

SEQ ID NO: 94 - protein, 447 aa, synthetic construct
  source 1..447: note = Enfortumab heavy chain (LALA-mutation)
EVQLVESGGGLVQPGGSLRLSCAASGFTFSSYNMNWVRQAPGKGLEWVSYISSSSSTIYYADSVKGRFTISRDNAKNSLSLQMNSLRDEDTAVYYCARAYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - protein, 214 aa, synthetic construct
  source 1..214: note = Enfortumab light chain (LALA-mutation)
DIQMTQSPSSVSASVGDRVTITCRASQGISGWLAWYQQKPGKAPKFLIYAASTLNSGVPSRFSGSGSGTDFTLTISSLQPEDFATYYCQQANSFPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 96 - protein, 450 aa, synthetic construct
  source 1..450: note = m290 heavy chain
EVQLVESGGGLVQPGGSLRLSCAASGFTFSNYDMSWVRQAPGKGLEWVATISSGGSYTYYVDSVKGRFTISRDNAKNSLYLQMNSLRAEDTAVYYCARQELGSYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - protein, 219 aa, synthetic construct
  source 1..219: note = m290 light chain
DIVMTQTPLSLPVTPGEPASISCRSSQSIVHSNANTYLEWYLQKPGQSPQLLIYKVSNRFSGVPDRFSGSGSGTDFTLKISRVEAEDVGVYYCFQGSHVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 98 - protein, 449 aa, synthetic construct
  source 1..449: note = Upifitamab heavy chain
QVQLVQSGAEVVKPGASVKMSCKASGYTFTGYNIHWVKQAPGQGLEWIGAIYPGNGDTSYKQKFRGRATLTADTSTSTVYMELSSLRSEDSAVYYCARGETARATFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 214 aa, synthetic construct
  source 1..214: note = Upifitamab light chain
DIQMTQSPSSLSASVGDRVTITCSASQDIGNFLNWYQQKPGKTVKVLIYYTSSLYSGVPSRFSGSGSGTDYTLTISSLQPEDFATYYCQQYSKLPLTFGQGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 100 - protein, 449 aa, synthetic construct
  source 1..449: note = Upifitamab heavy chain (LALA-mutation)
QVQLVQSGAEVVKPGASVKMSCKASGYTFTGYNIHWVKQAPGQGLEWIGAIYPGNGDTSYKQKFRGRATLTADTSTSTVYMELSSLRSEDSAVYYCARGETARATFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 101 - protein, 214 aa, synthetic construct; identical to SEQ ID NO: 99
  source 1..214: note = Upifitamab light chain (LALA-mutation)

SEQ ID NO: 102 - protein, 121 aa, synthetic construct
  REGION 1..121: note = Trastuzumab heavy chain variable region
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSA

SEQ ID NO: 103 - protein, 107 aa, synthetic construct [sequence omitted]

SEQ ID NO: 104 - protein, 5 aa, synthetic construct
  REGION 1..5: note = Trastuzumab CDR-H1
DTYIH

SEQ ID NO: 105 - protein, 17 aa, synthetic construct
  REGION 1..17: note = Trastuzumab CDR-H2
RIYPTNGYTRYADSVKG

SEQ ID NO: 106 - protein, 11 aa, synthetic construct
  REGION 1..11: note = Trastuzumab CDR-H3
WGGDGFYAMDY

SEQ ID NO: 107 - protein, 11 aa, synthetic construct
  REGION 1..11: note = Trastuzumab CDR-L1
RASQDVNTAVA

SEQ ID NO: 108 - protein, 7 aa, synthetic construct
  REGION 1..7: note = TrastuzumabCDR-L2
SASFLYS

SEQ ID NO: 109 - protein, 9 aa, synthetic construct
  REGION 1..9: note = Trastuzumab CDR-L3
QQHYTTPPT

SEQ ID NO: 110 - protein, 118 aa, synthetic construct
  REGION 1..118: note = Polatuzumab heavy chain variable region
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSA

SEQ ID NO: 111 - protein, 111 aa, synthetic construct [sequence omitted]

SEQ ID NO: 112 - protein, 5 aa, synthetic construct
  REGION 1..5: note = Polatuzumab CDR-H1
SYWIE

SEQ ID NO: 113 - protein, 17 aa, synthetic construct
  REGION 1..17: note = Polatuzumab CDR-H2
EILPGGGDTNYNEIFKG

SEQ ID NO: 114 - protein, 8 aa, synthetic construct
  REGION 1..8: note = Polatuzumab CDR-H3
RVPIRLDY

SEQ ID NO: 115 - protein, 15 aa, synthetic construct
  REGION 1..15: note = Polatuzumab CDR-L1
KASQSVDYEGDSFLN

SEQ ID NO: 116 - protein, 7 aa, synthetic construct
  REGION 1..7: note = polatuzumab CDR-H2
AASNLES

SEQ ID NO: 117 - protein, 9 aa, synthetic construct
  REGION 1..9: note = Polatuzumab CDR-L3
QQSNEDPLT

SEQ ID NO: 118 - protein, 118 aa, synthetic construct
  REGION 1..118: note = Enfortumab heavy chain variable region
EVQLVESGGGLVQPGGSLRLSCAASGFTFSSYNMNWVRQAPGKGLEWVSYISSSSSTIYYADSVKGRFTISRDNAKNSLSLQMNSLRDEDTAVYYCARAYYYGMDVWGQGTTVTVSSA

SEQ ID NO: 119 - protein, 107 aa, synthetic construct
  REGION 1..107: note = Enfortumab light chain variable region
DIQMTQSPSSVSASVGDRVTITCRASQGISGWLAWYQQKPGKAPKFLIYAASTLQSGVPSRFSGSGSGTDFTLTISSLQPEDFATYYCQQANSFPPTFGGGTKVEIK

SEQ ID NO: 120 - protein, 5 aa, synthetic construct
  REGION 1..5: note = Enfortumab CDR-H1
SYNMN

SEQ ID NO: 121 - protein, 17 aa, synthetic construct
  REGION 1..17: note = Enfortumab CDR-H2
YISSSSSTIYYADSVKG

SEQ ID NO: 122 - protein, 8 aa, synthetic construct
  REGION 1..8: note = Enfortumab CDR_H3
AYYYGMDV

SEQ ID NO: 123 - protein, 11 aa, synthetic construct
  REGION 1..11: note = Enfortumab CDR-L1
RASQGISGWLA

SEQ ID NO: 124 - protein, 7 aa, synthetic construct
  REGION 1..7: note = Enfrotumab CDR-L2
AASTLQS

SEQ ID NO: 125 - protein, 9 aa, synthetic construct
  REGION 1..9: note = Enfortumab CDR-L3
QQANSFPPT

SEQ ID NO: 126 - protein, 121 aa, synthetic construct
  REGION 1..121: note = m290 heavy chain variable region
EVQLVESGGGLVQPGGSLRLSCAASGFTFSNYDMSWVRQAPGKGLEWVATISSGGSYTYYVDSVKGRFTISRDNAKNSLYLQMNSLRAEDTAVYYCARQELGSYYAMDYWGQGTTVTVSSA

SEQ ID NO: 127 - protein, 112 aa, synthetic construct
  REGION 1..112: note = m290 light chain variable region
DIVMTQTPLSLPVTPGEPASISCRSSQSIVHSNANTYLEWYLQKPGQSPQLLIYKVSNRFSGVPDRFSGSGSGTDFTLKISRVEAEDVGVYYCFQGSHVPYTFGGGTKVEIK

SEQ ID NO: 128 - protein, 5 aa, synthetic construct
  REGION 1..5: note = m290 CDR-H1
NYDMS

SEQ ID NO: 129 - protein, 16 aa, synthetic construct
  REGION 1..16: note = m290 CDR-H2
TISSGGSYTYYVDSVK

SEQ ID NO: 130 - protein, 11 aa, synthetic construct
  REGION 1..11: note = m290 CDR-H3
QELGSYYAMDY

SEQ ID NO: 131 - protein, 16 aa, synthetic construct
  REGION 1..16: note = m290 CDR-L1
RSSQSIVHSNANTYLE

SEQ ID NO: 132 - protein, 7 aa, synthetic construct
  REGION 1..7: note = m290 CDR-L2
KVSNRFS

SEQ ID NO: 133 - protein, 9 aa, synthetic construct
  REGION 1..9: note = m290 CDR-L3
FQGSHVPYT

SEQ ID NO: 134 - protein, 120 aa, synthetic construct
  REGION 1..120: note = Upifitamab heavy chain variable region
QVQLVQSGAEVVKPGASVKMSCKASGYTFTGYNIHWVKQAPGQGLEWIGAIYPGNGDTSYKQKFRGRATLTADTSTSTVYMELSSLRSEDSAVYYCARGETARATFAYWGQGTLVTVSSA

SEQ ID NO: 135 - protein, 107 aa, synthetic construct
  REGION 1..107: note = Upifitamab light chain variable region
DIQMTQSPSSLSASVGDRVTITCSASQDIGNFLNWYQQKPGKTVKVLIYYTSSLYSGVPSRFSGSGSGTDYTLTISSLQPEDFATYYCQQYSKLPLTFGQGTKLELK

SEQ ID NO: 136 - protein, 5 aa, synthetic construct
  REGION 1..5: note = Upifitamab CDR-H1
GYNIH

SEQ ID NO: 137 - protein, 17 aa, synthetic construct
  REGION 1..17: note = Upifitamab CDR-H2
AIYPGNGDTSYKQKFRG

SEQ ID NO: 138 - protein, 10 aa, synthetic construct
  REGION 1..10: note = upifitamab CDR-H3
GETARATFAY

SEQ ID NO: 139 - protein, 11 aa, synthetic construct
  REGION 1..11: note = Upifitimab CDR-L1
SASQDIGNFLN

SEQ ID NO: 140 - protein, 7 aa, synthetic construct
  REGION 1..7: note = Upifitimab CDR-L2
YTSSLYS

SEQ ID NO: 141 - protein, 9 aa, synthetic construct
  REGION 1..9: note = upifitimab CDR-L3
QQYSKLPLT

SEQ ID NO: 142 - protein, 7 aa, synthetic construct
  REGION 1..7: note = Enfortumab CDR-L2 variant
AASTLNS

(19) United States
(12) Patent Application Publication
(10) Pub. No.: US 2026/0151498 A1
(43) Pub. Date: Jun. 4, 2026

(54) ANTIBODY-DRUG CONJUGATES USING TWO DIFFERENT TYPES OF TOPOISOMERASE I INHIBITORS

(71) Applicant: ARARIS BIOTECH AG, Au (CH)

(72) Inventors: Romain BERTRAND, Au (CH); Isabella ATTINGER-TOLLER, Au (CH); Philipp PROBST, Au (CH); Rachael FAY, Au (CH); Lia KALLENBERGER, Au (CH); Dragan GRABULOVSKI, Au (CH); Philipp SPYCHER, Au (CH)

(21) Appl. No.: 19/269,487

(22) Filed: Jul. 15, 2025

Related U.S. Application Data
(63) Continuation of application No. PCT/EP2024/079076, filed on Oct. 15, 2024.

(30) Foreign Application Priority Data
Oct. 15, 2023 (EP) 23203677.2
Jul. 10, 2024 (EP) 24187879.2
Jul. 19, 2024 (EP) 24189861.8

(51) Int. Cl. A61K 47/68; A61P 35/00; C07K 16/28; C07K 16/32

(52) U.S. Cl. CPC A61K 47/68037; A61K 47/6849; A61K 47/6855; A61K 47/6889; A61P 35/00; C07K 16/28; C07K 16/2803; C07K 16/32

(57) ABSTRACT

Described is an antibody-drug conjugate (ADC) having the formula A-L, wherein A is an antibody or an antibody fragment and wherein L is a linker, said linker comprising: as a first payload a topoisomerase I inhibitor which is cell-permeable, preferably a camptothecin cytotoxic molecule which is cell-permeable; and as a second payload a topoisomerase I inhibitor which is not cell-permeable, preferably a camptothecin cytotoxic molecule which is not cell-permeable. Moreover, described is a pharmaceutical composition comprising said ADC and at least one pharmaceutically acceptable ingredient. Further, described is an ADC for use in a method of treating a patient suffering from, being at risk of developing, and/or being diagnosed for a neoplastic disease.

### RELATED APPLICATIONS

This application is a continuation of International Patent Application No. PCT/EP2024/079076, filed Oct. 15, 2024, which claims priority to European Patent Application Nos. 24189861.8, filed Jul. 19, 2024; 24187879.2, filed Jul. 10, 2024; and 23203677.2, filed Oct. 15, 2023, the entire disclosures of which are hereby incorporated herein by reference.

### SEQUENCE LISTING

The instant application contains a Sequence Listing which has been submitted electronically in XML format and is hereby incorporated by reference in its entirety. Said XML file, created on Jun. 17, 2025, is named 766966_ARA9-010PCCON_ST26.xml and is 170,873 bytes in size.

The present invention relates to an antibody-drug conjugate (ADC) having the formula A-L, wherein A is an antibody or an antibody fragment and wherein L is a linker, said linker comprising: as a first payload a topoisomerase I inhibitor which is cell-permeable, preferably a camptothecin cytotoxic molecule which is cell-permeable; and as a second payload a topoisomerase I inhibitor which is not cell-permeable, preferably a camptothecin cytotoxic molecule which is not cell-permeable. Moreover, the present invention relates to a pharmaceutical composition comprising said ADC and at least one pharmaceutically acceptable ingredient. Further, the present invention relates to said ADC for use in a method of treating a patient suffering from, being at risk of developing, and/or being diagnosed for a neoplastic disease.

DNA topoisomerases are essential enzymes that stabilize DNA supercoiling and resolve entanglements. Topoisomerase inhibitors have been widely used as anti-cancer drugs for the past 20 years. Due to their selectivity as topoisomerase I (TOP1) inhibitors that trap TOP1 cleavage complexes, camptothecin and its derivatives are promising anti-cancer drugs. To increase accumulation of TOP1 inhibitors in cancer cells through the targeting of tumors, TOP1 inhibitor antibody-drug conjugates (TOP1-ADC) have been developed and marketed. Some TOP1-ADCs have shown enhanced therapeutic efficacy compared to prototypical anti-cancer ADCs (Han S. et al, 2022, Pharmaceutics, 14, 1707).

One of the most prominent examples of TOP1-ADCs is DS-8201a, a HER2 targeting ADC with a novel DNA topoisomerase I inhibitor that demonstrated impressive antitumoral activity in a broad selection of HER2 positive models and favorable safety profiles (Ogitani et al. 2016, Clin Cancer Res; 22(20)), leading to its approval for several types of HER2 positive breast cancer types, non-small cell lung cancers and gastric/gastroesophageal junction adenocarcinomas (see also prescribing information for Enhertu®). Strikingly, DS-8201a showed a better antitumoral activity compared to another HER2 targeting ADC (T-DM1 or trastuzumab emtansine), and it could be shown that DS-8201a was even effective in patients who were refractory, or resistant to trastuzumab emtansine (André F. et al, 2023, The Lancet, 401, 10390, pp. 1773-1785). Moreover, DS-8201a, that has a drug-to-antibody ratio (DAR) of about 8, is still effective on tumors with low HER2 level, whereas T-DM1 (DAR of 3.5) did not have any effect on these tumors (Ogitani et al. 2016, Clin Cancer Res; 22(20)). The authors suggested that DS-8201a enables the delivery of more cytotoxic payload to HER2 moderate/low-expressing tumor cells than a lower DAR ADC, therefore, T-DM1 was not effective in these models, and the lower DAR of T-DM1 (DAR 3.5) than DS-8201a was considered to be one of the reasons. Furthermore, Ogitani et al showed in another study (Ogitani et al., 2016, Cancer Sci, 107,7, pp. 1039-1046) that the so-called “bystander killing” is another differentiation between DS-8201a and T-DM1, as solid tumors that have a heterogeneous target expression, i.e., some tumor cells do not express the tumor antigen and therefore cannot be reached by an ADC, are more difficult to treat compared to for example hematological malignancies. The authors confirmed that the payload of DS-8201a, Dxd, was highly membrane-permeable whereas that of T-DM1, Lys-SMCC-DM1, had a low level of permeability. For example, under a coculture condition of HER2-positive KPL-4 cells and negative MDA-MB-468 cells in vitro, DS-8201a killed both cells, whereasT-DM1 did not. In vivo evaluation was carried out using mice inoculated with a mixture of HER2-positive NCI-N87 cells and HER2-negative MDA-MB-468-Luc cells by using an in vivo imaging system. In vivo, DS-8201a reduced the luciferase signal of the mice, indicating suppression of the MDA-MB-468-Luc population; however, T-DM1 did not. Furthermore, it was confirmed that DS-8201a was not effective against MDA-MB-468-Luc tumors inoculated at the opposite side of the NCI-N87 tumor, suggesting that the bystander killing effect of DS-8201a is observed only in cells neighboring HER2-positive cells, indicating low concern in terms of systemic toxicity. These results indicated that DS-8201a has a potent bystander effect due to a highly membrane-permeable payload and is beneficial in treating tumors with HER2 heterogeneity that are unresponsive to T-DM1.

In summary, prior art teaches that for the development of potent and effective TOP1-ADC it is important to have a
High drug-to antibody ratio (DAR) of ideally 8; and
Cytotoxic topoisomerase I inhibitor as payload that is highly cell-permeable to exert bystander killing.

Although the prior art already provides efficient anti-cancer drugs as outlined above, there is still a need to provide further means for the effective treatment of cancer and, more specifically, there is still a need for improvements, in particular, as regards a further improvement of anti-tumor effects of anti-cancer drugs.

The present invention is, at least in part, based on the surprising finding that unexpectedly, against the above prior art knowledge, an ADC generated using the same HER-2 targeting antibody as used in DS-8201a (=Enhertu®), having a DAR of only 4, comprising 2 topoisomerase I inhibitors that are cell-permeable and 2 topoisomerase I inhibitors that are not cell-permeable, showed a significantly improved anti-tumor effect compared to DS-8201a.

More specifically, as exemplified in more detail in the Example-section, below, an ADC was generated having 2 exatecans per antibody (exemplary payload that shows cell permeation), and 2 Gly-exatecans that have a glycine residue linked to exatecan (exemplary payload that shows no cell permeation).

Accordingly, the present invention is, at least in part, based on the surprising finding that a combination of two different topoisomerase I inhibitors has a synergistic effect over the gold standard DS-8201a, primarily because Gly-exatecan is able to kill target-positive cells very efficiently (payload accumulates within cell and does not diffuse), whereas additionally the cytotoxic payload with bystander activity can kill not only the targeted cancer cells but also neighboring cells that might not express the target, addressing tumor heterogeneity and reducing the risk of relapse.

In view of the prior art, taking into account these beneficial and surprising effects, the technical problem underlying the present invention is the provision of anti-cancer drugs having improved anti-tumor effects.

The technical problem is solved by provision of the embodiments provided herein and as characterized in the claims.

The present invention thus relates to:
an antibody-drug conjugate (ADC) having the formula A-L, wherein A is an antibody or an antibody fragment and wherein L is a linker, said linker comprising:
as a first payload a topoisomerase I inhibitor which is cell-permeable, preferably a camptothecin cytotoxic molecule which is cell-permeable; and
as a second payload a topoisomerase I inhibitor which is not cell-permeable, preferably a camptothecin cytotoxic molecule which is not cell-permeable.

Accordingly, the present invention relates to an antibody drug conjugate (ADC) which comprises three major components, i.e.,
an antibody,
a linker, and
a topoisomerase I inhibitor as a first and second payload, wherein the topoisomerase I inhibitor of the first payload is cell-permeable and wherein the topoisomerase I inhibitor of the second payload is not cell-permeable.

In a particular embodiment, the present invention relates to an antibody drug conjugate (ADC) which comprises three major components, i.e.,
an antibody,
a linker, and
a camptothecin cytotoxic molecule as a first and second payload, respectively, wherein the camptothecin cytotoxic molecule of the first payload is cell-permeable and wherein the camptothecin cytotoxic molecule of the second payload is not cell-permeable.

These three major components are described in more detail in the following.

The term “antibody-drug conjugate” (ADC) refers to a targeted therapy that combines an antibody specific to a particular antigen with a cytotoxic drug (payload) through a linker. The antibody directs the payload to cells expressing the target antigen, such as cancer cells, allowing for precise delivery of the cytotoxic agent to the desired cells, thereby minimizing damage to healthy tissues. In the present application, an antibody-drug conjugate is also referred to as an “antibody-payload conjugate.”

The term “antibody” herein is used in the broadest sense and specifically covers monoclonal antibodies, polyclonal antibodies, multispecific antibodies (e.g., bispecific antibodies) formed from at least two intact antibodies, and antibody fragments so long as they exhibit the desired biological activity. The terms “antibody” and “antibodies” broadly encompass naturally occurring forms of antibodies (e.g., IgG, IgA, IgM, IgE).

The antibody is preferably a monoclonal antibody. The antibody can be of human origin, but likewise from mouse, rat, goat, donkey, hamster, or rabbit. In case the conjugate is for therapy, a murine or rabbit antibody may optionally be chimeric or humanized.

The antibody may also be bispecific (e.g., DVD-IgG, crossMab, appended IgG-HC fusion) or biparatopic. See Brinkmann and Kontermann; Bispecific antibodies; Drug Discov Today; 2015; 20(7); p.838-47, for an overview.

The term “antibody” further encompasses antigen-binding fragments of antibodies. The term “antibody fragment”, as used herein, refers to a portion of an antibody molecule that retains the ability to specifically bind to an antigen. These fragments are derived from full-length antibodies and include, but are not limited to, Fab (Fragment antigen-binding), F(ab′)2, scFv (single-chain variable fragment), dsFv (disulfide-stabilized Fv), Fab′, diabody, nanobody (VHH or single-domain antibody), and domain antibodies (dAbs). Antibody fragments are engineered to maintain the antigen-binding function while being smaller and more versatile than full-length antibodies, making them particularly useful for therapeutic, diagnostic, and research applications due to their enhanced tissue penetration, reduced immunogenicity, and ease of production and manipulation.

Preferably, the linker according to the invention, more preferably the peptide linker according to the invention, is conjugated to glutamine residue 295 (Q295) in the C_{H}2 domain of an IgG antibody. Thus, it is preferred herein that the antibody or antibody fragment of the invention comprises a C_{H}2 domain.

Fragments or recombinant variants of antibodies comprising the CH2 domain may be, for example,
antibody formats comprising mere heavy chain domains (shark antibodies/IgNAR (VH-CH1-CH2-CH3-CH4-CH5)2 or camelid antibodies/hclgG (VH-CH2-CH3)2)
scFv-Fc (VH-VL-CH2-CH3)2
Fc fusion peptides, comprising an Fc domain and one or more receptor domains.

In a particular embodiment, the invention relates to the antibody-payload conjugate according to the invention, wherein the antibody is an IgG antibody.

By “IgG” as used herein is meant a polypeptide belonging to the class of antibodies that are substantially encoded by a recognized immunoglobulin gamma gene. In humans, IgG comprises the subclasses or isotypes IgG1, IgG2, IgG3, and IgG4. In mice, IgG comprises IgG1, IgG2a, IgG2b, IgG3. Full-length IgGs consist of two identical pairs of two immunoglobulin chains, each pair having one light and one heavy chain, each light chain comprising immunoglobulin domains VL and CL, and each heavy chain comprising immunoglobulin domains VH, Cγ1 (also called C_{H}1), Cγ2 (also called C_{H}2), and Oγ3 (also called C_{H}3). In the context of human IgG1, “C_{H}1” refers to positions 118-215, C_{H}2 domain refers to positions 231-340 and C_{H}3 domain refers to positions 341-447 according to the EU index as in Kabat. IgG1 also comprises a hinge domain which refers to positions 216-230 in the case of IgG1.

In a preferred embodiment, the antibody is an IgG1 or IgG4 antibody. In a particularly preferred embodiment, the antibody is a human IgG1 antibody.

More preferred embodiments of the antibody in terms of the present invention are outlined further below.

In a particular preferred embodiment, the invention relates to the antibody-payload conjugate according to the invention, wherein the antibody is an antibody that comprises a mutation that reduces, ablates or eliminates FcγR binding.

Mutations in an antibody, preferably in the constant region of the heavy chain of an antibody, that reduce, ablate or eliminate FcγR binding are known in the art and are contemplated in the context of the present invention.

Without being bound to theory, a combination of the mutations Leu234Ala and Leu235Ala (according to Kabat positions numbering) are known to eliminate binding to FcγRI, IIa, and IIIa for both IgG1 and IgG4. This combination of the mutations Leu234Ala and Leu235Ala in the constant region of the heavy chain of an antibody is commonly called “LALA mutations”. This type of mutation is commonly known in the art as a modification in an antibody's Fc region in order to reduce the antibody's effector function. The article of Liu et al., Antibodies 9(4):64 (2020)) reviews current technologies of Fc-engineering for modulated effector functions of antibodies, in particular, for improving the performance of antibodies in cancer treatment while the above “LALA mutations” are also described therein.

Accordingly, in a preferred embodiment, the invention relates to the antibody-payload conjugate according to the invention, wherein the antibody is an antibody, preferably, an IgG antibody comprising at Kabat position 234 an A and/or at Kabat position 235 an A.

Thus, in a preferred embodiment, the invention relates to the antibody-payload conjugate according to the invention, wherein the antibody is an antibody, preferably, an IgG1 or an IgG4 antibody comprising at Kabat position 234 an A and/or at Kabat position 235 an A.

More preferred embodiments of the antibody in terms of the present invention comprising at Kabat position 234 an A and/or at Kabat position 235 an A are outlined further below.

The linker, preferably, the peptide linker described in more detail further below, comprises a cytotoxic molecule, i.e., a toxin as a payload which is a topoisomerase I inhibitor, preferably a camptothecin.

The terms “toxin” and “cytotoxic molecule” are known in the art and generally relate to any compound that is poisonous to a cell or organism. The toxin/cytotoxic molecule may be produced by a cell or an organism. However, a toxin/cytotoxic molecule may also be a chemical derivative or analog of a toxin that is produced by a cell or an organism. In general, toxins/cytotoxic molecules are understood to be, without limitation, small molecules, peptides, or proteins. Specific examples are neurotoxins, necrotoxins, hemotoxins and cytotoxins.

In accordance with the present invention, the toxin/cytotoxic molecule is a toxin/cytotoxic molecule that is used in the treatment of neoplastic diseases. That is, the toxin/cytotoxic molecule is conjugated to an antibody delivered to or into a malignant cell due to the target specificity of the antibody.

In the present invention, the toxin/cytotoxic molecule is a topoisomerase I inhibitor, preferably a camptothecin. A “topoisomerase I inhibitor” as used herein is a molecule that functions as a topoisomerase I inhibitor and may comprise camptothecins and non-camptothecins. Examples of non-camptothecin topoisomerase I inhibitors are indolocarbazoles, dibenzonaphthyridines and indenoisoquinolones. The term “camptothecin” as used herein is intended to mean a camptothecin or camptothecin derivative that functions as a topoisomerase I inhibitor. Exemplary camptothecins include, for example, topotecan, exatecan, deruxtecan, irinotecan, belotecan, rubitecan, gimatecan, silatecan, cositecan, DX-8951f, SN38, BN 80915, lurtotecan, AZ0132, CPT1, CPT2, Dxd(1), Dxd(2), 9-nitrocamptothecin and aminocamptothecin. A variety of camptothecins have been described, including camptothecins used to treat human cancer patients. Several camptothecins are described, for example, in Kehrer et al., Anticancer Drugs, 12 (2): 89-105, (2001) or Li et al., ACS Med. Chem. Lett. 2019, 10, 10, 1386-1392). In certain embodiments the camptothecin is the exatecan derivative shown as compound 10 in Li et al., ACS Med. Chem. Lett. 2019, 10, 10, 1386-1392). In certain embodiments, the camptothecin derivative is a glycinated exatecan (G-Exa; see e.g. FIG. 4).

In the present invention, a camptothecin cytotoxic molecule is used as a first and second payload, respectively, wherein the camptothecin cytotoxic molecule of the first payload is cell-permeable and wherein the camptothecin cytotoxic molecule of the second payload is not cell-permeable.

Methods are known in the art to render a molecule (in the present case the camptothecin cytotoxic molecule) cell-permeable or not cell-permeable.

Without being bound by theory and as, e.g., exemplified in the Example-section, below, the camptothecin cytotoxic molecule of the second payload is rendered to be not cell-permeable by adding covalently a glycine residue to the primary amine on the F ring of the camptothecin cytotoxic molecule.

In a preferred embodiment, the glycinated camptothecin is a glycinated exatcan having the following structure:

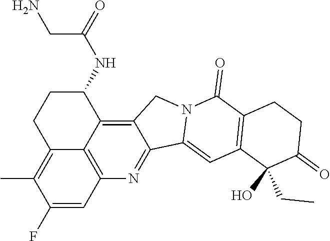 [SMILES: CC[C@@]1(O)C(=O)CCc2c1cc1n(c2=O)Cc2c-1nc1cc(F)c(C)c3c1c2[C@@H](NC(=O)CN)CC3]

The skilled person is aware of additional, common strategies to reduce the cell-permeability of a drug: small uncharged lipophilic molecules are more likely to permeate across cell barriers than those which are charged and hydrophilic, therefore, modifications of topoisomerase 1 inhibitors which will increase the overall polarity and hydrophilicity (by adding charge through highly ionizable groups such as primary, secondary or tertiary amine, carboxylate, phosphonate, sulfonate, nitro, and others) will reduce the cell-permeability.

Accordingly, in a particular embodiment, the invention relates to the antibody-payload conjugate according to the invention, wherein the second payload has been modified to reduce its cell permeability. Topoisomerase 1 inhibitors are preferably modified to reduce their cell permeability by covalently linking the topoisomerase 1 inhibitor to molecules that will increase the overall polarity and/or hydrophilicity. In a preferred embodiment, the polarity and/or hydrophilicity of the topoisomerase 1 inhibitors may be increased by covalently linking the topoisomerase 1 inhibitors to an amino acid residue, preferably a glycine residue.

Moreover, the capability of a molecule (in the present case the topoisomerase I inhibitor conjugated to the antibody) to be cell-permeable or not cell-permeable can be measured/determined by methods known in the art and as described herein. Without being bound by theory, this capability can be measured/determined as follows:

The ADC of interest can be incubated under coculture conditions essentially as described by Ogitani et al. (Ogitani et al., 2016, Cancer Sci, 107,7, pp. 1039-1046): Antigen-positive cells and antigen negative cells are cocultured in vitro, and upon incubation with the ADC cell viability is measured. If the ADC is able to kill preferentially the target positive cells, then the payload does not permeate to the adjacent antigen negative cells (“no bystander activity”), whereas if the ADC of interest kills both cells then the payload is cell permeable (“bystander activity”).

A payload of an ADC is considered “cell-permeable”, within the meaning of the present invention, if ADC incubation results in efficient killing of target positive cells and at least 70%, preferably 80%, more preferably 90% and most preferably more than 95% of the target negative cells under said coculture conditions.

A payload of an ADC is considered “not cell-permeable”, within the meaning of the present invention, if ADC incubation results in efficient killing of target positive cells and less than 30%, preferably less than 20%, preferably less than 10% and most preferably less than 5% of the target negative cells under said coculture conditions.

In a particular embodiment, the first payload, i.e., the cell-permeable topoisomerase 1 inhibitor is exatecan:

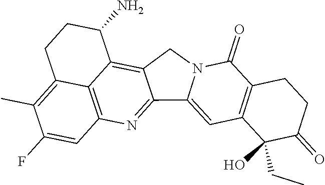 [SMILES: CC[C@@]1(O)C(=O)CCc2c1cc1n(c2=O)Cc2c-1nc1cc(F)c(C)c3c1c2[C@@H](N)CC3]

In such embodiments, the exatecan is preferably attached via the primary amine on the F ring to a self-immolative moiety comprised in the linker to allow for the release of the fully active, chemically unmodified payload.

The second payload is preferably a modified exatecan, wherein the exatecan is linked via the primary amine on the F ring to a molecule that will increase the overall polarity and/or hydrophilicity, preferably to an amino acid residue, more preferably to a glycine residue.

As mentioned, the invention relates to an antibody-payload conjugate comprising an antibody conjugated to a linker.

A linker generally refers to a molecule that connects two or more parts of a conjugate or construct. Linker are often also referred to as spacers.

It is to be understood that the present invention is based on the surprising finding that the combination of two different payloads—a cell-permeable topoisomerase I inhibitor and a cell-impermeable topoisomerase I inhibitor—results in potent anti-tumor activity. Therefore, the present invention is not limited to a specific conjugation strategy and any possible linker can be used in accordance with the present invention.

That is, the linker according to the invention may be conjugated to an antibody using various strategies, including unspecific conjugation methods such as lysine conjugation involving NHS esters or isothiocyanates to react with amine groups on lysine residues, and cysteine conjugation using maleimides, iodoacetamides, or disulfide rebridging reagents to target thiol groups of cysteine residues. Additionally, site-specific conjugation strategies may be employed, such as enzymatic conjugation (e.g., transglutaminase, sortase), unnatural amino acid incorporation, or click chemistry for precise and homogeneous ADCs.

In a preferred embodiment, the linker is a peptide linker.

A “peptide linker”, within the meaning of the present invention, is a molecule comprising at least two amino acid residues, wherein the two amino acid residues are coupled via a peptide bond. The peptide linker may be modified with one or more reactive groups to allow conjugation of the peptide linker to an antibody. For example, the peptide linker may be functionalized with a maleimide to allow conjugation to cysteine residues of an antibody.

However, it is envisioned that the peptide linker is suitable as a substrate for a microbial transglutaminase. In particular, it is envisioned that the peptide linker is suitable for conjugation to a glutamine residue comprised in an antibody. For that, the peptide linker according to the invention has to comprise at least one amino acid residue comprising a primary amine.

Thus, in a particular embodiment, in the peptide linker according to the invention the primary amine comprised in the amino acid residue is
a) a primary amine in a side chain of a lysine, a lysine derivative or a lysine mimetic; or
b) a primary amine comprised in an N-terminal amino acid residue having the structure NH2-(Y)—COOH.

That is, in a preferred embodiment, the amino acid residue comprising the primary amine is a lysine residue. In such embodiments, the peptide linker comprises a peptide moiety comprising at least one lysine residue.

However, the linker in accordance with the invention may also comprise a lysine mimetic or a lysine derivative, provided that the lysine mimetic or lysine derivative comprises a free primary amine in the amino acid side chain.

In certain embodiments, the amino acid residue comprising the primary amine may be a lysine mimetic. The term “lysine mimetic”, as used herein, refers to a compound that has a structure different from lysine, but that has similar characteristics as lysine and may thus be used to replace lysine in a peptide or protein without significantly altering the function and/or structure of said peptide or protein. In certain embodiments, a lysine mimetic may differ from lysine in the length or composition of the aliphatic chain that connects the primary amine and the α-carbon atom. Thus, in certain embodiments, the lysine mimetic may be ornithine, homolysine or 2,7-diaminoheptanoic acid. In certain embodiments, the lysine mimetic may be a beta-amino acid, such as beta-homolysine.

In certain embodiments, the amino acid residue comprising the primary amine may be a lysine derivative. The term “lysine derivative”, as used herein, refers to a lysine or lysine mimetic, wherein one or more functional groups comprised in the lysine or lysine mimetic is (are) modified or substituted. It is preferred that the amino group in the side chain of the lysine derivative is unmodified, such that is available for conjugation to a glutamine residue in a protein. Thus, the “lysine derivative” comprised in the peptide linker in accordance with the present invention preferably comprises a modified or substituted α-amino and/or α-carboxyl group.

In certain embodiments, the primary amine comprised in the amino acid residue may be a primary amine comprised in an N-terminal amino acid residue having the structure NH₂—(Y)—COOH.

In certain embodiments, the primary amine may be the α-amino group of an α-amino acid. The α-amino acid may be any proteinogenic α-amino acid, including alanine, arginine, asparagine, aspartic acid, cysteine, glutamic acid, glutamine, glycine, histidine, isoleucine, leucine, lysine, methionine, phenylalanine, serine, threonine, tryptophan, tyrosine and valine.

In a particularly preferred embodiment, the primary amine may be the α-amino group of a glycine residue. In such embodiments, it is preferred that the glycine residue is the N-terminal amino acid residue of the peptide linker, such that the α-amino group is available for conjugation to a glutamine residue via a microbial transglutaminase.

The amino acid comprising the primary amine may be a non-canonical or a synthetic amino acid. A “non-canonical amino acid”, as used herein, may be any amino acid that is not part of the set of proteinogenic amino acids, but that can be obtained from a natural source. However, it has to be noted that some non-canonical amino acids may also be found in naturally occurring peptides and/or proteins. A “synthetic amino acid”, as used herein, may be any molecule that falls under the general definition of an amino acid (NH₂—(Y)—COOH), i.e., that comprises an amino group and a carboxyl group, but that is not found in nature. Thus, non-natural amino acids are preferably obtained by chemical synthesis. It is to be understood that the differentiation between a non-canonical amino acid and a synthetic amino acid may be uncertain in some instances. For example, an amino acid that is defined as a synthetic amino acid may be, at a later time point, identified in nature and thus reclassified as a non-canonical amino acid. The non-canonical or synthetic amino acid may be an α-, β-, γ-, δ-, or ε-amino acid.

In certain embodiments, the amino acid comprising the primary amine may have the structure NH₂—(Y)—COOH.

In certain embodiments, the moiety Y may comprise a carbon comprising framework of 1 to 200 atoms, optionally a carbon comprising framework of at least 10 atoms, e.g. 10 to 100 atoms or 20 to 100 atoms, substituted at one or more atoms, optionally wherein the carbon comprising framework is a linear hydrocarbon or comprises a cyclic group, a symmetrically or asymmetrically branched hydrocarbon, monosaccharide, disaccharide, linear or branched oligosaccharide (asymmetrically branched or symmetrically branched), other natural linear or branched oligomers (asymmetrically branched or symmetrically branched), or more generally any dimer, trimer, or higher oligomer (linear, asymmetrically branched or symmetrically branched) resulting from any chain-growth or step-growth polymerization process.

Y may further be any straight, branched and/or cyclic C₂₋₃₀ alkyl, C₂₋₃₀ alkenyl, C₂₋₃₀ alkynyl, C₂₋₃₀ heteroalkyl, C₂₋₃₀ heteroalkenyl, C₂₋₃₀ heteroalkynyl, optionally wherein one or more homocyclic aromatic compound radical or heterocyclic compound radical may be inserted; notably, any straight or branched C₂₋₅ alkyl, C₅₋₁₀ alkyl, Cn-20 alkyl, —O—C₁₋₅ alkyl, —O—C₅₋₁₀ alkyl, —O—C₁₁₋₂₀ alkyl, or (CH₂—CH₂—O—)₁₋₂₄ or (CH₂)ₓ₁—(CH₂—O—CH₂)₁₋₂₄—(CH₂)ₓ₂— group, wherein x1 and x2 are independently an integer selected among the range of 0 to 20, an amino acid, an oligopeptide, glycan, sulfate, phosphate, or carboxylate. In some embodiments, Y may comprise a C₂₋₆ alkyl group.

In a particular embodiment, in the peptide linker according to the invention, Y is —(R₂C)ₙ— and wherein n is an integer ranging from 1 to 20, from 1 to 15, from 1 to 10.

That is, Y may have the structure

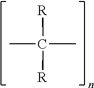

In certain embodiments, Y may be a substituted or unsubstituted alkyl or alkenyl chain. When Y is a substituted or unsubstituted alkenyl chain, it is to be understood that at least two R moieties attached to consecutive carbon molecules have to be absent.

The term “substituted alkyl”, as used herein, generally refers to an alkyl group with an additional group or groups attached to any carbon of the alkyl group. That is, the substituted alkyl may comprise the structure —(R₂C)ₙ—, wherein each R may independently be a hydrogen or a functional group such as an alkyl, lower alkyl, aryl, acyl, halogen, alkylhalo, hydroxy, amino, alkoxy, alkylamino, acylamino, acyloxy, aryloxy, aryloxyalkyl, mercapto, both saturated and unsaturated cyclic hydrocarbon, heterocycle, and other organic group.

In certain embodiments, the amino acid comprising the primary amine may have the structure NH₂—(Y)—COOH, wherein Y is —(R₂C)ₙ— and wherein n is 1, 2, 3, 4, 5, 6, 7, 8, 9, 10, 11, 12, 13, 14, 15, 16, 17, 18, 19 or 20. In certain embodiments, at least 1, 2, 3, 4 or 5 moieties R comprised in the structure —(R₂C)ₙ— may be a functional group such as an alkyl, lower alkyl, aryl, acyl, halogen, alkylhalo, hydroxy, amino, alkoxy, alkylamino, acylamino, acyloxy, aryloxy, aryloxyalkyl, mercapto, both saturated and unsaturated cyclic hydrocarbon, heterocycle, and other organic group.

In a particular embodiment, in the peptide linker according to the invention, the at least one R moiety of each —(R₂C)— monomer is hydrogen.

That is, in certain embodiments, one R moiety of each —(R₂C)— monomer may be a hydrogen, while the other R moiety may be a functional group such as an alkyl, lower alkyl, aryl, acyl, halogen, alkylhalo, hydroxy, amino, alkoxy, alkylamino, acylamino, acyloxy, aryloxy, aryloxyalkyl, mercapto, both saturated and unsaturated cyclic hydrocarbon, heterocycle, and other organic group. Alternatively, one R moiety of each —(R₂C)— monomer may be hydrogen and the other R moiety may be absent (in case of alkenes). In certain embodiments, some —(R₂C)— monomers comprised in a moiety Y may comprise two hydrogen substituents and some —(R₂C)— monomers comprised in the same moiety Y may comprise one hydrogen substituent and one substituent R as defined herein.

In a particular embodiment, in the peptide linker according to the invention, both R moieties of each —(R₂C)— monomer are hydrogen.

In certain embodiments the structure —(R₂C)ₙ— may be an unsubstituted alkyl chain wherein all moieties R comprised in the structure —(R₂C)ₙ— are hydrogen atoms. That is, in certain embodiments, the structure —(R₂C)ₙ— may be a methyl, ethyl, propyl, butyl, pentyl, hexyl, heptyl, octyl, nonyl, decyl, undecyl, dodecyl, tridecyl, tetradecyl, pentadecyl, hexadecyl, heptadecyl or octadecyl group.

That is, in certain embodiments, the amino acid comprising the primary amine may have the structure NH₂—(Y)—COOH, wherein Y is —(CH₂)ₙ— and wherein n is an integer from 1 to 20. In certain embodiments, the amino acid comprising the primary amine may have the structure NH₂—(Y)—COOH, wherein Y is —(CH₂)ₙ— and wherein n is an integer from 1 to 15. in a certain embodiments, the amino acid comprising the primary amine may have the structure NH₂—(Y)—COOH, wherein Y is —(CH₂)ₙ— and wherein n is an integer from 1 to 10. in certain embodiments, the amino acid comprising the primary amine may have the structure NH₂—(Y)—COOH, wherein Y is —(CH₂)ₙ— and wherein n is an integer from 1 to 9. in certain embodiments, the amino acid comprising the primary amine may have the structure NH₂—(Y)—COOH, wherein Y is —(CH₂)ₙ— and wherein n is an integer from 1 to 8. in certain embodiments, the amino acid comprising the primary amine may have the structure NH₂—(Y)—COOH, wherein Y is —(CH₂)ₙ— and wherein n is an integer from 1 to 7. in certain embodiments, the amino acid comprising the primary amine may have the structure NH₂—(Y)—COOH, wherein Y is —(CH₂)ₙ— and wherein n is an integer from 1 to 6.

In certain embodiments, Y may have the structure —(CH₂)ₙ—, wherein n is 1. That is, in certain embodiments, the amino acid comprising the primary amine may be glycine.

In certain embodiments, Y may have the structure —(CH₂)ₙ—, wherein n is 2. That is, in certain embodiments, the amino acid comprising the primary amine may be β-alanine.

In certain embodiments, Y may have the structure —(CH₂)ₙ—, wherein n is 3. That is, in certain embodiments, the amino acid comprising the primary amine may be 4-aminobutyric acid.

In certain embodiments, Y may have the structure —(CH₂)ₙ—, wherein n is 4. That is, in certain embodiments, the amino acid comprising the primary amine may be 5-aminopentanoic acid. (Exemplary linker containing 5-aminopentanoic acid is shown in FIG. 24) In certain embodiments, Y may have the structure —(CH₂)ₙ—, wherein n is 5. That is, in certain embodiments, the amino acid comprising the primary amine may be 6-aminohexanoic acid.

In certain embodiments, Y may have the structure —(CH₂)ₙ—, wherein n is 6. That is, in certain embodiments, the amino acid comprising the primary amine may be 7-aminoheptanoic acid.

In certain embodiments, Y may have the structure —(CH₂)ₙ—, wherein n is 7. That is, in certain embodiments, the amino acid comprising the primary amine may be 8-aminooctanoic acid.

In certain embodiments, Y may have the structure —(CH₂)ₙ—, wherein n is 8. That is, in certain embodiments, the amino acid comprising the primary amine may be 9-aminononanoic acid.

In certain embodiments, Y may have the structure —(CH₂)ₙ—, wherein n is 9. That is, in certain embodiments, the amino acid comprising the primary amine may be 10-aminodecanoic acid.

In certain embodiments, Y may have the structure —(CH₂)ₙ—, wherein n is 10. That is, in certain embodiments, the amino acid comprising the primary amine may be 11-aminoundecanoic acid.

In certain embodiments, the amino acid comprising the primary amine may have the structure NH₂—(CH₂)ₙ—X—(CH₂)ₙ—COOH, wherein X is a substituted or unsubstituted alkyl or heteroalkyl chain and wherein n is an integer from 0-20, from 0-10 or from 0-6.

That is, in certain embodiments, the amino acid comprising the primary amine may have the structure NH₂—(CH₂)ₙ—X—COOH, wherein X is a substituted or unsubstituted alkyl or heteroalkyl chain and wherein n is an integer from 1-20, from 1-10 or from 1-6.

In certain embodiments, the amino acid comprising the primary amine may have the structure NH₂—X—(CH₂)ₙ—COOH, wherein X is a substituted or unsubstituted alkyl or heteroalkyl chain and wherein n is an integer from 1-20, from 1-10 or from 1-6.

In a preferred embodiment, the amino acid comprising the primary amine comprises at least one methylene group (CH₂). More preferably, the at least one methylene group is directly coupled to the primary amine. That is, the amino acid comprising the primary amine preferably comprises the structure NH₂—CH₂—.

In a preferred embodiment, in the peptide linker according to the invention, the primary amine comprised in the amino acid residue is
a) a primary amine in a side chain of a lysine, a lysine derivative or a lysine mimetic; or
b) a primary amine comprised in an N-terminal amino acid residue having the structure NH2—(CH₂)ₙ—COOH, wherein n is an integer ranging from 1 to 10.

In certain embodiments, a payload is directly or indirectly attached to the N-terminal end of the peptide linker. In such embodiments, it is preferred that the primary amine comprised in the amino acid residue is a primary amine in a side chain of a lysine, a lysine derivative or a lysine mimetic; more preferably a primary amine in a side chain of a lysine residue.

In a particular embodiment, in the peptide linker according to the invention, the linker comprises not more than 25, 20, 15, 14, 13, 12, 11, 10, 9, 8, 7, 6, 5, 4 amino acid residues.

The peptide linker in accordance with the present invention preferably comprises at least two amino acid residues and not more than 25 amino acid residues. In some embodiments, all amino acid residues comprised in the peptide linker according to the invention form a single peptide. However, it is to be understood that the peptide linker may comprise two or more peptide moieties. For example, in certain embodiments, the peptide linker may comprise two peptide moieties, wherein the two peptide moieties are connected to each other covalently, but not by a peptide bond. Examples of such peptide linkers will be given further below.

In a particular embodiment, in the peptide linker in accordance with the present invention, the net charge of the linker is neutral or positive.

The net charge of a peptide is usually calculated at neutral pH (7.0). In the simplest approach, the net charge is determined by adding the number of positively charged amino acid residues (Arg, Lys and His) and the number of negatively charged ones (Asp and Glu) and calculate the difference of the two groups. In cases where the linker comprises non-canonical amino acids or amino acid derivatives comprising a charged functional group, the skilled person is capable of calculating the net charge at neutral pH accordingly.

In certain embodiments, the payloads may also contribute to the net charge of the linker. However, the skilled person is aware of methods to calculate the net charge of the entire linker, including any payloads, preferably at neutral pH (7.0).

In certain embodiments, the net charge of a peptide linker is calculated solely based on the amino acid residues comprised in the linker, including amino acid mimetics and amino acid derivatives. Thus, in a particular embodiment, in the peptide linker in accordance with the present invention, the net charge of the amino acid residues comprised in the peptide linker is neutral or positive.

In a particular embodiment, in the peptide linker in accordance with the present invention, the linker comprises no negatively-charged amino acid residues.

That is, the linker may be free of negatively charged amino acid residues, including negatively-charged amino acid mimetics and amino acid derivatives. A negatively charged amino acid residue is an amino acid, amino acid mimetic or amino acid derivative which carries a negative charge at neutral pH (7.0). Negatively charged canonical amino acids are glutamic acid and aspartic acid. However, negatively charged non-canonical amino acids, amino acid mimetics and amino acid derivatives are known in the art.

It has to be noted that the peptide linker in accordance with the present invention may comprise one or more glutamate or aspartate residue. However, in such embodiments, it is preferred that the carboxyl group comprised in the aspartate or glutamate side chain is coupled to a payload.

In a particular embodiment, in the peptide linker in accordance with the present invention, the linker comprises at least one positively-charged amino acid residue.

In certain embodiments, the peptide linker comprises a positively charged lysine residue, which provides the primary amine for the transglutaminase-mediated conjugation to an antibody. However, it is preferred herein that the peptide linker comprises at least one additional positively charged amino acid. The additional positively charged amino acid may be a canonical amino acid residue, such as arginine or histidine. However, the additional positively charged amino acid may also be a non-canonical amino acid.

In a particular embodiment, in the peptide linker in accordance with the present invention, the linker comprises at least one arginine residue.

It has been demonstrated herein that linkers comprising an arginine residue can be conjugated to glycosylated antibodies with high efficiency. Thus, it is preferred herein that the peptide linker in accordance with the present invention comprises at least one arginine residue. It is to be noted that the arginine residue may also be replaced by an arginine mimetic or arginine derivative.

The arginine residue may be located at any position of the peptide linker. In certain embodiments, the arginine residue is adjacent to the amino acid residue comprising the primary amine. In certain embodiments, the arginine residue is coupled to the N-terminus of the amino acid comprising the primary amine, i.e., a lysine residue, a lysine mimetic or a lysine derivative (e.g., RK motif). In certain embodiments, the arginine residue is coupled to the C-terminus of the amino acid comprising the primary amine, i.e., a lysine residue, a lysine mimetic or a lysine derivative (e.g., KR motif). In certain embodiments, the arginine residue is coupled to the amino acid comprising the primary amine, i.e., a lysine residue, a lysine mimetic or a lysine derivative, via another amino acid residue, preferably an alanine residue (KAR or RAK motif). In certain embodiments, the peptide linker comprises an arginine and a histidine residue.

In a particular embodiment, in the in the peptide linker in accordance with the present invention, the linker comprises at least one histidine residue.

It has been demonstrated in WO 2023/161291, which is incorporated herein in its entirety, that linkers comprising a histidine residue can be conjugated to glycosylated antibodies with high efficiency. Thus, it is preferred herein that the peptide linker according to the invention comprises at least one histidine residue. It is to be noted that the histidine residue may also be replaced by a histidine mimetic or histidine derivative.

The histidine residue may be located at any position of the peptide linker. In certain embodiments, the histidine residue is adjacent to the amino acid residue comprising the primary amine. In certain embodiments, the histidine residue is coupled to the N-terminus of the amino acid comprising the primary amine, i.e., a lysine residue, a lysine mimetic or a lysine derivative (e.g., HK motif). In certain embodiments, the histidine residue is coupled to the C-terminus of the amino acid comprising the primary amine, i.e., a lysine residue, a lysine mimetic or a lysine derivative (e.g., KH motif). In certain embodiments, the histidine residue is coupled to the amino acid comprising the primary amine, i.e., a lysine residue, a lysine mimetic or a lysine derivative, via another amino acid residue, preferably an alanine residue (KAH or HAK motif). In certain embodiments, the peptide linker comprises a histidine and an arginine residue.

In a particular embodiment, in the peptide linker in accordance with the present invention, the linker comprises the sequence motif RK.

Peptide linkers comprising the sequence motif RK (argynyl-lysyl) can be conjugated to glycosylated antibodies with exceptionally high efficiency, even if the linker comprises two or more payloads. It is to be understood that the lysine residue comprised in the RK motif contains the primary amine via which the peptide linker is conjugated to a glutamine residue comprised in the antibody. That is, the lysine residue comprised in the RK motif is preferably the amino acid comprising the primary amine.

It is preferred herein that the motif RK consists of the amino acids arginine and lysine. However, it is to be understood that the arginine and/or the lysine residue may be substituted with an arginine mimetic/derivative and/or a lysine mimetic/derivative.

That is, in certain embodiments, the motif RK may comprise an arginine mimetic. The term “arginine mimetic”, as used herein, refers to a compound that has a structure that is different from arginine, but that has similar characteristics as arginine and may thus be used to replace arginine in a peptide or protein without significantly altering the function and/or structure of said peptide or protein. An arginine mimetic may differ from arginine in length or composition of the aliphatic chain that connects the guanidino group and the α-carbon atom. Alternatively, or in addition, arginine mimetics may differ from arginine in the guanidino group itself. That is, the arginine mimetic may comprise a functional group with similar physicochemical properties as the guanidino group. In certain embodiments, the arginine mimetic may be homoarginine, 2-amino-3-guanidino-propionic acid, β-ureidoalanine or citrulline.

In certain embodiments, the motif RK may comprise an arginine derivative. The term “arginine derivative”, as used herein, refers to an arginine or arginine mimetic, wherein one or more functional groups comprised in the arginine or arginine mimetic is (are) modified or substituted. An arginine derivative may be arginine or an arginine mimetic, wherein the guanidino group is substituted or modified. In certain embodiments, the arginine derivative may be ω-methylarginine. In embodiments, where the residue R is located in the N-terminal position of the linker, R may be an arginine derivative wherein the α-amino group is modified or substituted. In certain embodiments the α-amino group of the arginine or arginine mimetic may be acetylated.

In certain embodiments, the motif RK may comprise a lysine mimetic or lysine derivative as defined elsewhere herein.

In certain embodiments, the motif RK may comprise a lysine mimetic/derivative and an arginine mimetic/derivative.

In certain embodiments, the lysine residue, or the lysine mimetic or lysine derivative, may be separated from the arginine residue, or the arginine mimetic or arginine derivative, by one amino acid residue. That is, the peptide linker of the invention may comprise the sequence motif RXK or KXR, wherein X may be any amino acid. In a preferred embodiment, the lysine residue, or the lysine mimetic or lysine derivative, may be separated from the arginine residue, or the arginine mimetic or arginine derivative, by an alanine residue. That is, the peptide linker of the invention may comprise the sequence motif RAK or KAR. It has been demonstrated in WO 2023/161291, which is incorporated herein in its entirety, that a linker comprising the sequence motif KAR can be conjugated to glycosylated antibodies with exceptionally high conjugation efficiency.

In a particular embodiment, in the peptide linker in accordance with the present invention, the linker comprises the sequence motif HK.

Peptide linkers comprising the sequence motif HK (histidyl-lysyl) can be conjugated to glycosylated antibodies with very high efficiency, even if the linker comprises two or more payloads. It is to be understood that the lysine residue comprised in the HK motif contains the primary amine via which the peptide linker is conjugated to a glutamine residue comprised in the antibody. That is, the lysine residue comprised in the HK motif is preferably the amino acid comprising the primary amine.

It is preferred herein that the motif HK consists of the amino acids histidine and lysine. However, it is to be understood that the histidine and/or the lysine residue may be substituted with a histidine mimetic/derivative and/or a lysine mimetic/derivative.

That is, in certain embodiments, the motif HK may comprise a histidine mimetic. The term “histidine mimetic”, as used herein, refers to a compound that has a structure that is different from histidine, but that has similar characteristics as histidine and may thus be used to replace histidine in a peptide or protein without significantly altering the function and/or structure of said peptide or protein. A histidine mimetic may differ from histidine in length or composition of the aliphatic chain that connects the imidazole group and the α-carbon atom. Alternatively, or in addition, histidine mimetics may differ from histidine in the imidazole group itself. That is, the histidine mimetic may comprise a functional group with similar physicochemical properties as the imidazole group. In certain embodiments, the histidine mimetic may be homohistidine.

In certain embodiments, the motif HK may comprise a histidine derivative. The term “histidine derivative”, as used herein, refers to a histidine or histidine mimetic, wherein one or more functional groups comprised in the histidine or histidine mimetic is (are) modified or substituted. A histidine derivative may be histidine or a histidine mimetic, wherein the imidazole group is substituted or modified. In embodiments, where the residue H is located in the N-terminal position of the linker, H may be a histidine derivative wherein the α-amino group is modified or substituted. In certain embodiments the α-amino group of the histidine or histidine mimetic may be acetylated.

In certain embodiments, the motif HK may comprise a lysine mimetic or lysine derivative as defined elsewhere herein.

In certain embodiments, the motif HK may comprise a lysine mimetic/derivative and a histidine mimetic/derivative.

In certain embodiments, the lysine residue, or the lysine mimetic or lysine derivative, may be separated from the arginine residue, or the arginine mimetic or arginine derivative, by one amino acid residue. That is, the peptide linker of the invention may comprise the sequence motif HXK or KXH, wherein X may be any amino acid. In a preferred embodiment, the lysine residue, or the lysine mimetic or lysine derivative, may be separated from the arginine residue, or the arginine mimetic or arginine derivative, by an alanine residue. That is, the peptide linker of the invention may comprise the sequence motif HAK or KAH.

In a particular embodiment, in the peptide linker in accordance with the present invention, the linker comprises any one of the amino acid sequences set forth in SEQ ID NO:1-29 or 82-93.

That is, the peptide linker may comprise any one of the amino acid sequences set forth in SEQ ID NOs: 1-29 or 82-93.

That is in certain embodiments, the peptide linker may comprise the peptide sequence RKAA (SEQ ID NO:1). Several linkers comprising the sequence RKAA have been shown herein (see FIGS. 4, 6 and 7). Preferably, one or more payloads are attached to the N- and/or C-terminus of the peptide RKAA.

In certain embodiments, the peptide linker may comprise the peptide sequence RK. A linker comprising the sequence RK is shown herein (see FIG. 8). Preferably, one or more payloads are attached to the N- and/or C-terminus of the peptide RK.

In certain embodiments, the peptide linker may comprise the peptide sequence ARK (SEQ ID NO:2). Preferably, one or more payloads are attached to the N- and/or C-terminus of the peptide ARK.

In certain embodiments, the peptide linker may comprise the peptide sequence RKARA (SEQ ID NO:3). Preferably, one or more payloads are attached to the N- and/or C-terminus of the peptide RKARA.

In certain embodiments, the peptide linker may comprise the peptide sequence RKAAAA (SEQ ID NO:4). Preferably, one or more payloads are attached to the N- and/or C-terminus of the peptide RKAAAA.

In certain embodiments, the peptide linker may comprise the peptide sequence RKAAAAAA (SEQ ID NO:5). Preferably, one or more payloads are attached to the N- and/or C-terminus of the peptide RKAAAAAA.

In certain embodiments, the peptide linker may comprise the peptide sequence RKAASGSG (SEQ ID NO:6). Preferably, one or more payloads are attached to the N- and/or C-terminus of the peptide RKAASGSG.

In certain embodiments, the peptide linker may comprise the peptide sequence RKHA (SEQ ID NO:7). Preferably, one or more payloads are attached to the N- and/or C-terminus of the peptide RKHA.

In certain embodiments, the peptide linker may comprise the peptide sequence RKHAAA (SEQ ID NO:8). Preferably, one or more payloads are attached to the N- and/or C-terminus of the peptide RKHAAA.

In certain embodiments, the peptide linker may comprise the peptide sequence GGR (SEQ ID NO:9). Preferably, one or more payloads are attached to the N- and/or C-terminus of the peptide GGR.

In certain embodiments, the peptide linker may comprise the peptide sequence GGRG (SEQ ID NO:10). Preferably, one or more payloads are attached to the N- and/or C-terminus of the peptide GGRG.

In certain embodiments, the peptide linker may comprise the peptide sequence EARKAA (SEQ ID NO:11). Preferably, one or more payloads are attached to the N- and/or C-terminus of the peptide EARKAA. In addition, it is preferred that one or more payloads are attached to the side chain of the glutamate residue. It is to be understood that when an amine comprising payload is attached to the side chain of the glutamate residue, the peptide sequence of the linker may also be viewed as QARKAA (SEQ ID NO:84).

In certain embodiments, the peptide linker may comprise the peptide sequence RKAEA (SEQ ID NO:12). Preferably, one or more payloads are attached to the N- and/or C-terminus of the peptide RKAEA. In addition, it is preferred that one or more payloads are attached to the side chain of the glutamate residue. It is to be understood that when an amine comprising payload is attached to the side chain of the glutamate residue, the peptide sequence of the linker may also be viewed as RKAQA (SEQ ID NO:85).

In certain embodiments, the peptide linker may comprise the peptide sequence HKA (SEQ ID NO:13). Preferably, one or more payloads are attached to the N- and/or C-terminus of the peptide HKA.

In certain embodiments, the peptide linker may comprise the peptide sequence RhKAA (SEQ ID NO:14), wherein hK is homolysine. Preferably, one or more payloads are attached to the N- and/or C-terminus of the peptide RhKAA.

In certain embodiments, the peptide linker may comprise the peptide sequence XGRG (SEQ ID NO:15), wherein X has the structure NH2—(CH₂)ₙ—COOH, wherein n is an integer from 1-20, preferably from 1-10. A linker comprising the sequence XGRG is exemplified in FIG. 24. Preferably, one or more payloads are attached to the C-terminus of the peptide XGRG.

In certain embodiments, the peptide linker may comprise the peptide sequence RKVCit (SEQ ID NO:16), wherein Cit is citrulline. Preferably, one or more payloads are attached to the N- and/or C-terminus of the peptide RKVCit.

In certain embodiments, the peptide linker may comprise the peptide sequence RKAR (SEQ ID NO:17). Preferably, one or more payloads are attached to the N- and/or C-terminus of the peptide RKAR.

In certain embodiments, the peptide linker may comprise the peptide sequence RKVA (SEQ ID NO:18). Preferably, one or more payloads are attached to the N- and/or C-terminus of the peptide RKVA.

In certain embodiments, the peptide linker may comprise the peptide sequence KAR (SEQ ID NO:19). Preferably, one or more payloads are attached to the N- and/or C-terminus of the peptide KAR.

In certain embodiments, the peptide linker may comprise the peptide sequence RKEAA (SEQ ID NO:20). Preferably, one or more payloads are attached to the N- and/or C-terminus of the peptide RKEAA. In addition, it is preferred that one or more payloads are attached to the side chain of the glutamate residue. It is to be understood that when an amine comprising payload is attached to the side chain of the glutamate residue, the peptide sequence of the linker may also be viewed as RKQAA (SEQ ID NO:86).

In certain embodiments, the peptide linker may comprise the peptide sequence RKDA (SEQ ID NO:82). Preferably, one or more payloads are attached to the N- and/or C-terminus of the peptide RKDA. In addition, it is preferred that one or more payloads are attached to the side chain of the aspartate residue. It is to be understood that when an amine comprising payload is attached to the side chain of the aspartate residue, the peptide sequence of the linker may also be viewed as RKNA (SEQ ID NO:83).

In certain embodiments, the peptide linker may comprise the peptide sequence ERKAA (SEQ ID NO:21). Preferably, one or more payloads are attached to the N- and/or C-terminus of the peptide ERKAA. In addition, it is preferred that one or more payloads are attached to the side chain of the glutamate residue. It is to be understood that when an amine comprising payload is attached to the side chain of the glutamate residue, the peptide sequence of the linker may also be viewed as QRKAA (SEQ ID NO:87).

In certain embodiments, the peptide linker may comprise the peptide sequence RKAH (SEQ ID NO:22). Preferably, one or more payloads are attached to the N- and/or C-terminus of the peptide RKAH.

In certain embodiments, the peptide linker may comprise the peptide sequence RKAN (SEQ ID NO:23). Preferably, one or more payloads are attached to the N- and/or C-terminus of the peptide RKAN.

In certain embodiments, the peptide linker may comprise the peptide sequence RKGGFG (SEQ ID NO:24). Preferably, one or more payloads are attached to the N- and/or C-terminus of the peptide RKGGFG.

In certain embodiments, the peptide linker may comprise the peptide sequence RKGP (SEQ ID NO:25). Preferably, one or more payloads are attached to the N- and/or C-terminus of the peptide RKGP.

In certain embodiments, the peptide linker may comprise the peptide sequence KRKAA (SEQ ID NO:26). Preferably, one or more payloads are attached to the N- and/or C-terminus of the peptide KRKAA. In addition, it is preferred that one or more payloads are attached to the side chain of one of the lysine residues, preferably the N-terminal lysine residue.

In certain embodiments, the peptide linker may comprise the peptide sequence SRKAA (SEQ ID NO:27). Preferably, one or more payloads are attached to the N- and/or C-terminus of the peptide SRKAA. In addition, it is preferred that one or more payloads are attached to the side chain of the serine residue.

In certain embodiments, the peptide linker may comprise the peptide sequence DDRKAA (SEQ ID NO:28). Preferably, one or more payloads are attached to the N- and/or C-terminus of the peptide DDRKAA. In addition, it is preferred that one or more payloads are attached to the side chains of the aspartate residues. It is to be understood that when an amine comprising payload is attached to a side chain of an aspartate residue, the peptide sequence of the linker may also be viewed as DNRKAA (SEQ ID NO:88), NDRKAA (SEQ ID NO:89) or NNRKAA (SEQ ID NO:90).

In certain embodiments, the peptide linker may comprise the peptide sequence EERKVaICit (SEQ ID NO:29). Preferably, one or more payloads are attached to the N- and/or C-terminus of the peptide EERKVaICit. In addition, it is preferred that one or more payloads are attached to the side chains of the glutamate residues. It is to be understood that when an amine comprising payload is attached to a side chain of a glutamate residue, the peptide sequence of the linker may also be viewed as EQRKVaICit (SEQ ID NO:91), QERKVaICit (SEQ ID NO:92) or QQRKVaICit (SEQ ID NO:93).

In certain embodiments, the peptide linker is any one of the linkers shown in FIGS. 4, 6, 7 and 8.

In a particular embodiment, the peptide linker in accordance with the present invention comprises between 2 and 4 payloads.

In a particular embodiment, the peptide linker in accordance with the present invention comprises or contains 2 payloads, i.e., a as a first payload a single topoisomerase I inhibitor which is cell-permeable; and as a second payload a single topoisomerase I inhibitor which is not cell-permeable.

In a particular embodiment, the peptide linker in accordance with the present invention comprises or contains 3 payloads, i.e., as a first payload two topoisomerase I inhibitors which are cell-permeable; and as a second payload a single topoisomerase I inhibitor which is not cell-permeable.

In a particular embodiment, the peptide linker in accordance with the present invention comprises or contains 3 payloads, i.e., as a first payload a single topoisomerase I inhibitor which is cell-permeable; and as a second payload two topoisomerase I inhibitors which are not cell-permeable.

In a particular embodiment, the peptide linker in accordance with the present invention comprises or contains 4 payloads, i.e., as a first payload a single topoisomerase I inhibitor which is cell-permeable; and as a second payload three topoisomerase I inhibitors which are not cell-permeable.

In a particular embodiment, the peptide linker in accordance with the present invention comprises or contains 4 payloads, i.e., as a first payload two topoisomerase I inhibitors which are cell-permeable; and as a second payload two topoisomerase I inhibitors which are not cell-permeable.

In a particular embodiment, the peptide linker in accordance with the present invention comprises or contains 4 payloads, i.e., as a first payload three topoisomerase I inhibitors which are cell-permeable; and as a second payload a single topoisomerase I inhibitor which is not cell-permeable.

In a preferred particular embodiment, the peptide linker in accordance with the present invention comprises or contains 2 payloads, i.e., a as a first payload a single camptothecin cytotoxic molecule which is cell-permeable; and as a second payload a single camptothecin cytotoxic molecule which is not cell-permeable.

In a preferred particular embodiment, the peptide linker in accordance with the present invention comprises or contains 3 payloads, i.e., as a first payload two camptothecin cytotoxic molecules which are cell-permeable; and as a second payload a single camptothecin cytotoxic molecule which is not cell-permeable.

In a preferred particular embodiment, the peptide linker in accordance with the present invention comprises or contains 3 payloads, i.e., as a first payload a single camptothecin cytotoxic molecule which is cell-permeable; and as a second payload two camptothecin cytotoxic molecules which are not cell-permeable.

In a preferred particular embodiment, the peptide linker in accordance with the present invention comprises or contains 4 payloads, i.e., as a first payload a single camptothecin cytotoxic molecule which is cell-permeable; and as a second payload three camptothecin cytotoxic molecules which are not cell-permeable.

In a preferred particular embodiment, the peptide linker in accordance with the present invention comprises or contains 4 payloads, i.e., as a first payload two camptothecin cytotoxic molecules which are cell-permeable; and as a second payload two camptothecin cytotoxic molecules which are not cell-permeable.

In a preferred particular embodiment, the peptide linker in accordance with the present invention comprises or contains 4 payloads, i.e., as a first payload three camptothecin cytotoxic molecules which are cell-permeable; and as a second payload a single camptothecin cytotoxic molecule which is not cell-permeable.

The peptide linker in accordance of the present invention may be used for the generation of antibody-payload conjugates having a payload-to-antibody ratio of 4 or higher by means of a microbial transglutaminase. Native glycosylated antibodies have a single conjugation site at glutamine residue 295 (Q295) of the heavy chain. Since antibodies comprise two heavy chains, conjugating a linker with two payloads to each of the glutamine residues results in an antibody-payload conjugate comprising 4 payloads. Analogously, conjugating a linker with three or four payloads to each of the glutamine residues results in an antibody-payload conjugate comprising 6 or 8 payloads, respectively. Thus, in certain embodiments, the peptide linker according to the invention comprises 2, 3 or 4 payloads.

The inventors identified different ways to couple two or more payloads to a peptide linker. In certain embodiments, two payloads may be coupled to the C-terminal end of a peptide linker. In other embodiments two payloads may be coupled to the N-terminal end of a peptide linker. In yet another embodiment, one or two payloads may be coupled each to the N-terminal end of a peptide linker and to the C-terminal end of a peptide linker.

In certain embodiments, the peptide linker is the linker shown in FIG. 8.

In embodiments where no payload is attached to the N- or C-terminus of a peptide linker, it is preferred that the respective terminus is modified. That is, the N-terminus of a peptide linker is preferably acetylated and the C-terminus of a peptide linker is preferably amidated.

In addition to coupling payloads to the terminal ends of a peptide linker, one or more payloads may also be coupled to amino acid side chains. The skilled person is aware of amino acid residues having functional groups in their amino acid side chains that allow for coupling of a payload. Amino acids having functional groups in their side chains include, but are not limited to, those described by deGruiter et al in Biochemistry 2017, 56, 30, 3863-3873. Additionally, payloads may also be coupled to the side chain of non-canonical amino acids, including but not limited to pAcF, CpK, pAMF, SCpHK, AzK, Sec.

That is, in a particular embodiment, in the peptide linker in accordance with the present invention, the at least one payload is attached to a side chain of a glutamate, aspartate, tryptophan, cysteine, lysine, tyrosine, serine or threonine residue comprised in the peptide linker.

In a particular embodiment, one or two payloads may be attached to the carboxylic acid of a glutamate or aspartate side chain.

In a particular embodiment, one or two payloads may be attached to the amine of a lysine side chain.

In a particular embodiment, one or two payloads may be attached to the thiol of a cysteine side chain.

In a particular embodiment, one or two payloads may be attached to the hydroxyl of a serine, threonine, or tyrosine side chain.

The payloads may be directly coupled to the peptide linker. For example, an amine-comprising payload may be coupled to the C-terminal end of a peptide linker via an isopeptide bond. Similarly, a carboxyl-comprising payload may be coupled to the N-terminal end of a peptide linker via an isopeptide bond or a thiol-comprising payload may be coupled to the side chain of a cysteine residue comprised in the peptide linker.

However, it is preferred herein that the payloads are coupled to the peptide linker via a chemical linker. In particular when two payloads are to be attached either to the N-terminal end or the C-terminal end of the peptide linker, the use of a chemical linker between the two payloads and the N- or C-terminal end is preferred.

Accordingly, in a particular embodiment, at least one of the two or more payloads is attached to the peptide linker via a chemical linker.

Within the present invention, it is preferred that at least one of the two or more payloads is coupled to the peptide linker via a chemical linker. However, even more preferably, all payloads are coupled to the peptide linker via a chemical linker.

A chemical linker can have various purposes. In certain embodiments, the chemical linker merely functions as an “adapter” to couple one payload to a peptide linker. For example, a chemical linker comprising an amine group may be used for coupling a payload to the C-terminal end of a peptide linker via an amide bond. In such embodiments, it is preferred that the chemical linker comprises one or more functional groups other than the amine to allow coupling of the payloads to the chemical linker via these additional functional groups.

In certain embodiments, the chemical linker functions as an “amplifier moiety” to couple several payloads to a peptide linker. For example, a chemical linker comprising a disubstituted amine may be used as a dendron to attach two payloads. The disubstituted amine can serve as a branching point, allowing for the attachment of multiple payload molecules, thereby increasing the drug-to-antibody ratio (DAR). The chemical linker comprising the disubstituted amine may have the following structure:
[payload]-NH-[payload]
wherein each [payload] is directly or indirectly linked to the nitrogen (N).

The disubstituted amine may be linked to a carboxyl group comprised in the linker via an amide bond. Preferably, the disubstitited amine is linked to the C-terminal end of the peptide linker or to the N-terminal end of the peptide linker via a dicarboxylic acid as described elsewhere herein. An example of an amplifier comprising a disubstituted amine is the N-(2-Carboxyethyl)-Alanine (CEA) moiety.

An example of such an amplifier is illustrated in FIG. 8.

Another example of an amplifier is the 2,6-bis-(hydroxymethyl)-p-cresol moiety. For example, a chemical linker comprising a disubstituted carboxylic acid may be used as a dendron to attach two payloads.

Additional strategies for coupling two or more payloads to a peptide linker are disclosed in WO 2023/161291, which is incorporated herein in its entirety.

Similarly, chemical linkers comprising a carboxyl group may be used for coupling one or more payload to the N-terminal end of a peptide linker via an amide bond. For example, a dicarboxylic acid molecule may be used for coupling an amine-comprising payload to the N-terminal end of a peptide.

Further, chemical linkers comprising a compatible functional group may be used for coupling a payload to an amino acid side chain comprising in a peptide linker.

In any of the embodiments disclosed above, the skilled person is capable of identifying a chemical linker that is suitable for coupling a payload to a peptide linker, whether the chemical serves as an “adapter” or an “amplifier moiety”. That is, the skilled person is able to identify a linker having the functional groups that are required for coupling a payload of interest to a functional group comprised in the peptide linker.

However, the chemical linker may not only function as an adapter between the payload(s) and the peptide linker, but also fulfill other functions.

That is, in certain embodiments, the chemical linker is an enzymatically and/or chemically cleavable linker.

The cleavable linker may be any enzymatically and/or chemically cleavable linker known in the art, including, but not limited to, those described by Bargh et al (Chem. Soc. Rev., 2019, 48, 4361), which is fully incorporated herein by reference.

Cleavable linkers have the advantage that the release of the payloads from the antibody can be controlled and/or facilitated. For example, one or more payloads may be coupled to the peptide linker via an enzymatically and/or chemically cleavable chemical linker.

In certain embodiments, the chemical linker is cleavable in vivo. Cleavable linkers may include chemically or enzymatically unstable or degradable linkages. Cleavable linkers generally rely on biological processes to liberate the payload, such as reduction in the cytoplasm, exposure to acidic conditions in the lysosome, or cleavage by specific proteases or other enzymes within, or outside, the cell. Cleavable linkers generally incorporate one or more chemical bonds that are either chemically or enzymatically cleavable. In certain embodiments, a linker comprises a chemically labile group such as hydrazone and/or disulfide groups. Linkers comprising chemically labile groups exploit differential properties between the plasma and some cytoplasmic compartments. The intracellular conditions to facilitate payload release for hydrazone containing linkers are the acidic environment of endosomes and lysosomes, while the disulfide containing linkers are reduced in the cytosol, which contains high thiol concentrations, e.g., glutathione. In certain embodiments, the plasma stability of a linker comprising a chemically labile group may be increased by introducing steric hindrance using substituents near the chemically labile group.

Acid-labile groups, such as hydrazone or carbonate, remain intact during systemic circulation in the blood's neutral pH environment (pH 7.3-7.5) and undergo hydrolysis and release the payload once the ADC is internalized into mildly acidic endosomal (pH 5.0-6.5) and lysosomal (pH 4.5-5.0) compartments of the cell. This pH dependent release mechanism has been associated with nonspecific release of the payload. To increase the stability of the hydrazone group of the linker, the linker may be varied by chemical modification, e.g., substitution, allowing tuning to achieve more efficient release in the lysosome with a minimized loss in circulation. Hydrazone- or carbonate-containing linkers may contain additional cleavage sites, such as additional acid-labile cleavage sites and/or enzymatically labile cleavage sites.

Other acid-labile groups that may be included in chemical linkers include cis-aconityl-containing linkers. Cis-Aconityl chemistry uses a carboxylic acid juxtaposed to an amide bond to accelerate amide hydrolysis under acidic conditions.

Cleavable chemical linkers may also include a disulfide group. Disulfides are thermodynamically stable at physiological pH and are designed to release the payload upon internalization inside cells, wherein the cytosol provides a significantly more reducing environment compared to the extracellular environment. Scission of disulfide bonds generally requires the presence of a cytoplasmic thiol cofactor, such as (reduced) glutathione (GSH), such that disulfide-containing linkers are reasonably stable in circulation, selectively releasing the payload in the cytosol. The intracellular enzyme protein disulfide isomerase, or similar enzymes capable of cleaving disulfide bonds, may also contribute to the preferential cleavage of disulfide bonds inside cells. GSH is reported to be present in cells in the concentration range of 0.5-10 mM compared with a significantly lower concentration of GSH or cysteine, the most abundant low-molecular weight thiol, in circulation at approximately 5 μM. Tumor cells, where irregular blood flow leads to a hypoxic state, result in enhanced activity of reductive enzymes and therefore even higher glutathione concentrations. In certain embodiments, the in vivo stability of a disulfide-containing linker may be enhanced by chemical modification of the linker, e.g., use of steric hinderance adjacent to the disulfide bond.

Another type of cleavable linker that may be used is a chemical linker that is specifically cleaved by an enzyme. Such linkers are typically peptide-based or include peptidic regions that act as substrates for enzymes. Peptide based linkers tend to be more stable in plasma and extracellular milieu than chemically labile linkers. Peptide bonds generally have good serum stability, as lysosomal proteolytic enzymes have very low activity in blood due to endogenous inhibitors and the unfavorably high pH value of blood compared to lysosomes. Release of a payload from an antibody occurs specifically due to the action of lysosomal proteases, e.g., cathepsin, legumain, and plasmin. These lysosomal proteases may be present at elevated levels within certain tumor cells, but can also be found extracellularly, in the tumor microenvironment. Peptide-based linkers could also be cleaved by non-lysosomal extracellular proteases such as matrix metalloproteinases. Non-peptide-based linkers could also be specifically cleaved by glycosidases.

In exemplary embodiments, the cleavable peptide is selected from tetrapeptides such as Gly-Phe-Leu-Gly (SEQ ID NO:30), Ala-Leu-Ala-Leu (SEQ ID NO:31), Gly-Gly-Phe-Gly (SEQ ID NO:32) or dipeptides such as Ala-Ala, Ala-Arg, Val-Cit, Val-Ala, Met-(D)Lys, Asn-(D)Lys, Val-(D)Asp, Phe-Lys, IIe-Val, Asp-Val, His-Val, NorVal-(D)Asp, Ala-(D)Asp, Met-Lys, Asn-Lys, lie-Pro, Me3Lys-Pro, PhenylGly-(D)Lys, Met-(D)Lys, Asn-(D)Lys, Pro-(D)Lys, Met-(D)Lys, Asn-(D)Lys, Met-(D)Lys, Asn-(D)Lys. In certain embodiments, dipeptides are preferred over longer polypeptides due to hydrophobicity of the longer peptides. That is, linkers comprising an amino acid as set forth in SEQ ID NO:1-29 or 82-93 may further comprise any of the dipeptide or tetrapeptide motifs listed above. Preferably, the dipeptide or tetrapeptide motifs listed above are directly coupled to a payload or are coupled to a payload via a self-immolative spacer. However, it is to be understood that the peptide linker itself, such as any of the linkers comprising an amino acid as set forth in SEQ ID NO:1-29 or 82-93, may be subject to enzymatic cleavage by endogenous peptidases or proteases.

Enzymatically cleavable linkers may include a self-immolative spacer to spatially separate the payload from the site of enzymatic cleavage. The direct attachment of a payload to a peptide linker can result in proteolytic release of an amino acid adduct of the payload, thereby impairing its activity. The use of a self-immolative spacer allows for the elimination of the fully active, chemically unmodified payload upon amide or glycosidic bond hydrolysis.

In certain embodiments, the peptide linker in accordance with the present invention is a self-immolative linker which comprises:
a) a p-aminobenyzl alcohol moiety; or
b) a 2,4-bis(hydroxymethyl)aniline moiety; or
c) a p-aminobenzyl quaternary ammonium; or
d) an ethylenediamine-based moiety; or
e) an (aminomethyl)pyrrolidine-based moiety; or
f) an aminomethyl-based moiety.

One self-immolative spacer is the bifunctional para-aminobenzyl alcohol group, which is linked to the peptide through the amino group, forming an amide bond, while amine-containing drugs may be attached through carbamate functionalities to the benzylic hydroxyl group of the linker (PABC). The resulting prodrugs are activated upon protease-mediated cleavage, leading to a 1,6-elimination reaction releasing the unmodified drug, carbon dioxide, and remnants of the linker group. Heterocyclic variants of this self-immolative group have also been described. See for example, U.S. Pat. No. 7,989,434, incorporated herein by reference. The para-aminobenzyl alcohol moiety may also be used to link a phenol- or hydroxyl-containing payload through the formation of a carbonate. The para-aminobenzyl moiety may also be used to link a tertiary- or heteroaryl-amine-containing payload through the formation of a quaternary ammonium (PABQ). That is, in certain embodiments, in the peptide linker according to the invention, the quaternary ammonium cation comprised in the p-aminobenzyl quaternary ammonium originates from an amine comprised in the payload. Preferably, the amine comprised in the payload is a tertiary amine or a heteroaryl-amine.

Another self-immolative spacer is the 2,4-bis(hydroxymethyl)aniline group, which is linked to the peptide through the amino group, forming an amide bond, while amine-containing drugs may be attached through two carbamate functionalities via the two benzylic hydroxyl groups of the linker. The resulting prodrugs are activated upon protease-mediated cleavage, leading to payload release via successive 1,6- and 1,4-elimination processes.

For hydroxyl-containing drugs, suitable self-immolative spacers include, but are not limited to, ethylenediamine-based carbamate (EDA), (aminomethyl)pyrrolidine-based carbamate (AMP) (see FIG. 33), or the aminomethyl moeity (AM). The release mechanism of this latter utilizes the lability of the hemiaminal functionality, which readily undergoes 1,2-elimination to release the desired alcohol.

For thiol-containing drugs, suitable self-immolative spacers include, but are not limited to, the aminomethyl moeity (AM). The release mechanism of this latter utilizes the lability of the thiohemiaminal functionality, which readily undergoes 1,2-elimination to release the desired thiol.

In some embodiments, the enzymatically cleavable linker is a β-glucuronic acid-based linker. Facile release of the payload may be realized through cleavage of the β-glucuronide glycosidic bond by the lysosomal enzyme β-glucuronidase. This enzyme is present abundantly within lysosomes and is overexpressed in some tumor types, while the enzyme activity outside cells is low. β-Glucuronic acid-based linkers may be used to circumvent the tendency of an antibody-payload conjugate to undergo aggregation due to the hydrophilic nature of β-glucuronides.

As mentioned above, in a particular embodiment, the chemical linker is or comprises a self-immolative linker.

It is preferred herein that the payloads are attached to the peptide linker via a self-immolative linker to facilitate release of the unmodified drug. Even more preferably, the self-immolative linker is coupled to a peptide sequence that is efficiently cleaved by a protease or a peptidase.

The cleavable peptide may be defined as part of the peptide linker or as part of the chemical linker that connects the peptide linker with the payload(s).

The self-immolative linker may be any self-immolative linker known in the art. However, it is preferred that the self-immolative linker comprises a p-aminobenzyl alcohol moiety or a 2,4-bis(hydroxymethyl)aniline moiety.

That is, in a particular embodiment, the self-immolative linker comprises a p-aminobenzyl alcohol moiety or a 2,4-bis(hydroxymethyl)aniline moiety.

Self-immolative linkers comprising a p-aminobenyzl alcohol moiety may be used for coupling payloads to the C-terminus of a peptide. That is, the amino group of the p-aminobenzyl alcohol moiety may be coupled to the C-terminal carboxyl group of the peptide linker via an amide bond. Alternatively, or in addition, the amino group of the p-aminobenzyl alcohol moiety may be coupled to a carboxyl group in the side chain of an aspartate or glutamate residue in the peptide linker via an amide bond.

The payload may be coupled to the hydroxyl group of the p-aminobenzyl alcohol moiety via a carbamate. In certain embodiments, the C-terminal amino acid of the peptide linker to which the p-aminobenzyl alcohol moiety may be coupled may be comprised in a motif that is efficiently cleaved by a peptidase, such as, without limitation, the sequence motif valine-citrulline.

It is to be understood that the peptide linker in accordance with the present invention may comprise more than one p-aminobenzyl alcohol moiety. For example, a peptide linker according to the invention may comprise two peptide moieties, wherein the two peptide moieties are linked to each other via their N-terminal ends. In such embodiments, the peptide linker has two C-terminal ends and both C-terminal ends may be conjugated to a payload via a p-aminobenzyl alcohol moiety.

A p-aminobenzyl alcohol moiety may also be used for coupling a payload to an amino acid side chain. For example, a payload may be coupled to the carboxyl group in the side chain of a glutamate or aspartate residue via a p-aminobenzyl alcohol moiety. The p-aminobenzyl alcohol moiety may be coupled to the carboxyl group in the side chain of a glutamate or aspartate residue either directly or via one or more amino acid residues. In certain embodiment, the p-aminobenzyl alcohol moiety may be coupled to the carboxyl group in the side chain of a glutamate or aspartate residue via the valine-citrulline or alanine-alanine sequences.

In certain embodiments, an amine comprising payload may be coupled to a carboxyl group in the peptide linker by two or more aminobenzyl alcohol moieties.

Self-immolative linkers comprising a 2,4-bis(hydroxymethyl)aniline moiety may be used for coupling two payloads to a single functional group comprised in a peptide linker. That is, a 2,4-bis(hydroxymethyl)aniline moiety may be coupled to a carboxyl group comprised in a peptide linker via its amino group. Payloads may then be coupled to each of the hydroxyl groups via a carbamate.

By using linkers comprising 2,4-bis(hydroxymethyl)aniline moieties, peptide linkers comprising more than two payloads may be obtained. For example, a linker comprising four payloads may be obtained by coupling two payloads to the N-terminal end of a peptide linker via a 2,4-bis(hydroxymethyl)aniline moiety (indirectly via a second peptide moiety) and two more payloads to the C-terminal end of a peptide linker via another 2,4-bis(hydroxymethyl)aniline moiety. Similarly, peptide linkers comprising three payloads may be obtained by coupling two payloads to the peptide linker via a 2,4-bis(hydroxymethyl)aniline moiety and a third payload via a p-aminobenzyl alcohol moiety.

In a particular embodiment, the invention relates to the peptide linker according to the invention, wherein the hydroxyl group comprised in the p-aminobenzyl alcohol moiety forms a carbamate with a payload.

As mentioned above, a payload may be attached to the p-aminobenzyl alcohol moiety via a carbamate. That is, the payload preferably comprises a free amine group that is suitable to undergo formation of a carbamate. The skilled person is aware of methods to form a carbamate between a p-aminobenzyl alcohol moiety and an amine-comprising payload.

In a particular embodiment, in the peptide linker according to the invention, the hydroxyl group comprised in the p-aminobenzyl alcohol moiety forms a carbonate with a payload.

A payload may be attached to the p-aminobenzyl alcohol moiety via a carbonate. That is, the payload preferably comprises a free hydroxyl group that is suitable to undergo formation of a carbonate. The skilled person is aware of methods to form a carbonate between a p-aminobenzyl alcohol moiety and a hydroxyl-comprising payload.

In a particular embodiment, in the peptide linker according to the invention, each of the hydroxyl groups comprised in the 2,4-bis(hydroxymethyl)aniline moiety forms a carbamate with a payload.

That is, the 2,4-bis(hydroxymethyl)aniline moiety comprised in the peptide linker according to the invention may form two carbamates with two individual amine comprising payloads.

In a particular embodiment, in the peptide linker according to the invention, the p-aminobenzyl moiety forms a quaternary ammonium with a payload.

As mentioned above, a payload may be attached to the p-aminobenzyl via a quaternary ammonium. That is, the payload preferably comprises a tertiary- or a heteroaryl-amine that is suitable to undergo formation of a quaternary ammonium. The skilled person is aware of methods to form a quaternary ammonium between a p-aminobenzyl moiety and a tertiary- or a heteroaryl-amine-comprising payload.

In a particular embodiment, in the peptide linker according to the invention, the self-immolative linker comprises an ethylenediamine carbamate (EDA) moiety.

That is, payloads may be coupled to the peptide linker via an ethylenediamine carbamate (EDA) moiety. An EDA moiety may be coupled directly to the C-terminus of a peptide or to an aspartate or glutamate sidechain via an amide bond. EDA moieties preferably undergo carbamate formation with payloads comprising a hydroxyl group. An EDA moiety can also be used to connect an amplifier linked to two payloads.

In a particular embodiment, in the peptide linker according to the invention, the self-immolative linker comprises an (aminomethyl)pyrrolidine-based carbamate (AMP) moiety.

That is, payloads may be coupled to the peptide linker according to the invention via an (aminomethyl)pyrrolidine-based carbamate (AMP) moiety. An AMP moiety may be coupled directly to the C-terminus of a peptide or to an aspartate or glutamate sidechain via an amide bond. AMP moieties preferably undergo carbamate formation with payloads comprising a hydroxyl group.

In a particular embodiment, in the peptide linker according to the invention, the self-immolative linker comprises an aminomethyl (AM) moiety.

That is, payloads may be coupled to the peptide linker according to the invention via an aminomethyl (AM) moiety. An AM moiety may be coupled directly to the C-terminus of a peptide or to an aspartate or glutamate sidechain via an amide bond. AM moieties are preferably used to link payloads comprising a hydroxyl group, thereby forming a hemiaminal.

However, AM moieties can also be used to link payloads comprising a thiol group, thereby forming a thiohemiaminal.

In a particular embodiment, in the peptide linker according to the invention, at least one payload is attached to a side chain of a glutamate, aspartate, tryptophan, cysteine, lysine, tyrosine, serine, or threonine residue comprised in the peptide linker.

As mentioned above, one or more payloads may be coupled to an amino acid side chain comprised in the peptide linker. The skilled person is aware of chemical linkers that are suitable for coupling a payload to an amino acid side chain, i.e., the carboxyl group in the side chain of a glutamate or aspartate residue, the thiol group in the side chain of a cysteine residue, the amino group in the side chain of a lysine residue or the hydroxy group in the side chain of a tyrosine, serine, or threonine residue.

In a particular embodiment, the invention relates to the peptide linker according to the invention, wherein the peptide linker comprises two peptide moieties, and wherein the two peptide moieties are connected via their N-terminal amino acid residues with a dicarboxylic acid linker (HO₂C—R—CO₂H).

Certain linkers falling within the scope of the present invention comprise two peptide moieties, wherein the two peptide moieties are linked via their N-terminal amino acid residues. The N-terminal amino acids of the two peptide moieties may be linked via a dicarboxylic acid, wherein each carboxylic acid group comprised in the dicarboxylic acid forms an amide bond with an N-terminal amino group of a peptide moiety.

Any dicarboxylic acid may be used to link two peptide moieties via their N-terminal amino acid residues. In certain embodiments, the dicarboxylic acid may be an aliphatic dicarboxylic acid. That is, the dicarboxylic acid may be ethanedioic acid, propanedioic acid, butanedioic acid, pentanedioic acid, hexanedioic acid, heptanedioic acid, octanedioic acid, nonanedioic acid or decanedioic acid. In certain embodiments, two peptide moieties are linked via their N-terminal amino acids with a butanedioic acid molecule. In certain embodiments, two peptide moieties are linked via their N-terminal amino acids with a pentanedioic acid molecule. Aliphatic dicarboxylic acids may comprise substituted or unsubstituted alkyl or alkenyl chains.

In certain embodiments, the dicarboxylic acid may be an aromatic dicarboxylic acid. Aromatic dicarboxylic acids include, without limitation, phthalic acid, isophthalic acid, or terephthalic acid.

It is to be understood that linking two peptide moieties via their N-terminal amino acids results in a peptide construct comprising no free N-terminal amino group that may be suitable for conjugation to a glutamine residue comprised in an antibody. Thus, peptide linkers comprising two N-terminally linked peptide moieties preferably comprise a lysine residue, a lysine mimetic or a lysine derivative to enable conjugation of the peptide linker to a glutamine moiety comprised in an antibody.

In certain embodiments, the first peptide moiety comprised in the peptide linker may comprise any of the amino acid sequences set forth in SEQ ID NO:1-8, 11-14, 16-29 or 82-93.

The second peptide moiety may have any amino acid sequence. In certain embodiments, the second peptide moiety may have a length of 2-100, preferably 2-50, more preferably 2-25, even more preferably 2-10, most preferably 2-5 amino acid residues. In certain embodiments, the second peptide moiety may be a dipeptide or a tripeptide. However, it is to be noted that the second peptide moiety may also be a single amino acid or a longer peptide. To enable efficient release of the payload, the second peptide moiety preferably comprises a peptide sequence that is efficiently cleaved by a peptidase. In certain embodiments, the second peptide moiety may have the sequence Asn, Ala, Ala-Ala, Ala-Asn, Val-Ala, Val-Cit, Ala-Arg, Arg-Ala, Ala-Ala-Arg (SEQ ID NO:34), Ala-Arg-Ala (SEQ ID NO:35), Ala-Ala-Asn (SEQ ID NO:36).

That is, in certain embodiments, the peptide linker may have the structure:
[payload 1]-[peptide 1]-[dicarboxylic acid]-[peptide 2]-[payload 2]; wherein
[payload 1] and [payload 2] are payloads,
[peptide 1] is a first peptide moiety,
[peptide 2] is a second peptide moiety, and
[dicarboxylic acid] is a dicarboxylic acid;
wherein at least one of the peptide moieties 1 and/or 2 comprises a free amine,
wherein the N-terminal end of peptide 1 and the N-terminal end of peptide 2 are connected via the dicarboxylic acid,
wherein payload 1 is attached to the C-terminal end of peptide 1, preferably via a chemical linker, and
wherein payload 2 is attached to the C-terminal end of peptide 2, preferably via a chemical linker.

Preferably, the peptide moiety comprising a free amine group is a peptide moiety comprising a lysine residue, a lysine mimetic or a lysine derivative, as defined elsewhere herein or a peptide linker comprising any one of the amino acid sequences set forth in SEQ ID NO:1-8, 11-14, 16-29 or 82-93.

In certain embodiments, the peptide linker may comprise a first peptide moiety comprising a sequence set forth in SEQ ID NO:1-8, 11-14, 16-29 or 82-93 and a second moiety comprising the sequence Ala-Ala, wherein the first and second peptide moiety are linked via their N-terminal amino acids with a butanedioic acid molecule.

In certain embodiments, the peptide linker may comprise a first peptide moiety comprising the sequence RKAA and a second moiety comprising the sequence Ala-Ala, wherein the first and second peptide moiety are linked via their N-terminal amino acids with a butanedioic acid molecule.

Instead of coupling two peptide moieties via their N-terminal amino acid residues, a second peptide moiety may also be coupled to an amino acid side chain of a first peptide moiety. That is, the first peptide moiety comprised in the peptide linker may comprise any of the amino acid sequences set forth in SEQ ID NO:6, 11-12, 20-21, 23, 26-29 or 82-93. The second peptide moiety, that is the one positioned on the amino acid side chain, may have any amino acid sequence. In certain embodiments, the second peptide moiety may be a dipeptide or a tripeptide. However, it is to be noted that the second peptide moiety may also be a single amino acid or a longer peptide. To enable efficient release of the payload, the second peptide moiety preferably comprises a peptide sequence that is efficiently cleaved by a peptidase. In certain embodiments, the second peptide moiety may have the sequence Asn, Ala, Ala-Ala, Ala-Asn, Val-Ala, Val-Cit, Ala-Arg, Arg-Ala, Ala-Ala-Arg (SEQ ID NO:34), Ala-Arg-Ala (SEQ ID NO:35), Ala-Ala-Asn (SEQ ID NO:36).

The peptide linker according to the invention comprises two or more payloads. The peptide linkers comprising the two or more payloads are preferably obtained by chemical synthesis.

The skilled person is aware of methods for coupling a payload to an amino acid-based linker by chemical synthesis. For example, an amine-comprising payload (for e.g., auristatin analogs, exatecan), or a thiol-comprising payload (for e.g. maytansine analogs), or a hydroxyl-containing payload (for e.g. SN-38 analogs) may be attached to the C-terminus of an amino acid-based linker by chemical synthesis. However, the skilled person is aware of further reactions and reactive groups that may be utilized for coupling a payload to the N-terminus, C-terminus or the side chain of an amino acid or amino acid derivative by chemical synthesis. Typical reactions that may be used for coupling a payload to an amino acid-based linker by chemical synthesis include, without limitation: peptide coupling, activated ester coupling (NHS ester, PFP ester), click reaction (CuAAC, SPAAC), Michael addition (thiol maleimide conjugation).

The coupling of payloads to peptides has been extensively described in the prior art, for example by Costoplus et al. (Peptide-Cleavable Self-immolative Maytansinoid Antibody-Drug Conjugates Designed To Provide Improved Bystander Killing. ACS Med Chem Lett. 2019 Sep. 27; 10(10):1393-1399), Sonzini et al. (Improved Physical Stability of an Antibody-Drug Conjugate Using Host-Guest Chemistry. Bioconjug Chem. 2020 Jan. 15; 31(1):123-129), Bodero et al. (Synthesis and biological evaluation of RGD and isoDGR peptidomimetic-α-amanitin conjugates for tumor-targeting. Beilstein J. Org. Chem. 2018, 14, 407-415), Nunes et al. (Use of a next generation maleimide in combination with THIOMAB™ antibody technology delivers a highly stable, potent and near homogeneous THIOMAB™ antibody-drug conjugate (TDC). RSC Adv., 2017,7, 24828-24832), Doronina et al. (Enhanced activity of monomethylauristatin F through monoclonal antibody delivery: effects of linker technology on efficacy and toxicity. Bioconjug Chem. 2006 Jan-Feb;17(1):114-24), Nakada et al. (Novel antibody drug conjugates containing exatecan derivative-based cytotoxic payloads. Bioorg Med Chem Lett. 2016 Mar. 15; 26(6):1542-1545) and Dickgiesser et al. (Site-Specific Conjugation of Native Antibodies Using Engineered Microbial Transglutaminases. Bioconjug Chem. 2020 Mar 12. doi: 10.1021/acs.bioconjchem.0c00061).

It is to be understood that the payload may be coupled to the N-terminal and/or to the C-terminal end of a peptide-based or a peptide-comprising linker according to the invention. In certain embodiments, a payload may be coupled directly to the N-terminal amino group or the C-terminal carboxyl group of a peptide or an amino acid residue.

The skilled person is aware of reactive groups that are suitable for coupling a payload to an amino acid residue. For example, an amine-comprising payload may be coupled to the C-terminal carboxyl group of an amino acid residue via an amide bond. Alternatively, a payload comprising a thiol group or and hydroxyl group may be coupled to the C-terminal carboxyl group of an amino acid via a thioester or an ester bond, respectively. A payload comprising a carboxylic acid group may be coupled to the N-terminal amino group of an amino acid residue via an amide bond.

In certain embodiments, a payload may be coupled indirectly to the N- and/or C-terminal end of a peptide or amino acid residue comprised in the linker according to the invention. The skilled person is aware of linker molecules that may be used to couple a payload to the N-terminal amino group or the C-terminal carboxyl group of an amino acid residue comprised in the linker according to the invention.

In certain embodiments, a payload comprising a hydroxyl group may be coupled to the N-terminus of an amino acid residue via a linker molecule. For example, payloads comprising a hydroxyl group may be coupled to an N-terminal amino group via a carbamate linker.

In certain embodiments, a payload comprising a thiol group may be coupled to the N-terminus of an amino acid residue via a linker molecule. For example, payloads comprising a thiol group may be coupled to an N-terminal amino group via a thiocarbamate linker. Alternatively, payloads comprising a thiol group may be coupled to an N-terminal amino group via an alkyl linker molecule comprising a carboxyl group and a thiol group. In certain embodiments the alkyl linker molecule may be a 3-mercaptopropionic acid linker molecule, wherein the payload forms a di-sulfur bond with the thiol group comprised in the 3-mercaptopropionic acid linker molecule.

In certain embodiments, a payload comprising an amide group may be coupled to the N-terminus of an amino acid residue via a linker molecule. For example, payloads comprising an amine group may be coupled to an N-terminal amino group via a dicarboxylic acid linker molecule, wherein the each of the carboxylic acid groups comprised in the dicarboxylic acid linker forms an amide bond with the payload and the amino group of the N-terminal amino acid residue. Examples of dicarboxylic acids that may be used as linker molecules in the present invention are, without limitation, succinic acid or pimelic acid.

Alternative linker molecules for indirectly coupling payloads to the N-terminus of an amino acid residue comprised in the peptide linker according to the invention or linker molecules that are suitable for indirectly coupling payloads to the C-terminus of an amino acid residue comprised in the peptide linker according to the invention have been described in the art and are encompassed by the present invention.

In another particular embodiment, one or more payload is attached to the N-terminal end of an amine-comprising peptide linker and wherein one or more payload is attached to the C-terminal end of said amine-comprising peptide linker.

In certain embodiments, one or two payloads may be attached to the N-terminal end of an amine-comprising peptide linker and one or two payloads may be attached to the C-terminal end of said amine-comprising peptide linker. That is, the peptide linker may be a DAR4, DAR6 or DAR8 linker.

In certain embodiments, one payload may be attached to the N-terminal end of an amine-comprising peptide linker and one payload may be attached to the C-terminal end of said amine-comprising peptide linker. In such embodiments, the peptide linker may be a “linear” DAR4 linker.

The amine-comprising peptide linker maybe any one of the lysine-comprising peptide linkers disclosed herein, including peptide linkers comprising a lysine mimetic or a lysine derivative as defined herein.

In certain embodiments, the “linear” DAR4 linker may have the following structure (in N->C direction):

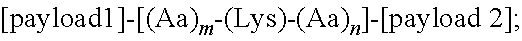

wherein
[payload 1] and [payload 2] are payloads,
Aa may be any amino acid residue;
m and n may be integers ranging from 0 to 10, preferably 0 to 6, more preferably 0 to 4; and
Lys is a lysine residue, a lysine mimetic or a lysine derivative,
wherein [payload 1] is directly or indirectly attached to an N-terminal end of an (Aa) or (Lys) residue, and
wherein [payload 2] is directly or indirectly attached to a C-terminal end of an (Aa) or (Lys) residue.

In certain embodiments, the “linear” DAR4 linker may have the following structure:

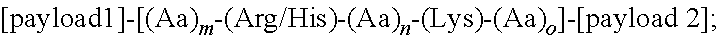

wherein
[payload 1] and [payload 2] are payloads,
Aa may be any amino acid residue;
m, n and o may be integers ranging from 0 to 10, preferably 0 to 6, more preferably 0 to 4; and
Arg may be an arginine residue, an arginine mimetic or an arginine derivative;
His may be a histidine residue, a histidine mimetic or a histidine derivative
Lys is a lysine residue, a lysine mimetic or a lysine derivative,
wherein [payload 1] is directly or indirectly attached to an N-terminal end of an (Aa) or (Arg/His) residue, and
wherein [payload 2] is directly or indirectly attached to a C-terminal end of an (Aa) or (Lys) residue.

In certain embodiments, the “linear” DAR4 linker may have the following structure:

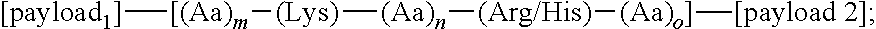

[payload 1] and [payload 2] are payloads,
Aa may be any amino acid residue;
m, n and o may be integers ranging from 0 to 10, preferably 0 to 6, more preferably 0 to 4; and
Arg may be an arginine residue, an arginine mimetic or an arginine derivative;
His may be a histidine residue, a histidine mimetic or a histidine derivative
Lys is a lysine residue, a lysine mimetic or a lysine derivative,
wherein [payload 1] is directly or indirectly attached to an N-terminal end of an (Aa) or (Lys) residue, and
wherein [payload 2] is directly or indirectly attached to a C-terminal end of an (Aa) or (Arg/His) residue.

It is to be understood that the payloads may be directly or indirectly attached to the N-terminal end and to the C-terminal end of the peptide linker. In one embodiment, a first payload may be directly attached to the N-terminal amino group of the peptide linker and a second payload may be directly attached to the C-terminal carboxyl group of the peptide linker.

It is, however, preferred that the payloads are indirectly attached to the N-terminal end and to the C-terminal end of the peptide linker, for example with any one of the chemical linkers described herein. In particular, a payload may be indirectly attached to the N-terminal end of the peptide linker via a dicarboxylic acid and a second peptide moiety, as described in more detail elsewhere herein. Furthermore, it is preferred that all payloads are attached to the peptide linker or the chemical linker via a self-immolative moiety, such as any one of the self-immolative moiety disclosed herein.

While the linker comprised in the ADC according to the invention is preferably a peptide linker, as described in detail herein, the linker may as well be any chemical linker, as long as the linker comprises, at least, a cell-permeable topoisomerase I inhibitor and a non-cell-permeable topoisomerase I inhibitor. Moreover, the chemical linker may comprise any of the cleavable moieties, in particular any of the self-immolative moieties, described herein.

As already explained above, the present invention relates to an ADC, i.e., an antibody-linker conjugate comprising any of the linkers defined herein.

That is, the linker comprising at least a cell-permeable topoisomerase I inhibitor and a non-cell-permeable topoisomerase I inhibitor may be conjugated to an antibody by any suitable conjugation method, at any suitable conjugation site.

As described herein above, the ADC of the invention has the formula A-L, wherein A is an antibody or an antibody fragment, and L is a linker. The skilled person is aware that antibodies typically comprise two identical light chains and two identical heavy chains. Consequently, any potential conjugation site in an antibody is typically present in both chains. Thus, the skilled person would understand that ADCs having the formula A-L also encompass ADCs having the formula L-A-L (or A-(L)₂), where one linker is conjugated to a corresponding position in each antibody chain.

Thus, in an alternative embodiment, the invention relates to an antibody-drug conjugate (ADC) having the formula L-A-L, wherein A is an antibody or an antibody fragment and wherein each L is a linker, said linker comprising:
as a first payload a topoisomerase I inhibitor which is cell-permeable, preferably a camptothecin cytotoxic molecule which is cell-permeable; and
as a second payload a topoisomerase I inhibitor which is not cell-permeable, preferably a camptothecin cytotoxic molecule which is not cell-permeable.

In such embodiments, the antibody is preferably a native or engineered full-length antibody comprising two heavy chains and two light chains, even more preferably a full length IgG antibody, as described elsewhere herein.

However, encompassed herein are also ADCs comprising only a single conjugation site. Such ADCs may comprise, without limitation, single chain antibody fragments or engineered antibodies having only a single conjugation site. In such embodiments, the ADC may be conjugated with only a single linker and thus have the formula A-L.

To encompass embodiments where the antibody is conjugated to one or more linker, the ADC may be defined to comprise the formula A-L. Accordingly, in a particular embodiment, the invention relates to an antibody-drug conjugate (ADC) comprising the formula A-L, wherein A is an antibody or an antibody fragment and wherein L is a linker, said linker comprising:
as a first payload a topoisomerase I inhibitor which is cell-permeable, preferably a camptothecin cytotoxic molecule which is cell-permeable; and
as a second payload a topoisomerase I inhibitor which is not cell-permeable, preferably a camptothecin cytotoxic molecule which is not cell-permeable.

It is preferred herein that the amine comprising peptide linkers according to the invention are conjugated to a glutamine residue in an antibody. Such conjugation can be achieved with a transglutaminase, as described in more detail elsewhere herein.

As such, in a particular embodiment, the invention relates to the ADC/antibody-payload conjugate according to the invention, wherein the peptide linker is conjugated to the antibody via an isopeptide bond formed between a γ-carboxamide group of a glutamine residue comprised in the antibody and the primary amine comprised in an amino acid residue of the peptide linker.

Accordingly, the skilled person would readily understand that the primary amine comprised in the peptide linker disclosed herein is no longer present once the peptide linker has been conjugated to the antibody.

The term “antibody” has already been defined above.

In a particular embodiment, the invention relates to an ADC/antibody-payload conjugate according to the invention, wherein the peptide linker is conjugated to a glutamine residue comprised in an Fc domain of the antibody.

That is, the peptide linker according to the invention is preferably conjugated to a glutamine residue comprised in an Fc domain of an antibody. The linkers of the invention may be conjugated to any Gln residue in the Fc domain of an antibody that can serve as a substrate for a transglutaminase. Typically, the term Fc domain as used herein refers to the last two constant region immunoglobulin domains of IgA, IgD and IgG (C_{H}2 and C_{H}3) and the last three constant region domains of IgE, IgY and IgM (C_{H}2, C_{H}3 and C_{H}4). That is, the linker according to the invention may be conjugated to the C_{H}2, C_{H}3 and, where applicable, C_{H}4 domains of the antibody.

For example, the peptide linker according to the invention may be conjugated to an endogenous glutamine residue (e.g., Q295 of an IgG1 antibody) or to a glutamine residue that has been introduced into the Fc domain of the antibody be genetic engineering.

That is, in a particular antibody, the invention relates to an ADC/antibody-payload conjugate according to the invention, wherein the glutamine residue to which the peptide linker is conjugated is glutamine residue Q295 (EU numbering) of the C_{H}2 domain of an IgG antibody.

It is important to understand that Q295 is an extremely conserved amino acid residue in IgG type antibodies. It is conserved in human IgG1, 2, 3, 4, as well as in rabbit and rat antibodies amongst others. Hence, being able to use Q295 is a considerable advantage for making therapeutic antibody-payload conjugates. Even though residue Q295 is extremely conserved among IgG type antibodies, some IgG type antibodies do not possess this residue, such as mouse and rat IgG2a antibodies. Thus, it is to be understood that the antibody used in the method of the present invention is preferably an IgG type antibody comprising residue Q295 (EU numbering) of the C_{H}2 domain.

In the literature discussing the conjugation of linkers to a C_{H}2 Gln residue by means of a transglutaminase, the focus has been on small, low-molecular weight substrates. However, in the prior art literature, to accomplish such conjugation, deglycosylation of the asparagine residue at position N297, or the use of an aglycosylated antibody, has been described as necessary (WO 2015/015448; WO 2017/025179; WO 2013/092998).

Quite surprisingly, and against all expectations, however, site-specific conjugation to Q295 of glycosylated antibodies is indeed efficiently possible by using the above discussed peptide linker structure. In particular, coupling of peptide linkers comprising two or more payloads was achieved with, for most of them, a conjugation efficiency greater than 90%.

Even though Q295 is very close to N297, which is, in its native state, glycosylated, using the specified peptide linker, still allows efficient conjugation to Q295.

The substitution of N297 against another amino acid may have unwanted effects, as it may affect the overall stability of the entire Fc domain (Subedi et al, The Structural Role of Antibody N-Glycosylation in Receptor Interactions. Structure 2015, 23 (9), 1573-1583), and the efficacy of the entire conjugate as a consequence that can lead to increased antibody aggregation and a decreased solubility (Zheng et al.; The impact of glycosylation on monoclonal antibody conformation and stability. Mabs-Austin 2011, 3 (6), 568-576). Further, the glycan that is present at N297 has important immunomodulatory effects, as it triggers antibody dependent cellular cytotoxicity (ADCC) and the like. These immunomodulatory effects would get lost upon deglycosylation or any of the other approaches discussed above to obtain an aglycosylated antibody. Further, any sequence modification of an established antibody can also lead to regulatory problems, which is problematic because very often an accepted and clinically validated antibody is used as a starting point for ADC conjugation.

Hence, in view of the above, in the present invention, for the conjugation of an IgG antibody at residue Q295 (EU numbering) of the C_{H}2 domain of the antibody, preferably, an antibody is used that is glycosylated at residue N297 (EU numbering) of the C_{H}2 domain. However, it is expressly stated that the present invention also encompasses the conjugation of deglycosylated or aglycosylated antibodies at residue Q295 or any other suitable Gln residue of the antibody, wherein the Gln residue may be an endogenous Gln residue or a Gln residue that has been introduced by molecular engineering.

Thus, in a particular embodiment, the invention relates to the antibody-payload conjugate according to the invention, wherein the glutamine residue to which the peptide linker is conjugated has been introduced into the heavy or light chain of the antibody by molecular engineering.

The term “molecular engineering,” as used herein, refers to the use of molecular biology methods to manipulate nucleic acid sequences. In the context of the present invention, molecular engineering may be used to introduce Gln residues into the heavy or light chain of an antibody. In general, two different strategies to introduce Gln residues into the heavy or light chain of an antibody are envisioned within the present invention. First, single residues of the heavy or light chain of an antibody may be substituted with a Gln residue. Second, Gln-containing peptide tags consisting of two or more amino acid residues may be integrated into the heavy or light chain of an antibody. For that, the peptide tag may either be integrated into an internal position of the heavy or light chain, that is, between two existing amino acid residues of the heavy or light chain or by replacing them, or the peptide tag may be fused (appended) to the N- or C-terminal end of the heavy or light chain of the antibody.

For example, an amino residue of the heavy or light chain of an antibody may be substituted with a Gln residue, provided that the resulting antibody can be conjugated with the linkers of the invention by a microbial transglutaminase. In certain embodiments, the antibody is an antibody wherein amino acid residue N297 (EU numbering) of the C_{H}2 domain of an IgG antibody is substituted, in particular wherein the substitution is an N297Q substitution. Antibodies comprising an N297Q mutation may be conjugated to more than one linker per heavy chain of the antibody. For example, antibodies comprising an N297Q mutation may be conjugated to four linkers, wherein one linker is conjugated to residue Q295 of the first heavy chain of the antibody, one linker is conjugated to residue N297Q of the first heavy chain of the antibody, one linker is conjugated to residue Q295 of the second heavy chain of the antibody and one linker is conjugated to residue N297Q of the second heavy chain of the antibody. The skilled person is aware that replacement of residue N297 of an IgG antibody with a Gln residue results in an aglycosylated antibody.

That is, in a particular embodiment, the invention relates to the antibody-payload conjugate according to the invention, wherein the glutamine residue that has been introduced into the heavy or light chain of the antibody by molecular engineering is N297Q (EU numbering) of the CH2 domain of an aglycosylated IgG antibody.

In a particular embodiment, the invention relates to an ADC/antibody-payload conjugate according to the invention, wherein the glutamine residue that has been introduced into the heavy or light chain of the antibody by molecular engineering is comprised in a peptide that has been (a) integrated into the heavy or light chain of the antibody or (b) fused to the N- or C-terminal end of the heavy or light chain of the antibody.

Instead of substituting single amino acid residues of an antibody, peptide tags comprising a Gln residue that is accessible for a transglutaminase may be introduced into the heavy or light chain of the antibody. Such peptide tags may be fused to the N- or C-terminus of the heavy or light chain of the antibody. Alternatively, peptide tags may be inserted into the heavy or light chain of an antibody at a suitable position. Preferably, peptide tags comprising a transglutaminase-accessible Gln residue are fused to the C-terminus of the heavy chain of the antibody. Even more preferably, the peptide tags comprising a transglutaminase-accessible Gln residue are fused to the C-terminus of the heavy chain of an IgG antibody. Several peptide tags that may be fused to the C-terminus of the heavy chain of an antibody and serve as substrate for a microbial transglutaminase are described in WO 2012/059882 and WO 2016/144608.

Thus, in a particular embodiment, the invention relates to the antibody-payload conjugate according to the invention, wherein the peptide comprising the Gln residue has been fused to the C-terminal end of the heavy chain of the antibody.

Exemplary peptide tags that may be introduced into the heavy or light chain of an antibody, in particular fused to the C-terminus of the heavy chain of the antibody, are LLQGG (SEQ ID NO:70), LLQG (SEQ ID NO:37), LSLSQG (SEQ ID NO:38), GGGLLQGG (SEQ ID NO:39), GLLQG (SEQ ID NO:40), LLQ(SEQ ID NO:41), GSPLAQSHGG (SEQ ID NO:42), GLLQGGG (SEQ ID NO:43), GLLQGG (SEQ ID NO:44), GLLQ (SEQ ID NO:45), LLQLLQGA (SEQ ID NO:46), LLQGA(SEQ ID NO:47), LLQYQGA (SEQ ID NO:48), LLQGSG (SEQ ID NO:49), LLQYQG (SEQ ID NO:50), LLQLLQG (SEQ ID NO:51), SLLQG (SEQ ID NO:52), LLQLQ (SEQ ID NO:53), LLQLLQ (SEQ ID NO:54), LLQGR (SEQ ID NO:55), EEQYASTY (SEQ ID NO:56), EEQYQSTY (SEQ ID NO:57), EEQYNSTY (SEQ ID NO:58), EEQYQS (SEQ ID NO:59), EEQYQST (SEQ ID NO:60), EQYQSTY (SEQ ID NO:61), QYQS (SEQ ID NO:62), QYQSTY (SEQ ID NO:63), YRYRQ. (SEQ ID NO:64), DYALQ (SEQ ID NO:65), FGLQRPY (SEQ ID NO:66), EQKLSEEDL (SEQ ID NO:67), LQR (SEQ ID NO:68) and YQR (SEQ ID NO:69).

The skilled person is aware of methods to substitute amino acid residues of antibodies or to introduce peptide tags into antibodies, for example by methods of molecular cloning as described in Sambrook, Joseph. (2001). Molecular cloning: a laboratory manual. Cold Spring Harbor, N.Y.: Cold Spring Harbor Laboratory Press.

While it is preferred herein that antibodies are conjugated at a glutamine reside by means of a transglutaminase, as decribed herein above, the antibody may also be conjugated at a lysine residue. The lysine residue may be an endogenous lysine residue or a lysine residue that has been introduced into the antibody by genetic engineering. In such embodiments, the peptide linker may comprise a glutamine residue that can form an isopeptide bond with the lysine residue of the antibody. Conjugation may be catalyzed by a transglutaminase, as described herein.

In general, the skilled person is aware of methods to determine at which position of an antibody a peptide linker is conjugated. For example, the conjugation site may be determined by proteolytic digestion of the antibody-payload conjugate and LC-MS analysis of the resulting fragments. For example, samples may be deglycosylated with GlyciNATOR (Genovis) according to the instruction manual and subsequently digested with trypsin gold (mass spectrometry grade, Promega), respectively. Therefore, 1 μg of protein may be incubated with 50 ng trypsin at 37° C. overnight. LC-MS analysis may be performed using a nanoAcquity HPLC system coupled to a Synapt-G2 mass spectrometer (Waters). For that, 100 ng peptide solution may be loaded onto an Acquity UPLC Symmetry C18 trap column (Waters, part no. 186006527) and trapped with 5 μL/min flow rate at 1% buffer A (Water, 0.1% formic acid) and 99% buffer B (acetonitrile, 0.1% formic acid) for 3 min. Peptides may then be eluted with a linear gradient from 3% to 65% Buffer B within 25 min. Data may be acquired in resolution mode with positive polarity and in a mass range from 50 to 2000 m/z. Other instrument settings may be as follows: capillary voltage 3.2 kV, sampling cone 40 V, extraction cone 4.0 V, source temperature 130° C., cone gas 35 L/h, nano flow gas 0.1 bar, and purge gas 150 L/h. The mass spectrometer may be calibrated with [Glu1]-Fibrinopeptide.

Further, the skilled person is aware of methods to determine the drug-to-antibody (DAR) ratio or payload-to-antibody ratio of an antibody-payload construct. For example, the DAR may be determined by hydrophobic interaction chromatography (HIC) or LC-MS.

For hydrophobic interaction chromatography (HIC), samples may be adjusted to 0.5 M ammonium sulfate and assessed via a MAB PAK HIC Butyl column (5 μm, 4.6×100 mm, Thermo Scientific) using a full gradient from A (1.5 M ammonium sulfate, 25 mM Tris HCl, pH 7.5) to B (20% isopropanol, 25 mM Tris HCl, pH 7.5) over 20 min at 1 mL/min and 30° C. Typically, 40 μg sample may be used and signals may be recorded at 280 nm. Relative HIC retention times (HIC-RRT) may be calculated by dividing the absolute retention time of the ADC DAR 2 species by the retention time of the respective unconjugated mAb.

For LC-MS DAR determination, ADCs may be diluted with NH₄HCO₃ to a final concentration of 0.025 mg/mL. Subsequently, 40 μL of this solution may be reduced with 1 μL TCEP (500 mM) for 5 min at room temperature and then alkylated by adding 10 μL chloroacetamide (200 mM), followed by overnight incubation at 37° C. in the dark. For reversed phase chromatography, a Dionex U3000 system in combination with the software Chromeleon may be used. The system may be equipped with a RP-1000 column (1000 Å, 5 μm, 1.0×100 mm, Sepax) heated to 70° C., and an UV-detector set to a wavelength of 214 nm. Solvent A may consist of water with 0.1% formic acid and solvent B may comprise 85% acetonitrile with 0.1% formic acid. The reduced and alkylated sample may be loaded onto the column and separated by a gradient from 30-55% solvent B over the course of 14 min. The liquid chromatography system may be coupled to a Synapt-G2 mass spectrometer for identification of the DAR species. The capillary voltage of the mass spectrometer may be set to 3 kV, the sampling cone to 30 V and the extraction cone may add up to a value of 5 V. The source temperature may be set to 150° C., the desolvation temperature to 500° C., the cone gas to 20 l/h, the desolvation gas to 600 l/h, and the acquisition may be made in positive mode in a mass range from 600-5000 Da with 1 s scan time. The instrument may be calibrated with sodium iodide. Deconvolution of the spectra may be performed with the MaxEnt1 algorithm of MassLynx until convergence. After assignment of the DAR species to the chromatographic peaks, the DAR may be calculated based on the integrated peak areas of the reversed phase chromatogram.

In a particular embodiment, the invention relates to the ADC/antibody-payload conjugate of the present invention, wherein the IgG antibody is a glycosylated IgG antibody.

That is, it is preferred herein that the peptide linker according to the invention is conjugated to a glycosylated IgG antibody. It is particularly preferred that the peptide linker according to the invention is conjugated to a native glycosylated IgG antibody. Native IgG antibodies comprise a single conjugation site at glutamine residue 295 (Q295). Thus, it is particularly preferred herein that the peptide linker according to the invention is conjugated to residue Q295 of a native glycosylated antibody. The only glycosylation site of native IgG antibodies is asparagine residue 297 (N297).

Thus, in a particular embodiment, the invention relates to the ADC/antibody-payload conjugate according to the invention, wherein the IgG antibody is glycosylated at residue N297 (EU numbering) of the CH2 domain.

In a particularly preferred embodiment, the peptide linker according to the invention is conjugated to position Q295 of an IgG antibody that is glycosylated at position N297. More preferably, the antibody is an IgG1 antibody.

In a preferred embodiment, the present invention relates to an ADC as defined herein, wherein the linker is a peptide linker.

In a preferred embodiment, the present invention relates to an ADC as defined herein, wherein the first payload and/or second payload are linked to the N- or C-terminus of the peptide linker or to a side-chain of an amino acid residue comprised in the peptide linker; preferably
wherein the first payload is linked to the N-terminus of the peptide linker and wherein the second payload is linked to the C-terminus of the peptide linker, or vice versa.

In a preferred embodiment, the present invention relates to an ADC as defined herein, wherein the linker consists or comprises the following structure (in N->C direction):

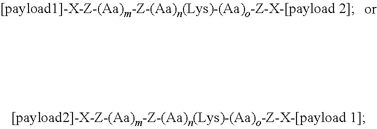

wherein
[payload 1] is said first payload;
[payload 2] is said second payload;
(Aa) is any amino acid residue;
m, n and o may be integers ranging from 0 to 10, preferably 0 to 6, more preferably 0 to 4;
(Lys) is a lysine residue, a lysine mimetic or a lysine derivative;
Z is either absent or a spacer comprising an alkyl or a heteroalkyl group, preferably wherein the spacer is (CH2)₂; and
X is either absent or a self-immolative group, preferably PABC.

More preferably, the present invention relates to an ADC as defined herein, wherein the linker consists of or comprises the following structure:

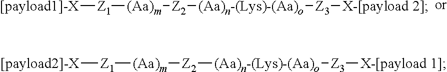

wherein
[payload 1] is said first payload;
[payload 2] is said second payload;
(Aa) is any amino acid residue;
m, n and o may be integers ranging from 0 to 10, preferably 0 to 6, more preferably 0 to 4;
(Lys) is a lysine residue, a lysine mimetic or a lysine derivative;
Z₁₋₃ is either absent or a spacer comprising an alkyl or a heteroalkyl group, preferably wherein the spacer comprises (CH2)₂; and
X is either absent or a self-immolative group, preferably PABC.

In a further preferred embodiment, the residues (Aa)ₘ+(Aa)ₙ+(Aa)ₒ are >0. Even more preferably, the residues (Aa)ₙ+(Aa)ₒ are >0. That is, it is preferred herein that the Lys residue form a peptide bind with at least one additional amino acid reisdue.

In a further preferred embodiment, the spacer Z₂ is a dicarboxylic acid, as disclosed elsewehere herein. This dicarboxylic acid links the N-terminal end of the peptide comprising the (Lys) residue to the N-terminal end of the amino acid or peptide moiety (Aa)ₘ. By linking these two moiteies via their N-terminal ends, a peptide linker comprising two C-terminal ends is formed. These two C-terminal ends can be directly or indirectly linked to payloads, as disclosed elsewhere herein. In such embodiments, it is to be understood that (Aa)ₘ has to be >0.

Accordingly, in a particular embodiment, the invention relates to the ADC according to the invention, wherein Z₂ is a dicarboxylic acid linking the N-terminal end of (Aa)ₘ to the N-terminal end of (Aa)ₙ or (Lys); and wherein one payload is directly or indirectly linked to the C-terminal end of (Aa)ₘ and the other payload is directly or indirectly linked to the C-terminal end of (Lys) or (Aa)ₒ, or vice versa.

In a more preferred embodiment, the present invention relates to an ADC as defined herein, wherein the linker consists of or comprises the following structure:

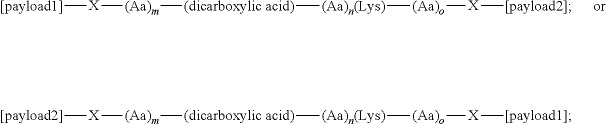

wherein
[payload 1] is said first payload;
[payload 2] is said second payload;
(Aa) is any amino acid residue;
m is an integer ranging from 1 to 10, preferably 1 to 6, more preferably 1 to 4;
n and o may be integers ranging from 0 to 10, preferably 0 to 6, more preferably 0 to 4, wherein n+o is >0;
(Lys) is a lysine residue, a lysine mimetic or a lysine derivative;
(dicarboxylic acid) is dicarboxylic acid linking the N-terminal end of (Aa)ₘ to the N-terminal end of (Aa)ₙ or (Lys); and
X is either absent or a self-immolative group, preferably PABC.

As discussed herein above, the linker preferably comprises at least one positively charged amino acid residue in addition to the lysine residue. In a particularly preferred embodiment, the invention relates to the ADC according to the invention, wherein (Aa)ₙ-(Lys)-(Aa)ₒ comprises the sequence motif Arg-Lys (RK) or His-Lys (HK) (in N->C direction). In another preferred embodiment, the invention relates to the ADC according to the invention, wherein (Aa)ₙ-(Lys)-(Aa)ₒ is or comprises RK or RKAA (in N->C direction).

In such embodiments, the moiety (Aa)ₘ is preferably a single amino acid residue or a peptide having a length of 2-10, preferably 2-6, more preferably 2-4 amino acid residues. In certain embodiments, the moiety (Aa)ₘ has or comprises the sequence Ala-Ala (AA) or Ala-Arg-Ala (ARA).

As regards the definitions of this ADC as well as preferred embodiments thereof, same applies, mutatis mutandis, as has been set forth above in the context of the ADC of the present invention.

In a preferred embodiment, the present invention relates to an ADC as defined herein, wherein the linker comprises or consists of the following structure:

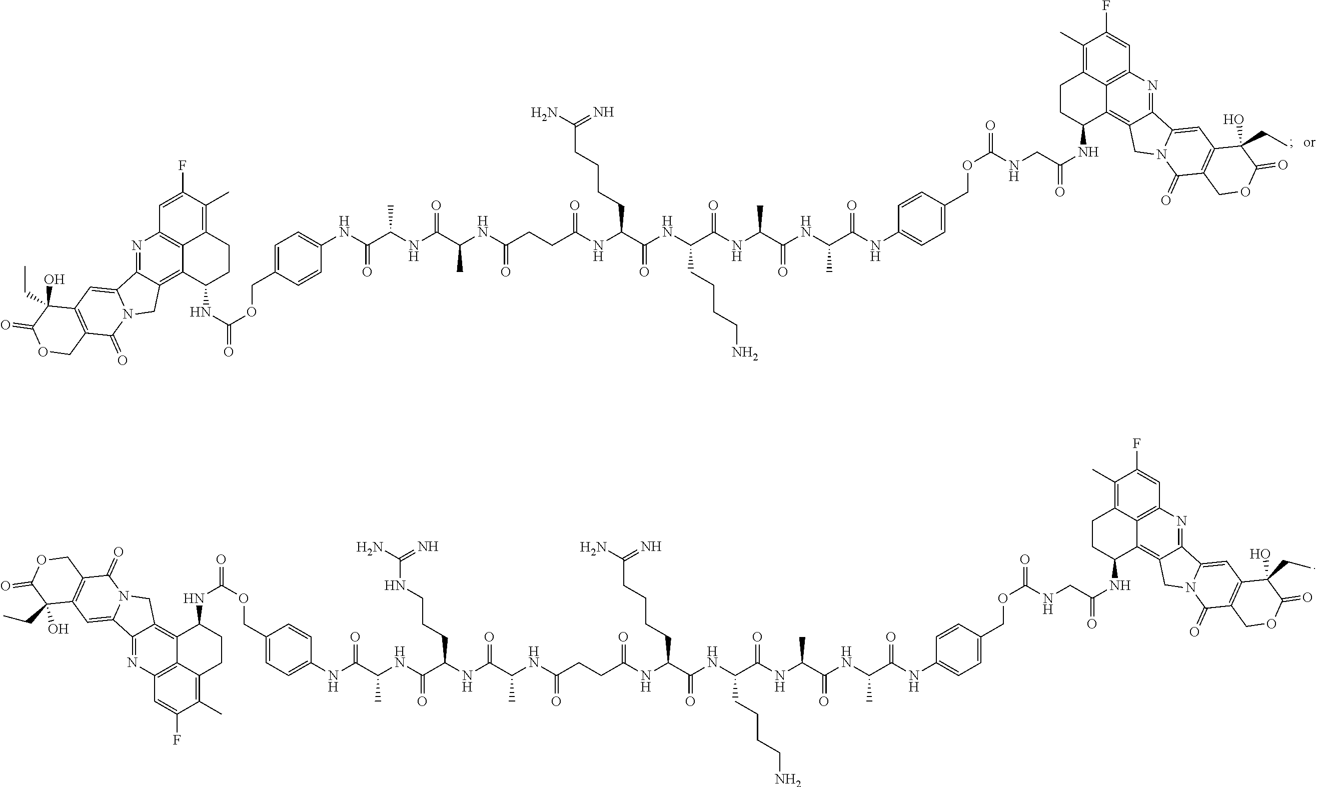

In a preferred embodiment, the present invention relates to an ADC as defined herein, wherein said ADC comprises more than one first payloads and/or more than one second payloads.

As already outlined above, the peptide linker in accordance with the present invention comprises or contains at least two payloads.

If the peptide linker comprises or contains only two payloads, these two payloads are different in structure, i.e., the first payload is a camptothecin cytotoxic molecule which is cell-permeable; and the second payload is a camptothecin cytotoxic molecule which is not cell-permeable.

If the peptide linker comprises or contains more than two payloads, the payloads may be identical or may be different in structure while at least two of these payloads may be identical in structure.

Coupling two or more identical payloads to a peptide linker allows increasing the concentration of the payload in the target tissue or cell of an antibody-payload conjugate. For example, if the peptide linker of an antibody-payload conjugate comprises two or more identical toxins (resulting in a DAR>4 ADC), the concentration of the toxin in the target tissue or cell will be higher compared to a conventional DAR2 ADC. With the peptide linkers of the present invention, ADCs comprising 4, 6 or 8 identical payload molecules may be obtained.

In a particular embodiment, the peptide linker in accordance with the present invention comprises between 2 and 4 payloads.

In a particular embodiment, the peptide linker in accordance with the present invention comprises or contains 2 payloads, i.e., a as a first payload a single topoisomerase I inhibitor which is cell-permeable; and as a second payload a single topoisomerase I inhibitor which is not cell-permeable.

In a particular embodiment, the peptide linker in accordance with the present invention comprises or contains 3 payloads, i.e., as a first payload two topoisomerase I inhibitors which are cell-permeable; and as a second payload a single topoisomerase I inhibitor which is not cell-permeable.

In a particular embodiment, the peptide linker in accordance with the present invention comprises or contains 3 payloads, i.e., as a first payload a single topoisomerase I inhibitor which is cell-permeable; and as a second payload two topoisomerase I inhibitors which are not cell-permeable.

In a particular embodiment, the peptide linker in accordance with the present invention comprises or contains 4 payloads, i.e., as a first payload a single topoisomerase I inhibitor which is cell-permeable; and as a second payload three topoisomerase I inhibitors which are not cell-permeable.

In a particular embodiment, the peptide linker in accordance with the present invention comprises or contains 4 payloads, i.e., as a first payload two topoisomerase I inhibitors which are cell-permeable; and as a second payload two topoisomerase I inhibitors which are not cell-permeable.

In a particular embodiment, the peptide linker in accordance with the present invention comprises or contains 4 payloads, i.e., as a first payload three topoisomerase I inhibitors which are cell-permeable; and as a second payload a single topoisomerase I inhibitor which is not cell-permeable.

In a preferred particular embodiment, the peptide linker in accordance with the present invention comprises or contains 2 payloads, i.e., a as a first payload a single camptothecin cytotoxic molecule which is cell-permeable; and as a second payload a single camptothecin cytotoxic molecule which is not cell-permeable.

In a preferred particular embodiment, the peptide linker in accordance with the present invention comprises or contains 3 payloads, i.e., as a first payload two camptothecin cytotoxic molecules which are cell-permeable; and as a second payload a single camptothecin cytotoxic molecule which is not cell-permeable.

In a preferred particular embodiment, the peptide linker in accordance with the present invention comprises or contains 3 payloads, i.e., as a first payload a single camptothecin cytotoxic molecule which is cell-permeable; and as a second payload two camptothecin cytotoxic molecules which are not cell-permeable.

In a preferred particular embodiment, the peptide linker in accordance with the present invention comprises or contains 4 payloads, i.e., as a first payload a single camptothecin cytotoxic molecule which is cell-permeable; and as a second payload three camptothecin cytotoxic molecules which are not cell-permeable.

In a preferred particular embodiment, the peptide linker in accordance with the present invention comprises or contains 4 payloads, i.e., as a first payload two camptothecin cytotoxic molecules which are cell-permeable; and as a second payload two camptothecin cytotoxic molecules which are not cell-permeable.

In a preferred particular embodiment, the peptide linker in accordance with the present invention comprises or contains 4 payloads, i.e., as a first payload three camptothecin cytotoxic molecules which are cell-permeable; and as a second payload a single camptothecin cytotoxic molecule which is not cell-permeable.

Linkers comprising three payloads may have the following structure:

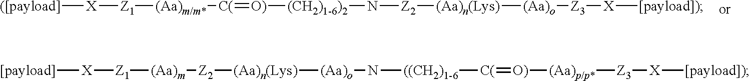

wherein
[payload] is each independently a payload selected from the first and second payload, wherein the linker comprises at least one of each the first and second payload;
(Aa) is any amino acid residue;
m, m*, n, o, p and p* may be integers ranging from 0 to 10, preferably 0 to 6, more preferably 0 to 4, even more preferably wherein n+o is >0;
(Lys) is a lysine residue, a lysine mimetic or a lysine derivative;
Z₁₋₃ is either absent or a spacer comprising an alkyl or a heteroalkyl group, preferably wherein the spacer comprises (CH₂)₂, even more preferably wherein Z₂ is a dicarboxylic acid linker; and
X is either absent or a self-immolative group, preferably PABC.

More preferably, linkers comprising three payloads may have the following structure:

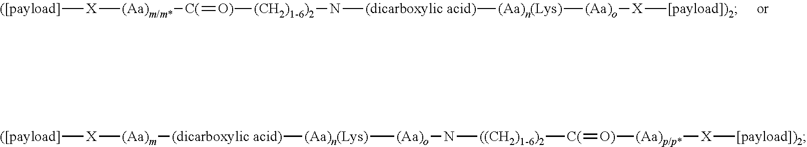

wherein
[payload] is each independently a payload selected from the first and second payload, wherein the linker comprises at least one of each the first and second payload;
(Aa) is any amino acid residue;
m, m*, p and p* are integers ranging from 1 to 10, preferably 1 to 6, more preferably 1 to 4;
n and o may be integers ranging from 0 to 10, preferably 0 to 6, more preferably 0 to 4, wherein n+o is >0;
(Lys) is a lysine residue, a lysine mimetic or a lysine derivative;
(dicarboxylic acid) is dicarboxylic acid linking the N-terminal end of (Aa)ₘ or the disubstituted amine (N) to the N-terminal end of (Aa)ₙ or (Lys); and
X is either absent or a self-immolative group, preferably PABC.

Linkers comprising four payloads may have the following structure:

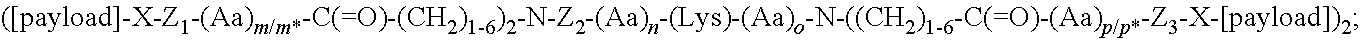

wherein
[payload] is each independently a payload selected from the first and second payload, wherein the linker comprises at least one of each the first and second payload;
(Aa) is any amino acid residue;
m, m*, n, o, p and p* may be integers ranging from 0 to 10, preferably 0 to 6, more preferably 0 to 4, even more preferably wherein n+o is >0;
(Lys) is a lysine residue, a lysine mimetic or a lysine derivative;
Z₁₋₃ is either absent or a spacer comprising an alkyl or a heteroalkyl group, preferably wherein the spacer comprises (CH₂)₂, even more preferably wherein Z₂ is a dicarboxylic acid linker; and
X is either absent or a self-immolative group, preferably PABC.

More preferably, linkers comprising four payloads may have the following structure:

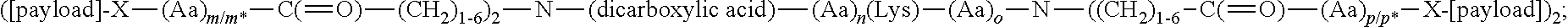

wherein
[payload] is each independently a payload selected from the first and second payload, wherein the linker comprises at least one of each the first and second payload;
(Aa) is any amino acid residue;
m, m*, p and p* are integers ranging from 1 to 10, preferably 1 to 6, more preferably 1 to 4;
n and o may be integers ranging from 0 to 10, preferably 0 to 6, more preferably 0 to 4, wherein n+o is >0;
(Lys) is a lysine residue, a lysine mimetic or a lysine derivative;
(dicarboxylic acid) is dicarboxylic acid as defined herein linking the N-terminal end of (Aa)ₘ or the disubstituted amine (N) to the N-terminal end of (Aa)ₙ or (Lys); and
X is either absent or a self-immolative group, preferably PABC.

The linkers preferably comprise at least one additional positively charged amino acid residue in addition to the lysine residue, as explained in detail elsewhere herein.

In a preferred embodiment, the present invention relates to an ADC as defined herein, wherein the camptothecin is an exatecan or an extecan derivative.

As already outlined above, the cytotoxic molecule of the ADC of the present invention is a topoisomerase I inhibitor, preferably a camptothecin. While numerous camptothecins are known in the art, including, for example, topotecan, exatecan, irinotecan, DX-8951f, SN38, BN 80915, lurtotecan, 9-nitrocamptothecin and aminocamptothecin, preferred camptothecins in accordance with the present invention is an exatecan.

In a preferred embodiment, the present invention relates to an ADC as defined herein, wherein the second payload has an amino acid (preferably a glycine, alanine, arginine, asparagine, aspartic acid, cysteine, glutamine, glutamic acid, histidine, isoleucine, leucine, lysine, methionine, phenylalanine, proline, serine, threonine, tryptophan, tyrosine or valine residue) residue linked to said topoisomerase I inhibitor (preferably, said camptothecin cytotoxic molecule) of the second payload.

Without being bound by theory, said amino acid residue (preferably a glycine, alanine, arginine, asparagine, aspartic acid, cysteine, glutamine, glutamic acid, histidine, isoleucine, leucine, lysine, methionine, phenylalanine, proline, serine, threonine, tryptophan, tyrosine or valine residue) has the function of rendering the second payload not cell-permeable.

Thus, in a preferred embodiment, the present invention relates to an ADC as defined herein, wherein the second payload has an amino acid residue (preferably a glycine, alanine, arginine, asparagine, aspartic acid, cysteine, glutamine, glutamic acid, histidine, isoleucine, leucine, lysine, methionine, phenylalanine, proline, serine, threonine, tryptophan, tyrosine or valine residue) linked to said topoisomerase I inhibitor of the second payload, thereby rendering it not cell-permeable.

Thus, in a preferred embodiment, the present invention relates to an ADC as defined herein, wherein the second payload has an amino acid residue (preferably a glycine, alanine, arginine, asparagine, aspartic acid, cysteine, glutamine, glutamic acid, histidine, isoleucine, leucine, lysine, methionine, phenylalanine, proline, serine, threonine, tryptophan, tyrosine or valine residue) as part of the linker which is linked to said topoisomerase I inhibitor of the second payload, thereby rendering it not cell-permeable upon releasing the corresponding amino acid-cytotoxic construct.

The capability of a molecule (in the present case the topoisomerase I inhibitor) to be cell-permeable or not cell-permeable can be measured/determined by methods known in the art and as described herein.

In a preferred embodiment, the present invention relates to an ADC as defined herein, wherein the second payload has a glycine residue linked to said topoisomerase I inhibitor (preferably, said camptothecin cytotoxic molecule) of the second payload.

Without being bound by theory, said glycine residue has the function of rendering the second payload not cell-permeable.

Thus, in a preferred embodiment, the present invention relates to an ADC as defined herein, wherein the second payload has a glycine residue linked to said topoisomerase I inhibitor of the second payload, thereby rendering it not cell-permeable.

Thus, in a preferred embodiment, the present invention relates to an ADC as defined herein, wherein the second payload has a glycine residue as part of the linker which is linked to said topoisomerase I inhibitor of the second payload, thereby rendering it not cell-permeable upon releasing the glycine-cytotoxic construct.

The capability of a molecule (in the present case the topoisomerase I inhibitor) to be cell-permeable or not cell-permeable can be measured/determined by methods known in the art and as already described herein.

In a preferred embodiment, the present invention relates to an ADC as defined herein, wherein the second payload has an amino acid residue (preferably a glycine, alanine, arginine, asparagine, aspartic acid, cysteine, glutamine, glutamic acid, histidine, isoleucine, leucine, lysine, methionine, phenylalanine, proline, serine, threonine, tryptophan, tyrosine or valine residue) linked to said camptothecin cytotoxic molecule of the second payload.

Without being bound by theory, said amino acid residue (preferably a glycine, alanine, arginine, asparagine, aspartic acid, cysteine, glutamine, glutamic acid, histidine, isoleucine, leucine, lysine, methionine, phenylalanine, proline, serine, threonine, tryptophan, tyrosine or valine residue) has the function of rendering the second payload not cell-permeable.

Thus, in a preferred embodiment, the present invention relates to an ADC as defined herein, wherein the second payload has an amino acid residue (preferably a glycine, alanine, arginine, asparagine, aspartic acid, cysteine, glutamine, glutamic acid, histidine, isoleucine, leucine, lysine, methionine, phenylalanine, proline, serine, threonine, tryptophan, tyrosine or valine residue) linked to said camptothecin cytotoxic molecule of the second payload, thereby rendering it not cell-permeable.

Thus, in a preferred embodiment, the present invention relates to an ADC as defined herein, wherein the second payload has an amino acid residue (preferably a glycine, alanine, arginine, asparagine, aspartic acid, cysteine, glutamine, glutamic acid, histidine, isoleucine, leucine, lysine, methionine, phenylalanine, proline, serine, threonine, tryptophan, tyrosine or valine residue) as part of the linker which is linked to said camptothecin cytotoxic molecule of the second payload, thereby rendering it not cell-permeable upon releasing the corresponding amino acid-cytotoxic construct.

The capability of a molecule (in the present case the camptothecin cytotoxic molecule) to be cell-permeable or not cell-permeable can be measured/determined by methods known in the art and as described herein.

In a preferred embodiment, the present invention relates to an ADC as defined herein, wherein the second payload has a glycine residue linked to said camptothecin cytotoxic molecule of the second payload.

Without being bound by theory, said glycine residue has the function of rendering the second payload not cell-permeable.

Thus, in a preferred embodiment, the present invention relates to an ADC as defined herein, wherein the second payload has a glycine residue linked to said camptothecin cytotoxic molecule of the second payload, thereby rendering it not cell-permeable.

Thus, in a preferred embodiment, the present invention relates to an ADC as defined herein, wherein the second payload has a glycine residue as part of the linker which is linked to said camptothecin cytotoxic molecule of the second payload, thereby rendering it not cell-permeable upon releasing the glycine-cytotoxic construct.

The capability of a molecule (in the present case the camptothecin cytotoxic molecule) to be cell-permeable or not cell-permeable can be measured/determined by methods known in the art and as already described herein.

In a preferred embodiment, the present invention relates to an ADC as defined herein, wherein the antibody is an IgG antibody, in particular an IgG1 antibody.

The terms “IgG antibody” and “IgG1 antibody” have already been described above. As regards these definitions as well as preferred embodiments thereof, same applies, mutatis mutandis, as has been set forth above in the context of the ADC of the present invention.

The present invention is not limited to a specific antibody, and the ADC may comprise any antibody, preferably any antibody that can be used in cancer therapy.

In a preferred embodiment, the present invention relates to an ADC as defined herein, wherein the antibody is selected from the group consisting of: Trastuzumab, Brentuximab, Gemtuzumab, Inotuzumab, Avelumab, Cetuximab, Rituximab, Daratumumab, Pertuzumab, Vedolizumab, Ocrelizumab, Tocilizumab, Ustekinumab, Golimumab, Obinutuzumab, Sacituzumab, Belantamab, Polatuzumab, Enfortumab, Endrecolomab, Gemtuzumab,Loncastuximab, Mecbotamab, Adecatumumab, D93, Gatipotuzumab, Labetuzumab, Tusamitamab, Upifitamab, Lifastuzumab, Mirvetuximab, Sofituzumab, Anetumab, Tisotumab, Cofituzumab, Praluzatamab, Ladriatuzumab, Belantamab, Patritumab, Cetuximab, Nimotuzumab, Matuzumab, Portuzumab, Citatuzumab, Tucotuzumab and Endrecolomab; and/or
wherein the antibody specifically binds to an antigen selected from the group consisting of: CD30, Her2/neuCD33, CD22, PD-L1, EGFR, CD20, CD38, HER2, Integrin α4β7, CD20, IL-6-R, IL-12, IL-23, TNFα, CD20, Trop-2, BCMA, CD79b, Nectin-4, EpCAM, CD33, CD19, AXL, dn-collagen, TA-MUC1, carcinoembryonic cell adhesion molecule 5, CEACAM5, NaPi2b, FRα, MUC16, mesothelin, TF, CD166, LIV-1, ERBB3, EGFR, and TACSTD1, preferably, CD30, Her2/neu, CD22, CD79b, Nectin-4, Trop-2 and BCMA, more preferably, CD79b, Her2/neu, and Nectin-4.

The above antibodies are known in the art. In the following, it is indicated in brackets to which antigen said respective antibody specifically binds to: Trastuzumab (anti-Her2/neu), Brentuximab (anti-CD30), Gemtuzumab (anti-CD33), Inotuzumab (anti-CD22), Avelumab (anti-PD-L1), Cetuximab (anti-EGFR), Rituximab (anti-CD20), Daratumumab (anti-CD38), Pertuzumab (anti-HER2), Vedolizumab (anti-Integrin α4β7), Ocrelizumab (anti-CD20), Tocilizumab (anti-IL-6-R), Ustekinumab (anti-IL-12/23), Golimumab (anti-TNFα), Obinutuzumab (anti-CD20), Sacituzumab (anti-Trop-2), Belantamab (anti-BCMA), Polatuzumab (anti-CD79b), Enfortumab (anti-Nectin-4), Endrecolomab (anti-EpCAM), Gemtuzumab (anti-CD33), Loncastuximab (anti-CD19), Mecbotamab (anti-AXL), Adecatumumab (anti-EpCAM), D93 (anti-dn-collagen), Gatipotuzumab (anti-TA-MUC1), Labetuzumab (anti-carcinoembryonic cell adhesion molecule 5), Tusamitamab (anti-CEACAM5), Upifitamab (anti-NaPi2b), Lifastuzumab (anti-NaPi2b), Mirvetuximab (anti-FRα)), Sofituzumab (anti-MUC16), Anetumab (anti-mesothelin), Tisotumab (anti-TF), Cofituzumab (anti-Trop-2), Praluzatamab (anti-CD166), Ladriatuzumab (anti-LIV-1), Belantamab (anti-BCMA), Patritumab (anti-ERBB3), Cetuximab (anti-EGFR), Nimotuzumab (anti-EGFR), Matuzumab (anti-EGFR), Portuzumab (anti-HER2), Citatuzumab (anti-TACSTD1), Tucotuzumab (anti-EpCAM) and Endrecolomab (anti-EpCAM).

In a particular preferred embodiment, the present invention relates to an ADC as defined herein, wherein the antibody is Trastuzumab (anti-Her2/neu).

In a preferred embodiment, the antibody is Trastuzumab with a heavy chain as set forth in SEQ ID NO:73 and a light chain as set forth in SEQ ID NO:74.

In another embodiment, the antibody is an anti-Her2/neu antibody with a heavy chain variable region as set forth in SEQ ID NO:102 and a light chain variable region as set forth in SEQ ID NO:103.

In another embodiment, the antibody is an anti-Her2/neu antibody with a heavy chain CDR1 (CDR-H1, Kabat) as set forth in SEQ ID NO:104, a heavy chain CDR2 (CDR-H2, Kabat) as set forth in SEQ ID NO:105, a heavy chain CDR3 (CDR-H3, Kabat) as set forth in SEQ ID NO:106, a light chain CDR1 (CDR-L1, Kabat) as set forth in SEQ ID NO:107, a light chain CDR2 (CDR-L2, Kabat) as set forth in SEQ ID NO:108, and a light chain CDR3 (CDR-L3, Kabat) as set forth in SEQ ID NO:109.

In another particular preferred embodiment, the present invention relates to an ADC as defined herein, wherein the antibody is Polatuzumab (anti-CD79b).

In a preferred embodiment, the antibody is Polatuzumab with a heavy chain as set forth in SEQ ID NO:71 and a light chain as set forth in SEQ ID NO:72.

In another embodiment, the antibody is an anti-CD79b antibody with a heavy chain variable region as set forth in SEQ ID NO:110 and a light chain variable region as set forth in SEQ ID NO:111.

In another embodiment, the antibody is an anti-CD79b antibody with a heavy chain CDR1 (CDR-H1, Kabat) as set forth in SEQ ID NO:112, a heavy chain CDR2 (CDR-H2, Kabat) as set forth in SEQ ID NO:113, a heavy chain CDR3 (CDR-H3, Kabat) as set forth in SEQ ID NO:114, a light chain CDR1 (CDR-L1, Kabat) as set forth in SEQ ID NO:115, a light chain CDR2 (CDR-L2, Kabat) as set forth in SEQ ID NO:116, and a light chain CDR3 (CDR-L3, Kabat) as set forth in SEQ ID NO:117.

In another particular preferred embodiment, the present invention relates to an ADC as defined herein, wherein the antibody is Enfortumab (anti-Nectin-4) or a variant thereof.

In a preferred embodiment, the antibody is Enfortumab with a heavy chain as set forth in SEQ ID NO:75 and a light chain as set forth in SEQ ID NO:95, 76 or 77.

As outlined above, in a preferred embodiment, the invention relates to the antibody-payload conjugate according to the invention, wherein the antibody is an antibody, preferably, an IgG antibody comprising at Kabat position 234 an A and/or at Kabat position 235 an A.

Accordingly, in certain embodiments, the antibody is Enfortumab or a variant thereof comprising at Kabat position 234 an A and at Kabat position 235 an A with a heavy chain as set forth in SEQ ID NO:94.

In certain embodiments, the anti-Nectin-4 antibody is Enfortumab comprising at Kabat position 234 an A and at Kabat position 235 an A with a heavy chain as set forth in SEQ ID NO:94 and a light chain as set forth in SEQ ID NO:95 or 76 or 77.

In another embodiment, the antibody is an anti-Nectin-4 antibody with a heavy chain variable region as set forth in SEQ ID NO:118 and a light chain variable region as set forth in SEQ ID NO:119.

In another embodiment, the antibody is an anti-Nectin-4 antibody with a heavy chain CDR1 (CDR-H1, Kabat) as set forth in SEQ ID NO:120, a heavy chain CDR2 (CDR-H2, Kabat) as set forth in SEQ ID NO:121, a heavy chain CDR3 (CDR-H3, Kabat) as set forth in SEQ ID NO:122, a light chain CDR1 (CDR-L1, Kabat) as set forth in SEQ ID NO:123, a light chain CDR2 (CDR-L2, Kabat) as set forth in SEQ ID NO:124 or SEQ ID NO:142, and a light chain CDR3 (CDR-L3, Kabat) as set forth in SEQ ID NO:125.

In another particular preferred embodiment, the present invention relates to an ADC as defined herein, wherein the antibody is m290 (anti-Nectin-4).

In a preferred embodiment, the antibody is m290 with a heavy chain as set forth in SEQ ID NO:96 and a light chain as set forth in SEQ ID NO:97.

In another embodiment, the antibody is an anti-Nectin-4 antibody with a heavy chain variable region as set forth in SEQ ID NO:126 and a light chain variable region as set forth in SEQ ID NO:127.

In another embodiment, the antibody is an anti-Nectin-4 antibody with a heavy chain CDR1 (CDR-H1, Kabat) as set forth in SEQ ID NO:128, a heavy chain CDR2 (CDR-H2, Kabat) as set forth in SEQ ID NO:129, a heavy chain CDR3 (CDR-H3, Kabat) as set forth in SEQ ID NO:130, a light chain CDR1 (CDR-L1, Kabat) as set forth in SEQ ID NO:131, a light chain CDR2 (CDR-L2, Kabat) as set forth in SEQ ID NO:132, and a light chain CDR3 (CDR-L3, Kabat) as set forth in SEQ ID NO:133.

In another particular preferred embodiment, the present invention relates to an ADC as defined herein, wherein the antibody is Upifitamab (anti-NaPi2b).

In a preferred embodiment, the antibody is Upifitamab with a heavy chain as set forth in SEQ ID NO:98 and a light chain as set forth in SEQ ID NO:99.

As outlined above, in a preferred embodiment, the invention relates to the antibody-payload conjugate according to the invention, wherein the antibody is an antibody, preferably, an IgG antibody comprising at Kabat position 234 an A and/or at Kabat position 235 an A.

Accordingly, in certain embodiments, the antibody is Upifitamab comprising at Kabat position 234 an A and at Kabat position 235 an A with a heavy chain as set forth in SEQ ID NO:100.

In certain embodiments, the anti-NaPi2b antibody is Upifitamab comprising at Kabat position 234 an A and at Kabat position 235 an A with a heavy chain as set forth in SEQ ID NO:100 and a light chain as set forth in SEQ ID NO:101.

In another embodiment, the antibody is an anti-NaPi2b antibody with a heavy chain variable region as set forth in SEQ ID NO:134 and a light chain variable region as set forth in SEQ ID NO:135.

In another embodiment, the antibody is an anti-NaPi2b antibody with a heavy chain CDR1 (CDR-H1, Kabat) as set forth in SEQ ID NO:136, a heavy chain CDR2 (CDR-H2, Kabat) as set forth in SEQ ID NO:137, a heavy chain CDR3 (CDR-H3, Kabat) as set forth in SEQ ID NO:138, a light chain CDR1 (CDR-L1, Kabat) as set forth in SEQ ID NO:139, a light chain CDR2 (CDR-L2, Kabat) as set forth in SEQ ID NO:140, and a light chain CDR3 (CDR-L3, Kabat) as set forth in SEQ ID NO:141.

As mentioned, in certain embodiments, the present invention relates to an ADC as defined herein, wherein the antibody specifically binds to an antigen selected from the group consisting of: CD30, Her2/neuCD33, CD22, PD-L1, EGFR, CD20, CD38, HER2, Integrin α4β7, CD20, IL-6-R, IL-12, IL-23, TNFα, CD20, Trop-2, BCMA, CD79b, Nectin-4, EpCAM, CD33, CD19, AXL, dn-collagen, TA-MUC1, carcinoembryonic cell adhesion molecule 5, CEACAM5, NaPi2b, FRα, MUC16, mesothelin, TF, CD166, LIV-1, ERBB3, EGFR, and TACSTD1, preferably, CD30, Her2/neu, CD22, CD79b, Nectin-4, Trop-2 and BCMA, more preferably, CD79b, Her2/neu, and Nectin-4.

In a preferred embodiment, the present invention relates to an ADC as defined herein, wherein said ADC consists of two first payloads and two second payloads (drug-to-antibody ratio of 4 “DAR4”).

Accordingly, in this embodiment,
the peptide linker in accordance with the present invention comprises or contains 2 payloads, i.e., as a first payload a single topoisomerase I inhibitor which is cell-permeable; and as a second payload a single topoisomerase I inhibitors which are not cell-permeable.

Accordingly, in a preferred embodiment,
the peptide linker in accordance with the present invention comprises or contains 2 payloads, i.e., as a first payload a single camptothecin cytotoxic molecule which is cell-permeable; and as a second payload a single camptothecin cytotoxic molecules which is not cell-permeable.

In a preferred embodiment, the present invention relates to an ADC as defined herein, wherein said ADC consists of two first payloads and four second payloads (drug-to-antibody ratio of 6 “DAR6”).

Accordingly, in this embodiment,
the peptide linker in accordance with the present invention comprises or contains 3 payloads, i.e., as a first payload a single topoisomerase I inhibitor which is cell-permeable; and as a second payload two topoisomerase I inhibitors which are not cell-permeable.

Accordingly, in a preferred embodiment,
the peptide linker in accordance with the present invention comprises or contains 3 payloads, i.e., as a first payload a single camptothecin cytotoxic molecule which is cell-permeable; and as a second payload two camptothecin cytotoxic molecules which are not cell-permeable

In a preferred embodiment, the present invention relates to an ADC as defined herein, wherein said ADC consists of four first payloads and two second payloads (drug-to-antibody ratio of 6 “DAR6”).

Accordingly, in this embodiment,
the peptide linker in accordance with the present invention comprises or contains 3 payloads, i.e., as a first payload two topoisomerase I inhibitors which are cell-permeable; and as a second payload a single topoisomerase I inhibitors which is not cell-permeable.

Accordingly, in a preferred embodiment,
the peptide linker in accordance with the present invention comprises or contains 3 payloads, i.e., as a first payload two camptothecin cytotoxic molecules which are cell-permeable; and as a second payload a single camptothecin cytotoxic molecules which are not cell-permeable.

In a preferred embodiment, the present invention relates to an ADC as defined herein, wherein said ADC consists of four first payloads and four second payloads (drug-to-antibody ratio of 8 “DAR8”).

Accordingly, in this embodiment,
the peptide linker in accordance with the present invention comprises or contains 4 payloads, i.e., as a first payload two topoisomerase I inhibitor which are cell-permeable; and as a second payload two topoisomerase I inhibitors which are not cell-permeable.

Accordingly, in a preferred embodiment,
the peptide linker in accordance with the present invention comprises or contains 4 payloads, i.e., as a first payload two camptothecin cytotoxic molecule which are cell-permeable; and as a second payload two camptothecin cytotoxic molecules which are not cell-permeable.

In a preferred embodiment, the present invention relates to an ADC as defined herein, wherein said ADC consists of two first payloads and six second payloads (drug-to-antibody ratio of 8 “DAR8”).

Accordingly, in this embodiment,
the peptide linker in accordance with the present invention comprises or contains 4 payloads, i.e., as a first payload a single topoisomerase I inhibitors which is cell-permeable; and as a second payload three topoisomerase I inhibitors which are not cell-permeable.

Accordingly, in a preferred embodiment,
the peptide linker in accordance with the present invention comprises or contains 4 payloads, i.e., as a first payload a single camptothecin cytotoxic molecules which are cell-permeable; and as a second payload three camptothecin cytotoxic molecules which are not cell-permeable.

In a preferred embodiment, the present invention relates to an ADC as defined herein, wherein said ADC consists of six first payloads and two second payloads (drug-to-antibody ratio of 8 “DAR8”).

Accordingly, in this embodiment,
the peptide linker in accordance with the present invention comprises or contains 4 payloads, i.e., as a first payload three topoisomerase I inhibitors which are cell-permeable; and as a second payload a single topoisomerase I inhibitor which is not cell-permeable.

Accordingly, in a preferred embodiment,
the peptide linker in accordance with the present invention comprises or contains 4 payloads, i.e., as a first payload three camptothecin cytotoxic molecules which are cell-permeable; and as a second payload a single camptothecin cytotoxic molecule which is not cell-permeable.

In a further aspect of the present invention, the present invention relates to an ADC as defined herein above, wherein the linker further comprises a third payload, preferably, a toxin or a cytotoxin, more preferably, an auristatin, and even more preferably, an MMAE (Monomethyl auristatin E).

This further aspect is connected to the above-defined ADC of the present invention and is described in more detail in the following.

While the first and second payload, respectively, as defined herein-above is defined to be a topoisomerase inhibibor I in terms of the present invention, in the context of the further aspect, there is a third payload. This third payload of the further aspect of the present invention is different in structure compared to the first and second payload.

Accordingly, in a preferred embodiment, this third payload is not a topoisomerase I inhibitor which is cell-permeable and is not a topoisomerase inhibitor I which is not cell permeable as defined herein above.

In a more preferred embodiment, the third payload is not a camptothecin cytotoxic molecule which is cell-permeable; and is not a camptothecin cytotoxic molecule which is not cell-permeable.

As regards preferred embodiments of the terms “topoisomerase I inhibitor which is cell-permeable”, “topoisomerase inhibitor I which is not cell permeable”, “camptothecin cytotoxic molecule which is cell-permeable” and “camptothecin cytotoxic molecule which is not cell-permeable”, the same applies, mutatis mutandis, to this further aspect of the present invention (with the exception that the respective feature is not the third payload).

In a preferred embodiment, the present invention in accordance with said further aspect of the present invention relates to an ADC as defined herein above, wherein the linker further comprises a third payload, wherein said third payload is a toxin, cytotoxin or a cytotoxic molecule.

The terms “toxin”, “cytotoxin” and “cytotoxic molecule” have already been defined above.

In a particular embodiment, in the context of the further aspect, the present invention relates to an ADC as defined herein, wherein said linker further comprises a third payload, wherein the third payload is at least one selected from the group consisting of:
a pyrrolobenzodiazepine (e.g., PBD);
an auristatin (e.g., MMAE, MMAF);
a maytansinoid (e.g., maytansine, DM1, DM4, DM21);
a duocarmycin;
a nicotinamide phosphoribosyltransferase (NAMPT) inhibitor;
a tubulysin;
an enediyne (e.g., calicheamicin);
an anthracycline derivative (PNU) (e.g., doxorubicin);
a pyrrole-based kinesin spindle protein (KSP) inhibitor;
a cryptophycin;
a drug efflux pump inhibitor;
a sandramycin;
an antimetabolite or metabolite inhibitor, preferably a thymidylate synthase inhibitor;
an amanitin (e.g., α-amanitin);
a DNA damage repair inhibitor; and
a topoisomerase II inhibitor.

That is, in the context of the further aspect, the ADC, preferably, the linker of the invention preferably comprises a toxin as a third payload. The term “toxin” as used herein relates to any compound that is poisonous to a cell or organism. The terms “poison” or “poisonous” are commonly known in the art and refer in general terms to a substance that is harmful or lethal to a living organism in terms that it causes death, injury or harm to an organism or cell and/or triggers apoptosis of the cells. Preferably, the toxin is produced by a cell or an organism. However, the toxin may also be a chemical derivative or analog of a toxin that is produced by a cell or an organism. Toxins can be, without limitation, small molecules, peptides, or proteins. Specific examples are neurotoxins, necrotoxins, hemotoxins and cytotoxins. In certain embodiments, the toxin is a toxin that is used in the treatment of neoplastic diseases. That is, the toxin may be conjugated to an antibody with the method of the invention and delivered to or into a malignant cell due to the target specificity of the antibody.

In certain embodiments, in the context of the further aspect, the ADC, preferably, the linker of the invention comprises a toxin as a third payload wherein preferably the toxin may be an auristatin. As used herein, the term “auristatin” refers to a family of anti-mitotic agents. Auristatins are anti-mitotic agents that are tubulin polymerization-blocking agents. Auristatin derivatives are also included within the definition of the term “auristatin”. Thus, in a preferred embodiment, the third payload comprises an anti-mitotic drug, or a tubulin polymerization-blocking agent.

Examples of auristatin include, but are not limited to, synthetic analogues of auristatin E (AE), monomethyl auristatin E (MMAE), monomethyl auristatin F (MMAF) and dolastatin.

In a particular preferred embodiment, the present invention relates to an ADC as defined herein, wherein said ADC, more specifically, said linker further comprises as a third payload an MMAE (Monomethyl auristatin E).

In certain embodiments, in the context of the further aspect, the ADC, preferably, the linker of the invention comprises a toxin as a third payload wherein preferably the toxin may be a maytansinoid. In the context of the present invention, the term “maytansinoid” refers to a class of highly cytotoxic drugs originally isolated from the African shrub Maytenus ovatus and further maytansinol (Maytansinol) and C-3 ester of natural maytansinol (U.S. Pat. No. 4,151,042); C-3 ester analog of synthetic maytansinol (Kupchan et al., J. Med. Chem. 21: 31-37, 1978; Higashide et al., Nature 270: 721-722, 1977; Kawai et al., Chem. Farm. Bull. 32: 3441-3451; and U.S. Pat. No. 5,416,064); C-3 esters of simple carboxylic acids (U.S. Pat. Nos. 4,248,870; 4,265,814; 4,308,268; 4,308,269; 4,309,428; 4,317,821; 4,322,348; and 4,331,598); and C-3 esters with derivatives of N-methyl-L-alanine (U.S. Pat. Nos. 4,137,230; 4,260,608; and Kawai et al., Chem. Pharm Bull. 12: 3441, 1984). Exemplary maytansinoids that may be used in the method of the invention or that may be comprised in the antibody-payload conjugate of the invention are maytansine, DM1, DM3, DM4 and/or DM21.

In certain embodiments, in the context of the further aspect, the ADC, preferably, the linker of the invention comprises a toxin as a third payload wherein preferably the toxin may be a duocarmycin. Suitable duocarmycins may be e.g. duocarmycin A, duocarmycin BI, duocarmycin B2, duocarmycin Cl, duocarmycin C2, duocarmycin D, duocarmycin SA, duocarmycin MA, and CC-1065. The term “duocarmycin” should be understood as referring also to synthetic analogs of duocarmycins, such as adozelesin, bizelesin, carzelesin, KW-2189 and CBI-TMI.

In certain embodiments, in the context of the further aspect, the ADC, preferably, the linker of the invention comprises a toxin as a third payload wherein preferably the toxin may be a NAMPT inhibitor. As used herein, the terms “NAMPT inhibitor” and “nicotinamide phosphoribosyl transferase inhibitor” refer to an inhibitor that reduces the activity of NAMPT. The term “NAMPT inhibitor” may also include prodrugs of a NAMPT inhibitor. Examples of NAMPT inhibitors include, without limitation, FK866 (also referred to as AP0866), GPP 78 hydrochloride, ST 118804, STF31, pyridyl cyanoguanidine (also referred to as C_{H}-828), GMX-1778, and P7C3. Additional NAMPT inhibitors are known in the art and may be suitable for use in the compositions and methods described herein. See, e.g., PCT Publication WO 2015/054060, U.S. Pat. Nos. 8,211,912, and 9,676,721, which are incorporated by reference herein in their entireties. In some embodiments, the NAMPT inhibitor is FK866. In some embodiments, the NAMPT inhibitor is GMX-1778.

In certain embodiments, in the context of the further aspect, the ADC, preferably, the linker of the invention comprises a toxin as a third payload wherein preferably the toxin may be a tubulysin. Tubulysins are cytotoxic peptides, which include 9 members (A-I). Tubulysin A has potential application as an anticancer agent. It arrests cells in the G2/M phase. Tubulysin A inhibits polymerization more efficiently than vinblastine and induces depolymerization of isolated microtubules. Tubulysin A has potent cytostatic effects on various tumor cell lines with IC50 in the picomolar range. Other tubulysins that may be used in the method of the invention may be tubulysin E.

In certain embodiments, in the context of the further aspect, the ADC, preferably, the linker of the invention comprises a toxin as a third payload wherein preferably the toxin may be an enediyne. The term “enediyne,” as used herein, refers to a class of bacterial natural products characterized by either nine- and ten-membered rings containing two triple bonds separated by a double bond (see, e.g., K. C. Nicolaou; A. L. Smith; E. W. Yue (1993). “Chemistry and biology of natural and designed enediynes”. PNAS 90 (13): 5881-5888; the entire contents of which are incorporated herein by reference). Some enediynes are capable of undergoing Bergman cyclization, and the resulting diradical, a 1,4-dehydrobenzene derivative, is capable of abstracting hydrogen atoms from the sugar backbone of DNA which results in DNA strand cleavage (see, e.g., S. Walker; R. Landovitz; W. D. Ding; G. A. Ellestad; D. Kahne (1992). “Cleavage behavior of calicheamicin gamma 1 and calicheamicin T”. Proc Natl Acad Sci U.S.A. 89 (10): 4608-12; the entire contents of which are incorporated herein by reference). Their reactivity with DNA confers an antibiotic character to many enediynes, and some enediynes are clinically investigated as anticancer antibiotics. Nonlimiting examples of enediynes are dynemicin, neocarzinostatin, calicheamicin, esperamicin (see, e.g., Adrian L. Smith and K. C. Bicolaou, “The Enediyne Antibiotics” J. Med. Chem., 1996, 39 (11), pp 2103-2117; and Donald Borders, “Enediyne antibiotics as antitumor agents,” Informa Healthcare; 1st edition (Nov. 23, 1994, ISBN-10: 0824789385; the entire contents of which are incorporated herein by reference). In a particular embodiment, the toxin may be calicheamicin.

In certain embodiments, in the context of the further aspect, the ADC, preferably, the linker of the invention comprises a toxin as a third payload wherein preferably the toxin may be a doxorubicin. “Doxorubicin” as used herein refers to members of the family of Anthracyclines derived from Streptomyces bacterium Streptomyces peucetius var. caesius, and includes doxorubicin, daunorubicin, epirubicin and idarubicin.

In certain embodiments, in the context of the further aspect, the ADC, preferably, the linker of the invention comprises a toxin as a third payload wherein preferably the toxin may be a kinesin spindle protein inhibitor. The term “kinesin spindle protein inhibitor” refers to a compound that inhibits the kinesin spindle protein, which involves in the assembly of the bipolar spindle during cell division. Kinesin spindle protein inhibitors are being investigated for the treatment of cancer. Examples of kinesin spindle protein inhibitor include ispinesib. Further, the term “kinesin spindle protein inhibitor” includes SB715992 or SB743921 from GlaxoSmithKline and pentamidine/chlorpromarine from CombinatoRx.

In certain embodiments, in the context of the further aspect, the ADC, preferably, the linker of the invention comprises a toxin as a third payload wherein preferably the toxin may a cryptophycin, or derivative, as described in US20180078656A1, US 20210163458 A1, US20210228726A1, which are incorporated by reference.

In certain embodiments, in the context of the further aspect, the ADC, preferably, the linker of the invention comprises a toxin as a third payload wherein preferably the toxin may be sandramycin. Sandramycin is a depsipeptide that has first been isolated from Nocardioides sp. (ATCC 39419) and has been shown to have cytotoxic and anti-tumor activity.

In certain embodiments, in the context of the further aspect, the ADC, preferably, the linker of the invention comprises a toxin as a third payload wherein preferably the toxin may be an antimetabolite or metabolite inhibitor. Antimetabolites and metabolite inhibitors are known in the art. As an example, the toxin may be pemetrexed. Pemetrexed is a chemotherapeutically active agent that falls in the class of chemotherapy drugs called folate antimetabolites. It is known to inhibit three enzymes used in purine and pyrimidine synthesis: thymidylate synthase (TS), dihydrofolate reductase (DHFR), and glycinamide ribonucleotide formyltransferase (GARFT). By inhibiting the formation of precursor purine and pyrimidine nucleotides, pemetrexed prevents the formation of DNA and RNA, which are required for the growth and survival of both normal cells and cancer cells.

In certain other embodiments, any other antimetabolite/metabolite inhibitor may be used that inhibits thymidylate synthase (TS), dihydrofolate reductase (DHFR), and/or glycinamide ribonucleotide formyltransferase (GARFT).

In certain other embodiments, said antimetabolite/metabolite inhibitor is a thymidine synthase (or thymidylate synthase) inhibitor. Thymidylate synthase inhibitors are chemical agents which inhibit the enzyme thymidylate synthase and have potential as an anticancer chemotherapy. This inhibition prevents the methylation of C5 of deoxyuridine monophosphate (dUMP) thereby inhibiting the synthesis of deoxythymidine monophosphate (dTMP). The downstream effect is promotion of cell death because cells would not be able to properly undergo DNA synthesis if they are lacking dTMP, a necessary precursor to dTTP.

Within the present invention, the thymidylate synthase inhibitor may be, without limitation, raltitrexed, pemetrexed, nolatrexed, ZD9331, GS7904L, fluorourcail, BGC-945 and OSI-7904L.

In certain embodiments, in the context of the further aspect, the ADC, preferably, the linker of the invention comprises a toxin as a third payload wherein preferably the toxin may be an amatoxin. Amatoxins (including alpha-amanitin, beta-amanitin and amanitin) are cyclic peptides composed of 8 amino acids. They can be isolated from Amanita phalloides mushrooms or prepared from the building blocks by synthesis. Amatoxins inhibit specifically the DNA-dependent RNA polymerase II of mammalian cells, and by this transcription and protein biosynthesis of the cells affected. Inhibition of transcription in a cell causes stop of growth and proliferation. Though not covalently bound, the complex between amanitin and RNA-polymerase II is very tight (KD=3 nM). Dissociation of amanitin from the enzyme is a very slow process what makes recovery of an affected cell unlikely. When in a cell the inhibition of transcription will last too long, the cell undergoes programmed cell death (apoptosis). In one preferred embodiment, term “Amatoxin” as used herein refers to an alpha-amanitin or variant thereof as described e.g. in WO2010/115630, WO2010/115629, WO2012/119787, WO2012/041504, and WO2014/135282.

In certain embodiments, in the context of the further aspect, the ADC, preferably, the linker of the invention comprises a as a third payload a DNA damage repair inhibitor. DNA repair inhibitors are known in the art. Any DNA repair inhibitor known in the art may be used as a third payload. Without being bound by theory, some examples of DNA repair inhibotors are PARP inhibitors, ATR/ATM inhibitors and Chk1 inhibitors. Further, as a DNA repair inhibitor, a kinase inhibitor may be used as a third payload. In a preferred embodiment, the kinase inhibitor is a tyrosine kinase inhibitor.

In certain embodiments, in the context of the further aspect, the ADC, preferably, the linker of the invention comprises a as a third payload a toxin, wherein preferably the toxin is a topoisomerase II inhibitor. Topoisomerase II inhibitors are known in the art. In preferred embodiments, the topoisomerase II inhibitor that may be used as a third payload may be selected from the group consisting of aminocoumarin (novobiocin), fluoro-quinolones (cinonaxin), derivative of epipodophyllotoxin (etoposide), acridine or anthraquinone derivatives (amsacrine, pixantrone).

In certain embodiments, in the context of the further aspect, the ADC, preferably, the linker of the invention consists of or comprises one or more third payload(s).

In embodiments wherein the ADC, preferably, the linker of the present invention consists or comprises more than one third payloads, said more than one third payloads are identical in structure. In preferred embodiments, said more than one third payloads which are identical in structure is one as defined herein-above.

In another preferred embodiment, said more than one third payloads are different in structure. In preferred embodiments, said more than one payloads which are different in structure is one or more of the third payloads as defined herein-above.

While the number of third payloads is not limited, in preferred embodiments, in the context of the further aspect, the linker of the invention consists of or comprises of 1, 2, 3, 4, 5, 6, 7, 8, 9 or 10 third payload(s). In a particular preferred embodiment, the linker of the present invention consists of 1 third payload.

In a preferred embodiment, the present invention relates to an ADC as defined herein, wherein the third payload is linked to the N- or C-terminus of the peptide linker or to a side-chain of an amino acid residue comprised in the peptide linker; preferably wherein the third payload is linked to the C-terminus of the peptide linker.

In a preferred embodiment, the present invention relates to an ADC as defined herein, wherein the first payload and/or second payload and/or the third payload are linked to the N- or C-terminus of the peptide linker or to a side-chain of an amino acid residue comprised in the peptide linker; preferably
wherein the first payload and the second payload and the third payload are linked to the N-terminus of the peptide linker; or
wherein the first payload and the second payload and the third payload are linked to the C-terminus of the peptide linker;
wherein the first payload and the second payload are linked to the N-terminus of the peptide linker and wherein the third payload is linked to the C-terminus of the peptide linker; or
wherein the first payload and the third payload are linked to the N-terminus of the peptide linker and wherein the second payload is linked to the C-terminus of the peptide linker; or
wherein the second payload and the third payload are linked to the N-terminus of the peptide linker and wherein the first payload is linked to the C-terminus of the peptide linker; or
wherein the first payload is linked to the N-terminus of the peptide linker and wherein the second payload and the third payload are linked to the C-terminus of the peptide linker; or
wherein the second payload is linked to the N-terminus of the peptide linker and wherein the first payload and the third payload are linked to the C-terminus of the peptide linker; or
wherein the third payload is linked to the N-terminus of the peptide linker and wherein the first payload and the second payload are linked to the C-terminus of the peptide linker.

In a preferred embodiment, the present invention relates to an ADC as defined herein, wherein the linker consists or comprises the following structure:

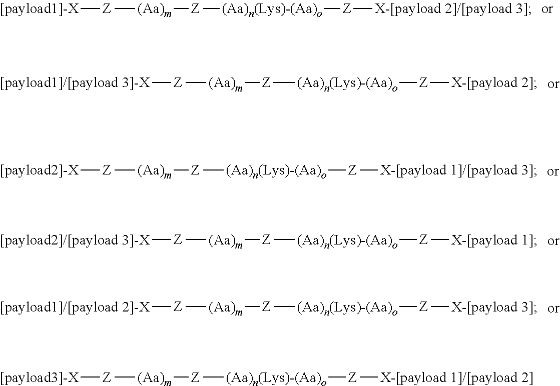

wherein
[payload 1] is said first payload;
[payload 2] is said second payload;
[payload 3] is said third payload;
(Aa) is any amino acid residue;
m, n and o may be integers ranging from 0 to 10, preferably 0 to 6, more preferably 0 to 4;
(Lys) is a lysine residue, a lysine mimetic or a lysine derivative;
Z is either absent or a spacer comprising an alkyl or a heteroalkyl group, preferably wherein the spacer is (C_{H}2)₂; and
X is either absent or a self-immolative group, preferably PABC.

In a particular embodiment, the invention relates to the ADC according to the invention, wherein two payloads are linked to the same functional group of the peptide linker, preferably via a chemical linker comprising a disubstituted amine.

That is, in a preferred embodiment, the invention relates to the ADC according to the invention, wherein the linker consists of or comprises the structure:

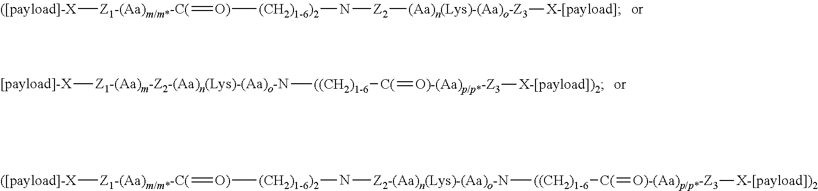

wherein
[payload] is each independently a payload selected from the first, second and third payload, wherein the linker comprises all three payloads;
(Aa) is any amino acid residue;
m, m*, n, o, p and p* may be integers ranging from 0 to 10, preferably 0 to 6, more preferably 0 to 4, even more preferably wherein n+o is >0;
(Lys) is a lysine residue, a lysine mimetic or a lysine derivative;
Z₁₋₃ is either absent or a spacer comprising an alkyl or a heteroalkyl group, preferably wherein the spacer comprises (CH₂)₂, even more preferably wherein Z₂ is a dicarboxylic acid linker; and
X is either absent or a self-immolative group, preferably PABC.

In an even more preferred embodiment, the invention relates to the ADC according to the invention, wherein the linker consists of or comprises the structure:

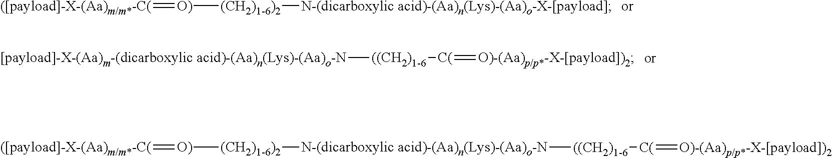

wherein
[payload] is each independently a payload selected from the first, second and third payload, wherein the linker comprises all three payloads;
(Aa) is any amino acid residue;
m, m*, p and p* are integers ranging from 1 to 10, preferably 1 to 6, more preferably 1 to 4;
n and o may be integers ranging from 0 to 10, preferably 0 to 6, more preferably 0 to 4, wherein n+o is >0;
(Lys) is a lysine residue, a lysine mimetic or a lysine derivative;
(dicarboxylic acid) is dicarboxylic acid as defined herein linking the N-terminal end of (Aa)ₘ or the disubstituted amine (N) to the N-terminal end of (Aa)ₙ or (Lys); and
X is either absent or a self-immolative group, preferably PABC.

The linkers preferably comprise at least one additional positively charged amino acid residue in addition to the lysine residue, as explained in detail elsewhere herein. That is, in a particular embodiment, the invention relates to the ADC according to the invention, wherein (Aa)ₙ-(Lys)-(Aa)ₒ comprises the sequence motif Arg-Lys (RK) or His-Lys (HK) (in N->C direction). In another particular embodiment, the invention relates to the ADC according to the invention, wherein (Aa)ₙ-(Lys)-(Aa)ₒ is or comprises RK or RKAA (in N->C direction).

In a preferred embodiment, the present invention relates to an ADC as defined herein, wherein said ADC comprises more than one first payloads and/or more than one second payloads and/or more than one third payloads.

As already outlined above, the peptide linker in accordance with the present invention comprises or contains at least two payloads, i.e., the “first payload” and the “second payload”.

In the embodiments wherein a “third payload” is comprised, the peptide linker in accordance with the present invention comprises or contains at least three payloads.

If the peptide linker comprises or contains only three payloads, these three payloads are different in structure, i.e., the first payload is a camptothecin cytotoxic molecule which is cell-permeable; and the second payload is a camptothecin cytotoxic molecule which is not cell-permeable; and the third payload is not a camptothecin derivative cytotoxic molecule and is preferably a toxin, preferably, anti-mitotic drug, tubulin polymerization-blocking agent, and/or auristatin, more preferably, an MMAE as defined herein.

If the peptide linker comprises or contains more than three payloads, the payloads may be identical or may be different in structure while at least two of these payloads may be identical in structure.

Coupling two or more identical payloads to a peptide linker allows increasing the concentration of the payload in the target tissue or cell of an antibody-payload conjugate. For example, if the peptide linker of an antibody-payload conjugate comprises two or more identical toxins (resulting in a DAR>4 ADC), the concentration of the toxin in the target tissue or cell will be higher compared to a conventional DAR2 ADC. With the peptide linkers of the present invention, ADCs comprising 4, 6 or 8 identical payload molecules may be obtained.

In the embodiments wherein a third payload is comprised, in a particular embodiment, the peptide linker in accordance with the present invention comprises between 3 and 6 payloads. In a particular embodiment, the peptide linker in accordance with the present invention comprises or contains 3 payloads, i.e., a as a first payload a single topoisomerase I inhibitor which is cell-permeable; and as a second payload a single topoisomerase I inhibitor which is not cell-permeable; and as a third payload a single toxin, preferably, anti-mitotic drug, tubulin polymerization-blocking agent, and/or auristatin, more preferably, an MMAE as defined herein.

Further embodiments are preferred that correspond to the above-described embodiments wherein the peptide linker in accordance with the present invention comprises between 2 and 4 payloads, yet, supplemented with one or two or three third payloads.

In the embodiments wherein a third payload is comprised, these above-defined three payloads can be coupled in different ways to a peptide linker.

As mentioned above, the inventors identified different ways to couple two or more payloads to a peptide linker.

In certain embodiments wherein a third payload is comprised, the first payload and/or second payload and/or the third payload are linked to the N- or C-terminus of the peptide linker or to a side-chain of an amino acid residue comprised in the peptide linker.

In certain embodiments wherein a third payload is comprised, two or three payloads may be coupled to the C-terminal end of a peptide linker. In other embodiments two or three payloads may be coupled to the N-terminal end of a peptide linker. In yet another embodiment, one or two or three payloads may be coupled each to the N-terminal end of a peptide linker and to the C-terminal end of a peptide linker.

In certain embodiments wherein a third payload is comprised, three payloads may be coupled to the C-terminal end of a peptide linker. In other embodiments three payloads may be coupled to the N-terminal end of a peptide linker. In yet another embodiment, one or two or three payloads may be coupled each to the N-terminal end of a peptide linker and to the C-terminal end of a peptide linker.

In another particular embodiment wherein a third payload is comprised, one or more payload is attached to the N-terminal end of an amine-comprising peptide linker and wherein one or more payload is attached to the C-terminal end of said amine-comprising peptide linker.

In another particular embodiment wherein a third payload is comprised, two payloads are attached to the N-terminal end of a peptide linker and one payload is attached to the C-terminal end of said peptide linker. An example for a corresponding arrangement is illustrated in FIG. 8.

In certain embodiments, one or two or three payloads may be attached each to the N-terminal end of an amine-comprising peptide linker and one or two or three payloads may be attached each to the C-terminal end of said amine-comprising peptide linker.

In a preferred embodiment, the present invention relates to an ADC as defined herein, wherein the third payload is an MMAE.

In a preferred embodiment, the present invention relates to an ADC as defined herein, wherein the linker comprises or consists of the following structure:

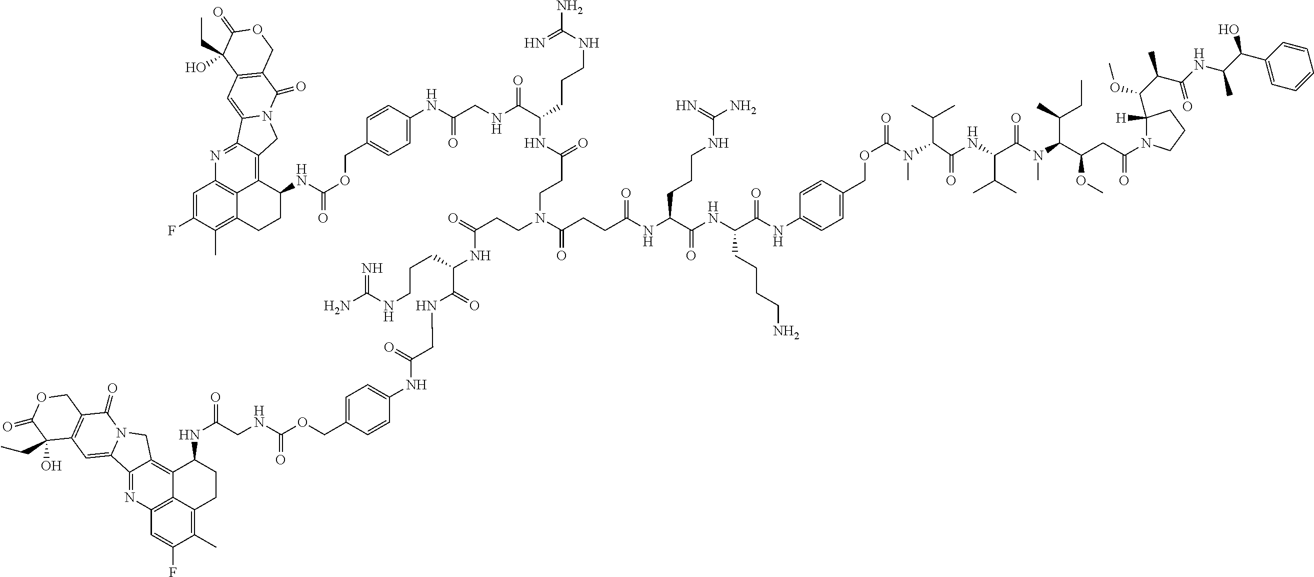 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](C(C)C)N(C)C(=O)OCc1ccc(NC(=O)[C@H](CCCCN)NC(=O)[C@H](CCCNC(=N)N)NC(=O)CCC(=O)N(CCC(=O)N[C@@H](CCCNC(=N)N)C(=O)NCC(=O)Nc2ccc(COC(=O)NCC(=O)N[C@H]3CCc4c(C)c(F)cc5nc6c(c3c45)Cn3c-6cc4c(c3=O)COC(=O)[C@]4(O)CC)cc2)CCC(=O)N[C@@H](CCCNC(=N)N)C(=O)NCC(=O)Nc2ccc(COC(=O)N[C@H]3CCc4c(C)c(F)cc5nc6c(c3c45)Cn3c-6cc4c(c3=O)COC(=O)[C@]4(O)CC)cc2)cc1)C(C)C]

In a preferred embodiment, the present invention relates to an ADC as defined herein, wherein said ADC consists of two first payloads and two second payloads and two third payloads (drug-to-antibody ratio of 6 “DAR6”).

Accordingly, in this embodiment, the peptide linker in accordance with the present invention comprises or contains 3 payloads, i.e.,
as a first payload: one topoisomerase I inhibitor which is cell-permeable; and as a second payload: one topoisomerase I inhibitor which is not cell-permeable; and
one third payload: preferably, as a third payload one toxin, preferably, anti-mitotic drug, tubulin polymerization-blocking agent, and/or auristatin, more preferably, MMAE as defined herein.

In a particular preferred embodiment, the present invention relates to an ADC as defined herein having the formula A-L, wherein A is an antibody or an antibody fragment and wherein L is a linker, said linker comprising:
as a first payload a camptothecin cytotoxic molecule which is cell-permeable; and
as a second payload a camptothecin cytotoxic molecule which is not cell-permeable;
wherein the cytotoxic molecule of the first and second payload is an exatecan;
wherein the second payload has a glycine residue linked to said camptothecin cytotoxic molecule of the second payload;
wherein said ADC consists of two first payloads and two second payloads (drug-to-antibody ratio of 4 “DAR4”); and
wherein said antibody is and IgG, preferably, an IgG1 antibody.

As regards the definitions of this ADC as well as preferred embodiments thereof, same applies, mutatis mutandis, as has been set forth above in the context of the ADC of the present invention.

In a particular preferred embodiment, the present invention relates to an ADC as defined herein having the formula A-L, wherein A is an antibody or an antibody fragment and wherein L is a linker, said linker comprising:
as a first payload a camptothecin cytotoxic molecule which is cell-permeable; and
as a second payload a camptothecin cytotoxic molecule which is not cell-permeable; and
a third payload as defined above, preferably, a toxin, more preferably, anti-mitotic drug, tubulin polymerization-blocking agent, and/or auristatin, even more preferably, MMAE, wherein the cytotoxic molecule of the first and second payload is an exatecan;
wherein the second payload has a glycine residue linked to said camptothecin cytotoxic molecule of the second payload;
wherein said ADC consists of two first payloads and two second payloads and two third payloads (drug-to-antibody ratio of 6 “DAR6”); and
wherein said antibody is and IgG, preferably, an IgG1 antibody.

As regards the definitions of this ADC as well as preferred embodiments thereof, same applies, mutatis mutandis, as has been set forth above in the context of the ADC of the present invention.

In a particular preferred embodiment, the present invention relates to an ADC as defined herein having the formula A-L, wherein A is an antibody or an antibody fragment and wherein L is a linker, said linker comprising:
as a first payload a camptothecin cytotoxic molecule which is cell-permeable; and
as a second payload a camptothecin cytotoxic molecule which is not cell-permeable;
wherein the cytotoxic molecule of the first and second payload is an exatecan;
wherein the second payload has a glycine residue linked to said camptothecin cytotoxic molecule of the second payload;
wherein said ADC consists of two first payloads and two second payloads (drug-to-antibody ratio of 4 “DAR4”); and
wherein said antibody is Trastuzumab.

As regards the definitions of this ADC as well as preferred embodiments thereof, same applies, mutatis mutandis, as has been set forth above in the context of the ADC of the present invention.

In a particular preferred embodiment, the present invention relates to an ADC as defined herein having the formula A-L, wherein A is an antibody or an antibody fragment and wherein L is a linker, said linker comprising:
as a first payload a camptothecin cytotoxic molecule which is cell-permeable; and
as a second payload a camptothecin cytotoxic molecule which is not cell-permeable;
wherein the cytotoxic molecule of the first and second payload is an exatecan;
wherein the second payload has a glycine residue linked to said camptothecin cytotoxic molecule of the second payload;
wherein said ADC consists of two first payloads and two second payloads (drug-to-antibody ratio of 4 “DAR4”); and
wherein said antibody is an antibody that specifically binds to the antigen NaPi2b.

In the context of this particularly preferred embodiment, in an even more preferred embodiment, the linker comprises or consists of the following structure:

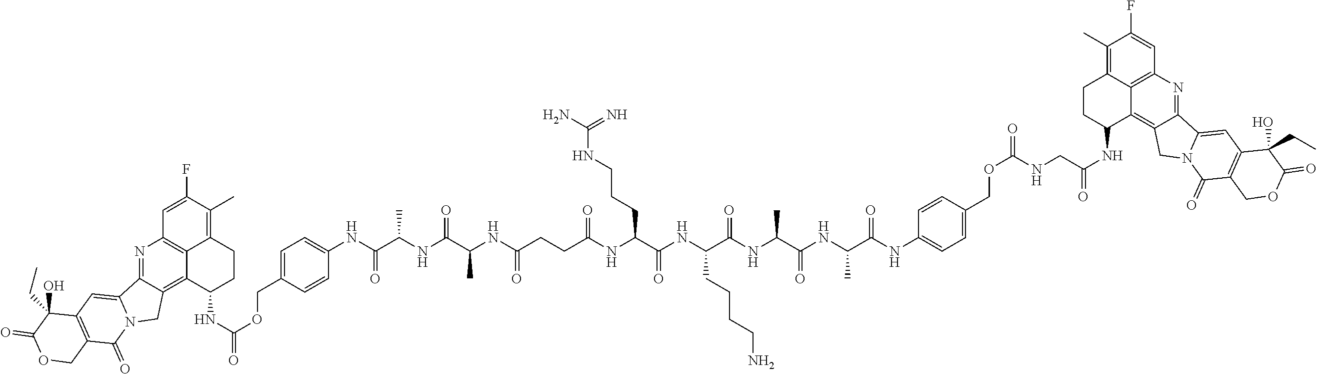

In a particular preferred embodiment, the present invention relates to an ADC comprising an antibody that specifically binds to the antigen NaPi2b,
preferably wherein the antibody comprises a heavy chain CDR1 (CDR-H1, Kabat) as set forth in SEQ ID NO:136, a heavy chain CDR2 (CDR-H2, Kabat) as set forth in SEQ ID NO:137, a heavy chain CDR3 (CDR-H3, Kabat) as set forth in SEQ ID NO:138, a light chain CDR1 (CDR-L1, Kabat) as set forth in SEQ ID NO:139, a light chain CDR2 (CDR-L2, Kabat) as set forth in SEQ ID NO:140, and a light chain CDR3 (CDR-L3, Kabat) as set forth in SEQ ID NO:141,
more preferably wherein the antibody comprises a heavy chain variable region as set forth in SEQ ID NO:134 and a light chain variable region as set forth in SEQ ID NO:135,
even more preferably wherein the antibody comprises a heavy chain as set forth in SEQ ID NO:98 and a light chain as set forth in SEQ ID NO:99 or a heavy chain as set forth in SEQ ID NO:100 and a light chain as set forth in SEQ ID NO:101;
and a linker having the structure:

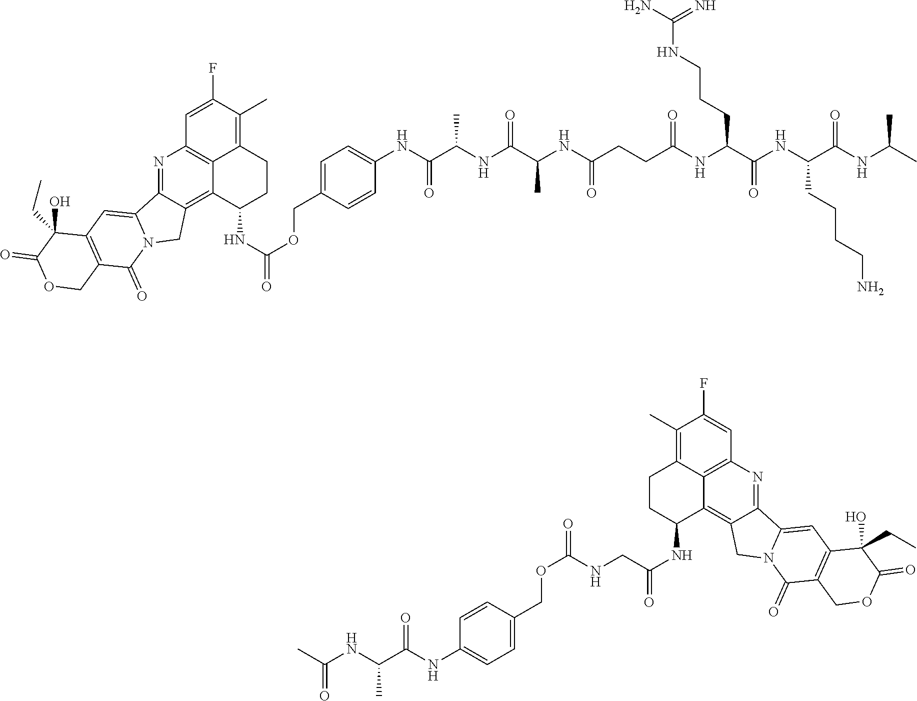

wherein the linker is conjugated via the lysine residue to glutamine residue Q295 (EU numbering) of the heavy chain of the antibody.

As regards the definitions of this ADC as well as preferred embodiments thereof, same applies, mutatis mutandis, as has been set forth above in the context of the ADC of the present invention.

In a particular preferred embodiment, the present invention relates to an ADC as defined herein having the formula A-L, wherein A is an antibody or an antibody fragment and wherein L is a linker, said linker comprising:
as a first payload a camptothecin cytotoxic molecule which is cell-permeable; and
as a second payload a camptothecin cytotoxic molecule which is not cell-permeable;
wherein the cytotoxic molecule of the first and second payload is an exatecan;
wherein the second payload has a glycine residue linked to said camptothecin cytotoxic molecule of the second payload;
wherein said ADC consists of two first payloads and two second payloads (drug-to-antibody ratio of 4 “DAR4”); and
wherein said antibody is an antibody that specifically binds to the antigen Nectin-4.

As regards the definitions of this ADC as well as preferred embodiments thereof, same applies, mutatis mutandis, as has been set forth above in the context of the ADC of the present invention.

In a particular preferred embodiment, the present invention relates to an ADC as defined herein having the formula A-L, wherein A is an antibody or an antibody fragment and wherein L is a linker, said linker comprising:
as a first payload a camptothecin cytotoxic molecule which is cell-permeable; and
as a second payload a camptothecin cytotoxic molecule which is not cell-permeable; and a third payload as defined above, preferably, a toxin, more preferably, anti-mitotic drug, tubulin polymerization-blocking agent, and/or auristatin, even more preferably, MMAE, wherein the cytotoxic molecule of the first and second payload is an exatecan;
wherein the second payload has a glycine residue linked to said camptothecin cytotoxic molecule of the second payload;
wherein said ADC consists of two first payloads and two second payloads and two third payloads (drug-to-antibody ratio of 6 “DAR6”); and
wherein said antibody is Trastuzumab.

As regards the definitions of this ADC as well as preferred embodiments thereof, same applies, mutatis mutandis, as has been set forth above in the context of the ADC of the present invention.

In a particular preferred embodiment, the present invention relates to an ADC as defined herein having the formula A-L, wherein A is an antibody or an antibody fragment and wherein L is a linker, said linker comprising:
as a first payload a camptothecin cytotoxic molecule which is cell-permeable; and
as a second payload a camptothecin cytotoxic molecule which is not cell-permeable; and
a third payload as defined above, preferably, a toxin, more preferably, anti-mitotic drug, tubulin polymerization-blocking agent, and/or auristatin, even more preferably, MMAE, wherein the cytotoxic molecule of the first and second payload is an exatecan;
wherein the second payload has a glycine residue linked to said camptothecin cytotoxic molecule of the second payload;
wherein said ADC consists of two first payloads and two second payloads and two third payloads (drug-to-antibody ratio of 6 “DAR6”); and
wherein said antibody is an antibody that specifically binds to the antigen NaPi2b.

As regards the definitions of this ADC as well as preferred embodiments thereof, same applies, mutatis mutandis, as has been set forth above in the context of the ADC of the present invention.

In a particular preferred embodiment, the present invention relates to an ADC as defined herein having the formula A-L, wherein A is an antibody or an antibody fragment and wherein L is a linker, said linker comprising:
as a first payload a camptothecin cytotoxic molecule which is cell-permeable; and
as a second payload a camptothecin cytotoxic molecule which is not cell-permeable; and a third payload as defined above, preferably, a toxin, more preferably, anti-mitotic drug, tubulin polymerization-blocking agent, and/or auristatin, even more preferably, MMAE, wherein the cytotoxic molecule of the first and second payload is an exatecan;
wherein the second payload has a glycine residue linked to said camptothecin cytotoxic molecule of the second payload;
wherein said ADC consists of two first payloads and two second payloads and two third payloads (drug-to-antibody ratio of 6 “DAR6”); and
wherein said antibody is an antibody that specifically binds to the antigen Nectin-4, preferably wherein the antibody is m290 as defined herein.

In the context of this particularly preferred embodiment, in an even more preferred embodiment, the linker comprises or consists of the following structure:

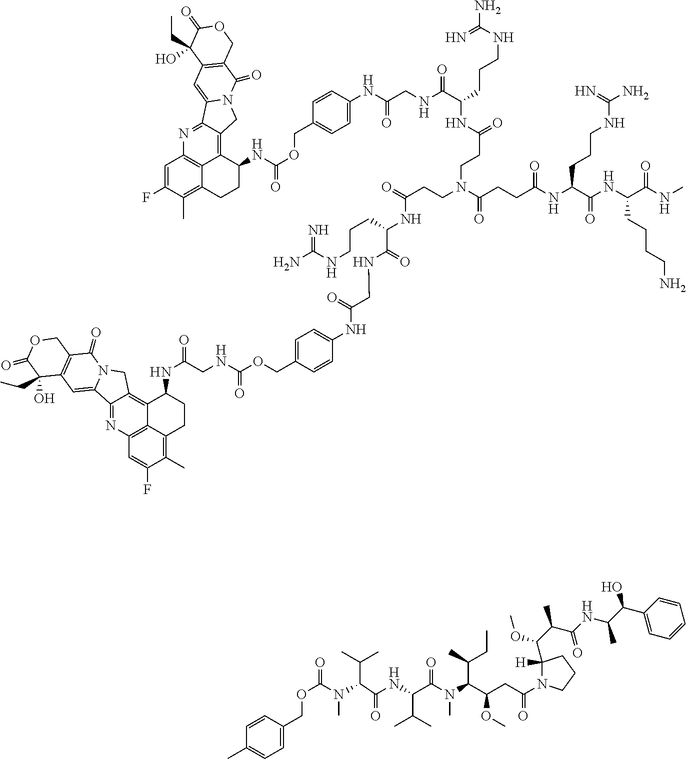

In a particular preferred embodiment, the present invention relates to an ADC comprising an antibody that specifically binds to the antigen Nectin-4,
preferably wherein the antibody comprises a heavy chain CDR1 (CDR-H1, Kabat) as set forth in SEQ ID NO:128, a heavy chain CDR2 (CDR-H2, Kabat) as set forth in SEQ ID NO:129, a heavy chain CDR3 (CDR-H3, Kabat) as set forth in SEQ ID NO:130, a light chain CDR1 (CDR-L1, Kabat) as set forth in SEQ ID NO:131, a light chain CDR2 (CDR-L2, Kabat) as set forth in SEQ ID NO:132, and a light chain CDR3 (CDR-L3, Kabat) as set forth in SEQ ID NO:133,
more preferably wherein the antibody comprises a heavy chain variable region as set forth in SEQ ID NO:126 and a light chain variable region as set forth in SEQ ID NO:127,
even more preferably wherein the antibody comprises a heavy chain as set forth in SEQ ID NO:96 and a light chain as set forth in SEQ ID NO:97;
and a linker having the structure:

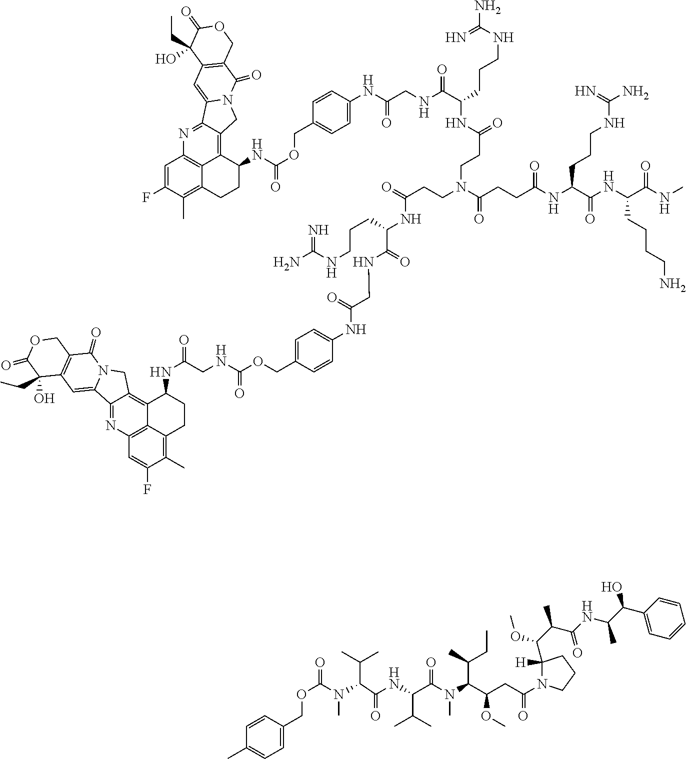

wherein the linker is conjugated via the lysine residue to glutamine residue Q295 (EU numbering) of the heavy chain of the antibody.

As regards the definitions of this ADC as well as preferred embodiments thereof, same applies, mutatis mutandis, as has been set forth above in the context of the ADC of the present invention.

The ADC of the present invention is particularly useful in medical settings.

In a preferred embodiment, the present invention relates to a pharmaceutical composition comprising the ADC as defined herein and at least one pharmaceutically acceptable ingredient.

The term “pharmaceutical composition” as used herein refers to any composition comprising a chemical substance or active ingredient which composition is intended for use in the medical cure, treatment, or prevention of disease and which is in such a form as to permit the active ingredient to be effective. In particular, a pharmaceutical composition does not contain excipients which are unacceptably toxic to a subject to which the composition is to be administered. The pharmaceutical compositions are sterile, i.e., aseptic and free from all living microorganisms and their spores. The pharmaceutical composition of the present invention is preferably liquid.

The pharmaceutical composition according to the invention comprises an antibody-drug conjugate as disclosed herein. Pharmaceutical compositions comprising an antibody-drug conjugate are preferably used for the treatment of diseases.

The pharmaceutical composition according to the invention may comprise at least one pharmaceutically acceptable ingredient.

A pharmaceutically acceptable ingredient refers to an ingredient in a pharmaceutical formulation, other than an active ingredient, which is nontoxic to a subject. A pharmaceutically acceptable ingredient includes, but is not limited to, a buffer, excipient, stabilizer, or preservative.

Pharmaceutical formulations of the antibody-payload conjugates described herein are prepared by mixing such conjugates having the desired degree of purity with one or more optional pharmaceutically acceptable ingredients (Flemington's Pharmaceutical Sciences 16th edition, Osol, A. Ed. (1980)), in the form of lyophilized formulations or aqueous solutions. Pharmaceutically acceptable ingredients are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn protein complexes); and/or non-ionic surfactants such as polyethylene glycol (PEG). Exemplary pharmaceutically acceptable ingredients herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. For example, a sHASEGP may be combined with one or more additional glycosaminoglycanases such as chondroitinases.

In a particular embodiment, the invention relates to a pharmaceutical composition according to the invention comprising at least one additional therapeutically active agent.

The pharmaceutical composition comprising the antibody-payload conjugate may comprise one or more additional therapeutically active agents. It is to be understood that the antibody-payload conjugates may be used in various therapeutic areas. As such, the additional therapeutically active agent in the pharmaceutical composition may vary depending on the use of the pharmaceutical composition.

In certain embodiments, as outlined in more detail further below, a pharmaceutical composition comprising an antibody-payload conjugate according to the invention may be used in the treatment of a neoplactic disorder, preferably in the treatment of cancer. In such embodiment, the pharmaceutical composition may comprise one or more additional anti-cancer drugs. The term “anti-cancer” drug is used herein to refer to one or a combination of drugs conventionally used to treat cancer.

For example, a pharmaceutical composition comprising an antibody-payload conjugate according to the invention may further comprise one or more chemotherapeutic agents. As used herein, the term “chemotherapeutic agent” or “chemotherapy agent” or “chemotherapeutic drug” refer to an agent that reduces, prevents, mitigates, limits, and/or delays the growth of metastases or neoplasms, or kills neoplastic cells directly by necrosis or apoptosis of neoplasms or any other mechanism, or that can be otherwise used, in a pharmaceutically-effective amount, to reduce, prevent, mitigate, limit, and/or delay the growth of metastases or neoplasms in a subject with neoplastic disease. Chemotherapeutic agents include, for example, fluoropyrimidines; pyrimidine nucleosides; purine nucleosides; anti-folates, platinum agents; anthracyclines/anthracenediones; epipodophyllotoxins; camptothecins; hormones; hormonal complexes; antihormonals; enzymes, proteins, peptides and polyclonal and/or monoclonal antibodies; vinca alkaloids; taxanes; epothilones; antimicrotubule agents; alkylating agents; antimetabolites; topoisomerase inhibitors; antivirals; and various other cytotoxic and cytostatic agents.

In a particular embodiment, the invention relates to the antibody-payload conjugate according to the invention, or the pharmaceutical composition according to the invention for use in therapy.

Thus, the antibody-payload conjugate or the pharmaceutical composition according to the invention may be used in the treatment of a subject/patient. An individual or subject or patient is preferably a mammal. Mammals include, but are not limited to, domesticated animals (e.g., cows, sheep, cats, dogs, and horses), primates (e.g., humans and non-human primates such as macaques), rabbits, and rodents (e.g., mice and rats). In certain embodiments, the individual or subject is a human.

In a preferred embodiment, the present invention relates to an ADC as defined herein, for use in a method of treating a patient suffering from, being at risk of developing, and/or being diagnosed for a neoplastic disease.

A neoplastic disease or a neoplasm is commonly understood, in its broadest sense, as a type of abnormal and excessive growth of tissue. The process that occurs to form or produce a neoplasm is called neoplasia. This abnormal growth usually forms a mass, when it may be called a tumour or tumor. ICD-10 classifies neoplasms into four main groups: benign neoplasms, in situ neoplasms, malignant neoplasms, and neoplasms of uncertain or unknown behavior. Malignant neoplasms are also simply known as cancers. A neoplasm can be benign, potentially malignant, or malignant (cancer).

The patient suffering from cancer may be a patient who has not been previously treated with any anti-cancer therapy. However, the patient suffering from cancer may also be a patient who was refractory to a previous anti-cancer treatment.

The term “neoplastic disease” as used herein refers to a condition characterized by uncontrolled, abnormal growth of cells. Neoplastic diseases include cancer. Examples of cancer include but are not limited to, carcinoma, lymphoma, blastoma, sarcoma, and leukemia. More particular examples of such cancers include breast cancer, prostate cancer, colon cancer, squamous cell cancer, small-cell lung cancer, non-small cell lung cancer, ovarian cancer, cervical cancer, gastrointestinal cancer, pancreatic cancer, glioblastoma, liver cancer, bladder cancer, hepatoma, colorectal cancer, uterine cervical cancer, endometrial carcinoma, salivary gland carcinoma, kidney cancer, vulval cancer, thyroid cancer, hepatic carcinoma, skin cancer, melanoma, brain cancer, ovarian cancer, neuroblastoma, myeloma, various types of head and neck cancer, acute lymphoblastic leukemia, acute myeloid leukemia, Ewing sarcoma and peripheral neuroepithelioma. Preferred cancers include liver cancer, lymphoma, acute lymphoblastic leukemia, acute myeloid leukemia, Ewing sarcoma and peripheral neuroepithelioma.

The antibody-payload conjugates according to the invention are preferably used for the treatment of cancer. As such, in certain embodiments, the antibody-payload conjugates according to invention comprise an antibody that specifically binds to an antigen that is present on a tumor cell. In certain embodiments, the antigen may be an antigen on the surface of a tumor cell. In certain embodiments, the antigen on the surface of the tumor cell may be internalized into the cell together with the antibody-payload conjugate upon binding of the antibody-payload conjugate to the antigen.

In a preferred embodiment, the present invention relates to an ADC as defined herein or the pharmaceutical composition as defined herein,
wherein the antibody-payload conjugate comprises Trastuzumab and wherein the neoplastic disease is a HER2-positive cancer, in particular HER2-positive breast, gastric, ovarian or lung cancer;
wherein the antibody-payload conjugate comprises Polatuzumab and wherein the neoplastic disease is a B-cell associated cancer; preferably, wherein the B-cell associated cancer is non-Hodgkin lymphoma, in particular wherein the B-cell associated cancer is diffuse large B-cell lymphoma; or
wherein the antibody-payload conjugate comprises Enfortumab or an Enfortumab variant, or m290, and wherein the neoplastic disease is a Nectin-4 positive cancer, in particular Nectin-4 positive pancreatic cancer, lung cancer, bladder cancer or breast cancer; or
wherein the antibody-payload conjugate comprises Upifitamab and wherein the neoplastic disease is a NaPi2b positive cancer, in particular NaPi2b positive lung or ovarian cancer.

Thus, in certain embodiments, the antibody-payload conjugate and/or the pharmaceutical composition according to the invention may be used in the treatment of HER2-positive cancers.

That is, in a particular embodiment, the invention relates to the antibody-payload conjugate or the pharmaceutical composition for use according to the invention, wherein the antibody-payload conjugate comprises Trastuzumab and wherein the neoplastic disease is a HER2-positive cancer, in particular HER2-positive breast, gastric, ovarian or lung cancer.

For this, it is preferred that the antibody-payload conjugate comprises an anti-HER2/neu antibody as disclosed herein, preferably wherein the anti-HER2/neu antibody is internalized into a target cell upon binding to HER2/neu. In certain embodiments, the anti-HER2/neu antibody is Trastuzumab with a heavy chain as set forth in SEQ ID NO:73 and a light chain as set forth in SEQ ID NO:74.

In certain embodiments, the anti-HER2/neu antibody comprised in the antibody-payload conjugate or the pharmaceutical composition may be conjugated to any one of the linkers shown in FIGS. 4, 6, 7 or 8 or any one of the linkers disclosed herein.

A HER2-positive cancer, as used herein, may be, without limitation HER2-positive breast, gastric, ovarian or lung cancer. The skilled person is able to determine whether a cancer is a HER2-positive cancer. For example, tumor cells may be isolated in a biopsy and the presence of HER2/neu may be determined with any method known in the art.

Further, the anti-HER2/neu antibody-payload conjugate and/or the pharmaceutical composition comprising an anti-HER2/neu antibody-payload conjugate may be used in conjunction with other therapies that are suitable for the treatment of HER2-positive cancers.

Thus, in a particular embodiment, the invention relates to the antibody-payload conjugate or the pharmaceutical composition for use according to the invention, wherein the antibody-payload conjugate or the pharmaceutical composition is administered in combination with lapatinib, capecitabine and/or a taxane.

It is to be understood that the antibody-payload conjugate or the pharmaceutical composition does not necessarily have to be administered at the same time as the additional therapeutic agent, such as lapatinib, capecitabine and/or a taxane. Instead the antibody-payload conjugate or the pharmaceutical composition may be administered with a different administration schedule and, consequently, on different days as other therapeutic agents that are used for the treatment of the same disease.

In certain embodiments, the antibody-payload conjugate and/or the pharmaceutical composition according to the invention may be used in the treatment of B-cell-associated cancer.

Thus, in a particular embodiment, the invention relates to the antibody-payload conjugate or the pharmaceutical composition for use according to the invention, wherein the antibody-payload conjugate comprises Polatuzumab and wherein the neoplastic disease is a B-cell associated cancer.

For this, it is preferred that the antibody-payload conjugate comprises an anti-CD79b antibody as disclosed herein, preferably wherein the anti-CD79b antibody is internalized into a target cell upon binding to CD79b. In certain embodiments, the anti-CD79b antibody is Polatuzumab with a heavy chain as set forth in SEQ ID NO:71 and a light chain as set forth in SEQ ID NO:72.

In certain embodiments, the anti-CD79b antibody comprised in the antibody-payload conjugate or the pharmaceutical composition may be conjugated to any one of the linkers shown in FIGS. 4, 6, 7 or 8 or any one of the linkers disclosed herein.

A B-cell associated cancer may be any one selected from a group consisting of: high, intermediate and low grade lymphomas (including B cell lymphoma such as, for example, mucosa-associated lymphoid tissue B cell lymphoma and non-Hodgkin's lymphoma(NHL), mantle cell lymphoma, Burkitt's lymphoma, small lymphocytic lymphoma, marginal Zone lymphoma, diffuse large B cell lymphoma, follicular lymphoma, and Hodgkin's lymphoma and T cell lymphomas) and leukemias (including secondary leukemia, chronic lymphocytic leukemia(CLL), such as B cell leukemia (CD5+ B lymphocytes), myeloid leukemia, such as acute myeloid leukemia, chronic myeloid leukemia, lymphoid leukemia, such as acute lymphoblastic leukemia (ALL) and myelodysplasia), and other hematological and/or B cell—or T-cell—associated cancers, including cancers of additional hematopoietic cells, including polymorphonuclear leukocytes, such as basophils, eosinophils, neutrophils and monocytes, dendritic cells, platelets, erythrocytes and natural killer cells. Also included are cancerous B cell proliferative disorders selected from the following: lymphoma, non-Hodgkins lymphoma(NHL) aggressive NHL, relapsed aggressive NHL, relapsed indolent NHL, refractory NHL, refractory indolent NHL, chronic lymphocytic leukemia (CLL), Small lymphocytic lymphoma, leukemia, hairy cell leukemia(HCL), acute lymphocytic leukemia (ALL), and mantle cell lymphoma.

In a particular embodiment, the invention relates to the antibody-payload conjugate or the pharmaceutical composition for use according to the invention, wherein the B-cell associated cancer is non-Hodgkin lymphoma, in particular wherein the B-cell associated cancer is diffuse large B-cell lymphoma.

Further, the anti-CD79b antibody-payload conjugate and/or the pharmaceutical composition comprising an anti-CD79b antibody-payload conjugate may be used in conjunction with other therapies that are suitable for the treatment of B-cell-associated cancer.

Thus, in a particular embodiment the invention relates to the antibody-payload conjugate or the pharmaceutical composition for use according to the invention, wherein the antibody-payload conjugate or the pharmaceutical composition is administered in combination with bendamustine and/or rituximab.

It is to be understood that the antibody-payload conjugate or the pharmaceutical composition does not necessarily have to be administered at the same time as the additional therapeutic agent, such as bendamustine and/or rituximab. Instead the antibody-payload conjugate or the pharmaceutical composition may be administered with a different administration schedule and, consequently, on different days as other therapeutic agents that are used for the treatment of the same disease.

In certain embodiments, the antibody-payload conjugate and/or the pharmaceutical composition according to the invention may be used in the treatment of Nectin-4-positive cancers.

That is, in a particular embodiment, the invention relates to the antibody-payload conjugate or the pharmaceutical composition for use according to the invention, wherein the antibody-payload conjugate comprises Enfortumab or an Enfortumab variant, or m290, and wherein the neoplastic disease is a Nectin-4 positive cancer, in particular Nectin-4 positive pancreatic cancer, lung cancer, bladder cancer or breast cancer.

For this, it is preferred that the antibody-payload conjugate comprises an anti-Nectin-4 antibody as disclosed herein, preferably wherein the anti-Nectin-4 antibody is internalized into a target cell upon binding to Nectin-4. In certain embodiments, the anti-Nectin-4 antibody is Enfortumab with a heavy chain as set forth in SEQ ID NO:75 and a light chain as set forth in SEQ ID NO:95, 76 or 77.

Moreover, as outlined above, in a preferred embodiment, the invention relates to the antibody-payload conjugate according to the invention, wherein the antibody is an antibody, preferably, an IgG antibody comprising at Kabat position 234 an A and/or at Kabat position 235 an A.

Accordingly, in certain embodiments, the antibody is Enfortumab or a variant thereof comprising at Kabat position 234 an A and at Kabat position 235 an A with a heavy chain as set forth in SEQ ID NO:94.

In certain embodiments, the antibody is Enfortumab comprising at Kabat position 234 an A and at Kabat position 235 an A with a heavy chain as set forth in SEQ ID NO:94 and a light chain as set forth in SEQ ID NO:95.

In certain other embodiments, the present invention relates to an ADC as defined herein, wherein the antibody is m290 (anti-Nectin-4).

In a preferred embodiment, the antibody is m290 with a heavy chain as set forth in SEQ ID NO:96 and a light chain as set forth in SEQ ID NO:97.

In certain embodiments, the anti-Nectin-4 antibody comprised in the antibody-payload conjugate or the pharmaceutical composition may be conjugated to any one of the linkers shown in FIGS. 4, 6, 7 or 8 or any one of the linkers disclosed herein. In a preferred embodiment, the anti-Nectin-4 antibody, in particular the anti-Nectin-4 antibody m290, may be conjugated to the linker shown in FIG. 8.

A Nectin-4-positive cancer, as used herein, may be, without limitation Nectin-4-positive pancreatic cancer, lung cancer, bladder cancer or breast cancer. The skilled person is able do determine whether a cancer is a Nectin-4-positve cancer. For example, tumor cells may be isolated in a biopsy and the presence of Nectin-4 may be determined with any method known in the art.

The anti-Nectin-4 antibody-payload conjugate and/or the pharmaceutical composition comprising an anti-Nectin-4 antibody-payload conjugate according to the invention may be administered alone in patients who have previously received a PD-1 or PD-L1 inhibitor in combination with a platinum-based chemotherapeutic agent before or after surgery.

Further, the anti-Nectin-4 antibody-payload conjugate and/or the pharmaceutical composition comprising an anti-Nectin-4 antibody-payload conjugate may be used in conjunction with other therapies that are suitable for the treatment of Nectin-4-positive cancers.

Thus, in a particular embodiment, the invention relates to the antibody-payload conjugate or the pharmaceutical composition for use according to the invention, wherein the antibody-payload conjugate or the pharmaceutical composition is administered in combination with a platinum-based chemotherapeutic agent and/or Pembrolizumab.

It is to be understood that the antibody-payload conjugate or the pharmaceutical composition does not necessarily have to be administered at the same time as the additional therapeutic agent, such as the cisplatin-based chemotherapeutic agent and/or Pembrolizumab. Instead the antibody-payload conjugate or the pharmaceutical composition may be administered with a different schedule and, consequently, on different days as other therapeutic agents that are used for the treatment of the same disease.

In certain embodiments, the antibody-payload conjugate and/or the pharmaceutical composition according to the invention may be used in the treatment of NaPi2b-positive cancers.

That is, in a particular embodiment, the invention relates to the antibody-payload conjugate or the pharmaceutical composition for use according to the invention, wherein the antibody-payload conjugate comprises Upifitamab, and wherein the neoplastic disease is an anti-NaPi2b positive cancer.

For this, it is preferred that the antibody-payload conjugate comprises an anti-NaPi2b antibody as disclosed herein, preferably wherein the anti-NaPi2b antibody is internalized into a target cell upon binding to NaPi2b. In a preferred embodiment, the antibody is Upifitamab with a heavy chain as set forth in SEQ ID NO:98 and a light chain as set forth in SEQ ID NO:99.

As outlined above, in a preferred embodiment, the invention relates to the antibody-payload conjugate according to the invention, wherein the antibody is an antibody, preferably, an IgG antibody comprising at Kabat position 234 an A and/or at Kabat position 235 an A.

Accordingly, in certain embodiments, the antibody is Upifitamab comprising at Kabat position 234 an A and at Kabat position 235 an A with a heavy chain as set forth in SEQ ID NO:100.

In certain embodiments, the antibody is Upifitamab comprising at Kabat position 234 an A and at Kabat position 235 an A with a heavy chain as set forth in SEQ ID NO:100 and a light chain as set forth in SEQ ID NO:101.

In certain embodiments, the anti-NaPi2b antibody comprised in the antibody-payload conjugate or the pharmaceutical composition may be conjugated to any one of the linkers shown in FIGS. 4, 6 or 7 or any one of the linkers disclosed herein. In a preferred embodiment, the anti-NaPi2b antibody, in particular the anti-NaPi2b antibody Upifitamab, may be conjugated to any one of the linkers shown in FIG. 4.

A NaPi2b-positive cancer, as used herein, may be, without limitation NaPi2b-positive lung or ovarian cancer. The skilled person is able do determine whether a cancer is a NaPi2b-positve cancer. For example, tumor cells may be isolated in a biopsy and the presence of NaPi2b may be determined with any method known in the art.

In a particular embodiment, the invention relates to a use of the antibody-payload conjugate according to the invention, or the pharmaceutical composition according to the invention for the manufacture of a medicament for the treatment of a patient suffering from, being at risk of developing, and/or being diagnosed for a neoplastic disease.

As regards the preferred embodiments of the use of the antibody-payload conjugate according to the invention, or the pharmaceutical composition according to the invention for the manufacture of a medicament for the treatment of a patient suffering from, being at risk of developing, and/or being diagnosed for a neoplastic disease, the same applies, mutatis mutandis, as has been set forth above in the context of the composition or the pharmaceutical composition for uses as defined above.

The antibody-payload conjugate or the pharmaceutical composition according to the invention may be administered by any suitable means, including parenteral, intrapulmonary, and intranasal, and, if desired for local treatment, intralesional, intrauterine or intravesical administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

The antibody-payload conjugate or the pharmaceutical composition according to the invention may be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody-payload conjugate or the pharmaceutical composition according to the invention need not be, but is optionally formulated with one or more agents currently used to prevent or treat the disorder in question. The effective amount of such other agents depends on the amount of antibody-payload conjugate present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of the antibody-payload conjugate or the pharmaceutical composition according to the invention (when used alone or in combination with one or more other additional therapeutic agents) will depend on the type of disease to be treated, the type of antibody-payload conjugate, the severity and course of the disease, whether the antibody-linker conjugate is administered for preventive or therapeutic purposes, previous therapy, the patient's clinical history and response to the antibody-linker conjugate, and the discretion of the attending physician. The antibody-payload conjugate or the pharmaceutical composition according to the invention is suitably administered to the patient at one time or over a series of treatments.

The invention also relates to methods of treating a patient suffering from, being at risk of developing, and/or being diagnosed for a neoplastic disease, wherein the ADC or the pharmaceutical composition of the present invention as defined herein is administered to a subject, preferably, in a therapeutically effective amount as defined herein.

As regards the preferred embodiments of the methods for treatment the same applies, mutatis mutandis, as has been set forth above in the context of the composition or the pharmaceutical composition for uses as defined above.

Another aspect of the present invention relates to the linker as such, in particular the peptide linkers disclosed herein.

That is, in a particular embodiment, the invention relates to a linker comprising a first and second payload, wherein the first payload is a topoisomerase I inhibitor which is cell-permeable, preferably a camptothecin cytotoxic molecule which is cell-permeable; and
wherein the second payload a topoisomerase I inhibitor which is not cell-permeable,
preferably a camptothecin cytotoxic molecule which is not cell-permeable,
preferably wherein the linker is for conjugation to an antibody

In another embodiment, the invention relates to a linker according to the invention, wherein the camptothecin is an exatecan or a deruxtecan.

In another embodiment, the invention relates to a linker according to the invention, wherein the second payload has been modified to reduce its cell permeability.

In another embodiment, the invention relates to a linker according to the invention, wherein the second payload has a glycine residue linked to said topoisomerase I inhibitor of the second payload.

In another embodiment, the invention relates to a linker according to the invention, wherein the linker is a peptide linker.

In another embodiment, the invention relates to a linker according to the invention, wherein the first payload and/or second payload are linked to the N- or C-terminus of the peptide linker or to a side-chain of an amino acid residue comprised in the peptide linker; preferably wherein the first payload is linked to the N-terminus of the peptide linker and wherein the second payload is linked to the C-terminus of the peptide linker, or vice versa.

In another embodiment, the invention relates to a linker according to the invention, wherein the linker comprises a primary amine for conjugation to an antibody, preferably wherein the primary amine is comprised in a lysine residue, a lysine mimetic or a lysine derivative; or wherein the primary amine is comprised in an amino acid residue having the structure NH₂—(CH₂)₁₋₁₀—COOH; preferably wherein the amino acid residues are comprised in a peptide linker.

In another embodiment, the invention relates to a linker according to the invention, wherein the linker further comprises at least one positively charged amino acid residue, preferably wherein the at least one positively charged amino acid residue is selected from arginine and/or histidine.

In another embodiment, the invention relates to a linker according to the invention, wherein the linker consists of or comprises the following structure:

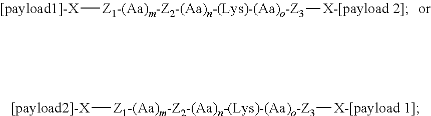

wherein
[payload 1] is said first payload;
[payload 2] is said second payload;
(Aa) is any amino acid residue;
m, n and o may be integers ranging from 0 to 10, preferably 0 to 6, more preferably 0 to 4;
(Lys) is a lysine residue, a lysine mimetic or a lysine derivative;
Z₁₋₃ is either absent or a spacer comprising an alkyl or a heteroalkyl group, preferably wherein the spacer comprises (C_{H}2)₂; and
X is either absent or a self-immolative group, preferably PABC.

In another embodiment, the invention relates to a linker according to the invention, wherein (Aa)ₘ+(Aa)ₙ+(Aa)ₒ is >0, preferably wherein (Aa)ₙ+(Aa)ₒ is >0.

In another embodiment, the invention relates to a linker according to the invention, wherein Z₂ is a dicarboxylic acid linking the N-terminal end of (Aa)ₘ to the N-terminal end of (Aa)ₙ or (Lys); and wherein one payload is directly or indirectly linked to the C-terminal end of (Aa)ₘ and the other payload is directly or indirectly linked to the C-terminal end of (Lys) or (Aa)ₒ, or vice versa.

In another embodiment, the invention relates to a linker according to the invention, wherein the linker consists of or comprises the following structure:

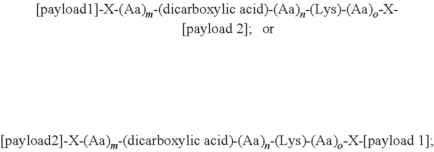

wherein
[payload 1] is said first payload;
[payload 2] is said second payload;
(Aa) is any amino acid residue;
m is an integer ranging from 1 to 10, preferably 1 to 6, more preferably 1 to 4;
n and o may be integers ranging from 0 to 10, preferably 0 to 6, more preferably 0 to 4, wherein n+o is >0;
(Lys) is a lysine residue, a lysine mimetic or a lysine derivative;
(dicarboxylic acid) is dicarboxylic acid linking the N-terminal end of (Aa)ₘ to the N-terminal end of (Aa)ₙ or (Lys); and
X is either absent or a self-immolative group, preferably PABC.

In another embodiment, the invention relates to a linker according to the invention, wherein (Aa)ₙ-(Lys)-(Aa)ₒ comprises the sequence motif Arg-Lys (RK) or His-Lys (HK) (in N->C direction).

In another embodiment, the invention relates to a linker according to the invention, wherein (Aa)ₙ-(Lys)-(Aa)ₒ is or comprises RK or RKAA (in N->C direction).

In another embodiment, the invention relates to a linker according to the invention, wherein the linker comprises or consists of the following structure:

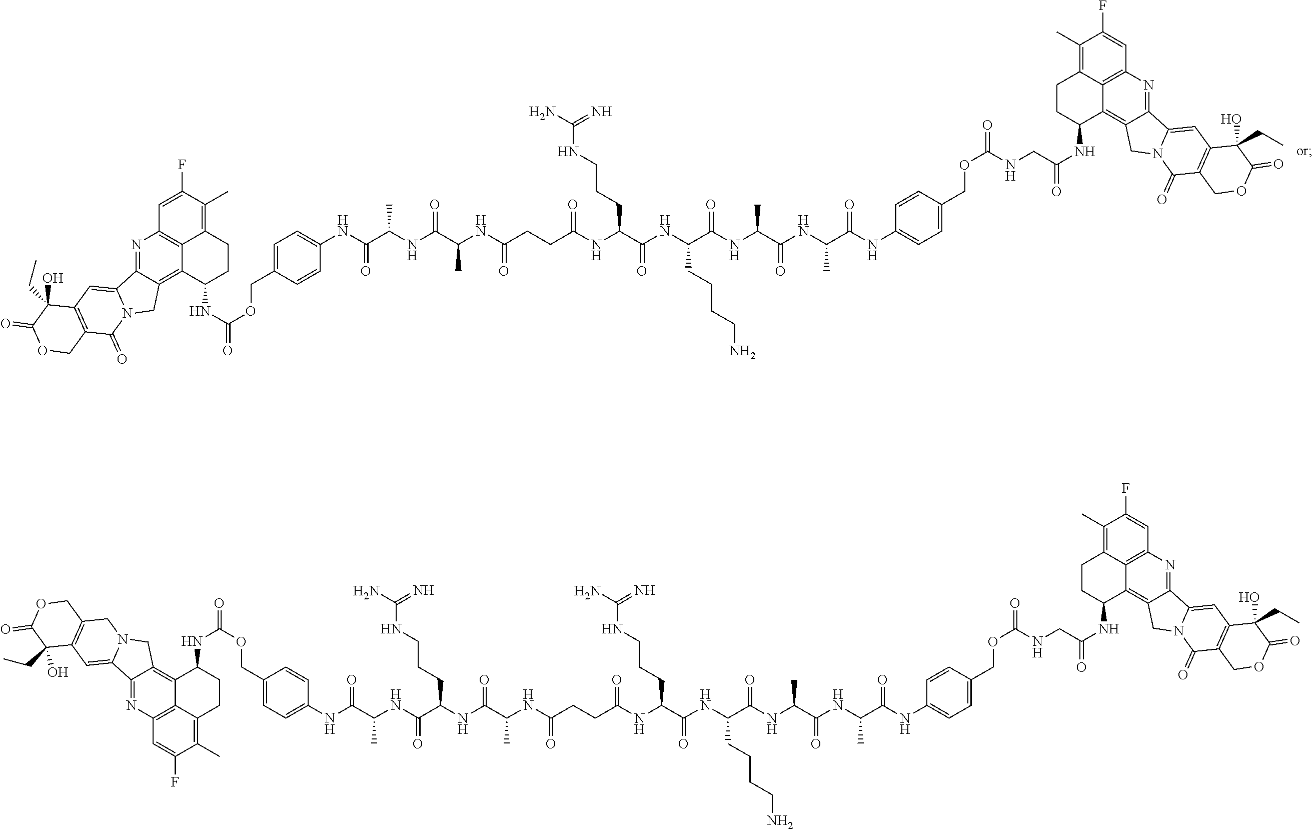

In another embodiment, the invention relates to a linker according to the invention, wherein said linker further comprises a third payload, preferably a toxin or a cytotoxin, more preferably, an auristatin, and even more preferably, an MMAE (Monomethyl auristatin E).

In another embodiment, the invention relates to a linker according to the invention, wherein the linker is a peptide linker and wherein the third payload is linked to the N- or C-terminus of the peptide linker or wherein the third payload is linked to a side chain of an amino acid residue comprised in the peptide linker.

In another embodiment, the invention relates to a linker according to the invention, wherein two payloads are linked to the same functional group of the peptide linker, preferably via a chemical linker comprising a disubstituted amine.

In another embodiment, the invention relates to a linker according to the invention, wherein the linker consists of or comprises the structure:

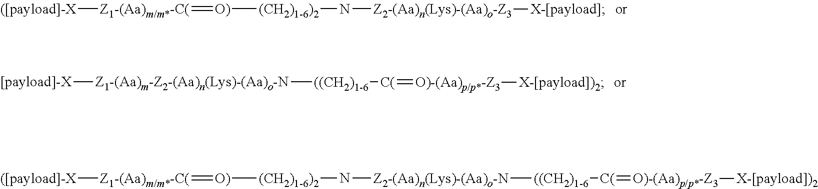

wherein
[payload] is each independently a payload selected from the first, second and third payload, wherein the linker comprises all three payloads;
(Aa) is any amino acid residue;
m, m*, n, o, p and p* may be integers ranging from 0 to 10, preferably 0 to 6, more preferably 0 to 4;
(Lys) is a lysine residue, a lysine mimetic or a lysine derivative;
Z₁₋₃ is either absent or a spacer comprising an alkyl or a heteroalkyl group, preferably wherein the spacer comprises (CH₂)₂, even more preferably wherein Z₂ is a dicarboxylic acid linker; and
X is either absent or a self-immolative group, preferably PABC.

In another embodiment, the invention relates to a linker according to the invention, wherein (Aa)ₙ+(Aa)ₒ is >0.

In another embodiment, the invention relates to a linker according to the invention, wherein the linker consists of or comprises the structure:

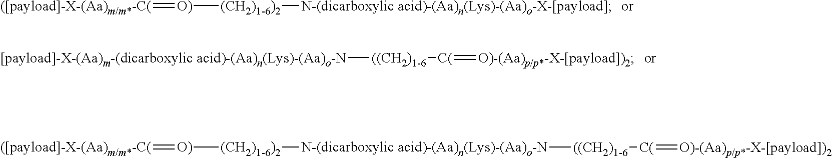

wherein
[payload] is each independently a payload selected from the first, second and third payload, wherein the linker comprises all three payloads;
(Aa) is any amino acid residue;
m, m*, p and p* are integers ranging from 1 to 10, preferably 1 to 6, more preferably 1 to 4;
n and o may be integers ranging from 0 to 10, preferably 0 to 6, more preferably 0 to 4, wherein n+o is >0;
(Lys) is a lysine residue, a lysine mimetic or a lysine derivative;
(dicarboxylic acid) is dicarboxylic acid linking the N-terminal end of (Aa)ₘ or the disubstituted amine (N) to the N-terminal end of (Aa)ₙ or (Lys); and
X is either absent or a self-immolative group, preferably PABC.

In another embodiment, the invention relates to a linker according to the invention, wherein (Aa)ₙ-(Lys)-(Aa)ₒ comprises the sequence motif Arg-Lys (RK) or His-Lys (HK) (in N->C direction).

In another embodiment, the invention relates to a linker according to the invention, wherein (Aa)ₙ-(Lys)-(Aa)ₒ is or comprises RK or RKAA (in N->C direction).

In another embodiment, the invention relates to a linker according to the invention, wherein the linker comprises or consists of the following structure:

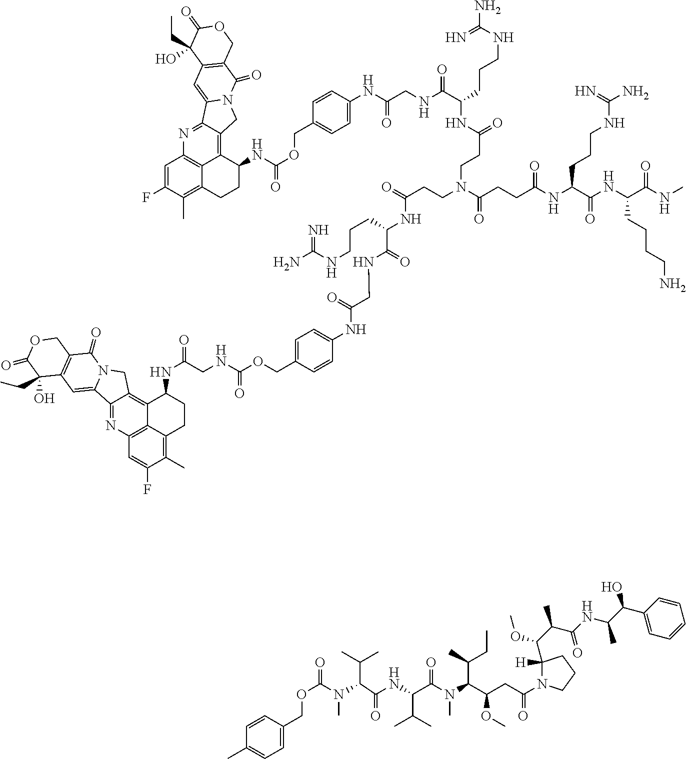

As regards the exact composition of the linker as well as preferred embodiments thereof, same applies, mutatis mutandis, as has been set forth above in the context of the ADC of the present invention.

Another aspect of the present invention relates to a method for the preparation of the ADC according to the invention. ADCs may the prepared using the methods disclosed in WO 2023/161291, which is fully incorporated herein by reference.

In a particular embodiment, the invention relates to a method for the preparation of an antibody-drug conjugate comprising a step of conjugating a peptide linker according to the invention to an antibody.

That is, any of the peptide linkers comprising at least a first and second topoisomerase I inhibitor as disclosed herein may be conjugated to an antibody. In particular, any of the amine-comprising peptide linkers disclosed herein may be conjugated to a glutamine residue of an antibody via a transglutaminase. As disclosed elsewhere herein, the glutamine residue to which the peptide linker is conjugated may be an endogenous glutamine residue (e.g., Q295 of an IgG antibody) or may be a glutamine residue that has been introduced into the antibody by molecular engineering.

In a particular embodiment, the invention relates to a method for the conjugation of a peptide linker according to the invention using a transglutaminase (TG), the method comprising a) mixing the antibody, the peptide linker and a transglutaminase (TG) within a fluid, thereby conjugating the linker-payload to the antibody in one step under the catalyzing effect of the TG, and b) extracting the conjugate obtained in step a) from the fluid.

Accordingly, the present invention further encompasses methods for conjugating peptide linkers according to the invention to an antibody by means of a transglutaminase in a one-step reaction. For that, an antibody may be mixed with the peptide linker according to the invention and a transglutaminase within a fluid. A “fluid”, within the meaning of the present invention is a liquid. Preferably, the liquid is an aqueous solution, even more preferably a buffered aqueous solution.

The peptide linker according to the invention may be mixed with the antibody and the transglutaminase by mixing a solution comprising said peptide linker with a solution comprising an antibody and a solution comprising the transglutaminase. Alternatively, solutions individually comprising the peptide linker, the antibody and the transglutaminase may be added to an aqueous solution. In particular, each component may be added to the aqueous solution at a defined concentration. The peptide linker according to the invention is conjugated to the antibody under the catalyzing effect of the transglutaminase. That is, the individual components may be mixed under conditions that are suitable for an efficient conjugation of the peptide linker to the antibody. Such conditions are defined elsewhere herein.

In a second method step, the obtained antibody-payload conjugates have to be removed from the liquid. The skilled person is aware of methods to isolate antibody-payload conjugates from an aqueous solution. Further, the skilled person is aware of methods to separate antibody-payload conjugates from unconjugated antibodies or peptide linkers or from incompletely conjugated antibodies. For example, antibody payload conjugates according to the invention may be isolated from the mixture by HPLC.

It is to be understood that “extracting the conjugate from the fluid” is synonymous with “isolating the conjugate from the mixture”. That is, a conjugate may also be extracted by removing the transglutaminase and unconjugated antibody and peptide linker from the fluid.

The peptide linker that is used in the method according to the invention may be any one of the peptide linkers disclosed herein, in particular any peptide linker falling within the definition provided herein above or any peptide linker shown in the experimental examples.

Thus, in a particular embodiment, the invention relates to the method according to the invention, wherein the peptide linker is the peptide linker of the invention.

Further, the antibody may be an antibody as defined in more detail elsewhere herein, i.e., for the antibody-payload conjugate according to the invention.

In particular, the peptide linker may comprise an amino acid sequence as set forth in SEQ ID NOs:1-29 or 82-93. Furthermore, the linker may be any one of the linkers shown in FIGS. 4, 6, 7 or 8.

That is, in a particular embodiment, the invention relates to the method according to the invention, wherein the peptide linker is conjugated to a glutamine residue comprised in the antibody via a primary amine comprised in an amino acid residue of the peptide linker.

In a particular embodiment, the invention relates to the method according to the invention, wherein the antibody is an antibody fragment, as defined elsewhere herein.

In a particular embodiment, the invention relates to the method according to the invention, wherein the antibody is an IgA, IgD, IgE, IgG or IgM antibody.

In a particular embodiment, the invention relates to the method according to the invention, wherein the peptide linker is conjugated to a glutamine residue comprised in an Fc domain of the antibody.

In a particular embodiment, the invention relates to the method according to the invention, wherein the glutamine residue to which the peptide linker is conjugated is glutamine residue Q295 (EU numbering) of the C_{H}2 domain of an IgG antibody.

In a particular embodiment, the invention relates to the method according to the invention, wherein the glutamine residue to which the peptide linker is conjugated has been introduced into the heavy or light chain of the antibody by molecular engineering.

In a particular embodiment, the invention relates to the method according to the invention, wherein the glutamine residue that has been introduced into the heavy or light chain of the antibody by molecular engineering is N297Q (EU numbering) of the C_{H}2 domain of an aglycosylated IgG antibody.

In a particular embodiment, the invention relates to the method according to the invention, wherein the glutamine residue that has been introduced into the heavy or light chain of the antibody by molecular engineering is comprised in a peptide that has been (a) integrated into the heavy or light chain of the antibody or (b) fused to the N- or C-terminal end of the heavy or light chain of the antibody.

In a particular embodiment, the invention relates to the method according to the invention, wherein the peptide comprising the Gln residue has been fused to the C-terminal end of the heavy chain of the antibody.

In a particular embodiment, the invention relates to the method according to the invention, wherein the antibody is a glycosylated IgG antibody.

In a particular embodiment, the invention relates to the method according to the invention, wherein the IgG antibody is glycosylated at residue N297 (EU numbering) of the C_{H}2 domain.

In a particular embodiment, the invention relates to the method according to the invention, wherein the antibody is selected from the group consisting of: m290, Trastuzumab, Brentuximab, Gemtuzumab, Inotuzumab, Avelumab, Cetuximab, Rituximab, Daratumumab, Pertuzumab, Vedolizumab, Ocrelizumab, Tocilizumab, Ustekinumab, Golimumab, Obinutuzumab, Sacituzumab, Belantamab, Polatuzumab, Enfortumab, Endrecolomab, Gemtuzumab,Loncastuximab, Mecbotamab, Adecatumumab, D93, Gatipotuzumab, Labetuzumab, Tusamitamab, Upifitamab, Lifastuzumab, Mirvetuximab, Sofituzumab, Anetumab, Tisotumab, Cofituzumab, Praluzatamab, Ladriatuzumab, Belantamab, Patritumab, Cetuximab, Nimotuzumab, Matuzumab, Portuzumab, Citatuzumab, Tucotuzumab and Endrecolomab; and/or
wherein the antibody specifically binds to an antigen selected from the group consisting of: CD30, Her2/neuCD33, CD22, PD-L1, EGFR, CD20, CD38, HER2, Integrin α4β7, CD20, IL-6-R, IL-12, IL-23, TNFα, CD20, Trop-2, BCMA, CD79b, Nectin-4, EpCAM, CD33, CD19, AXL, dn-collagen, TA-MUC1, carcinoembryonic cell adhesion molecule 5, CEACAM5, NaPi2b, FRα, MUC16, mesothelin, TF, CD166, LIV-1, ERBB3, EGFR, and TACSTD1, preferably, CD30, Her2/neu, CD22, CD79b, Nectin-4, Trop-2 and BCMA, more preferably, CD79b, Her2/neu, and Nectin-4.

In a particular embodiment, the invention relates to the method according to the invention, wherein the peptide linker is conjugated to a γ-carboxamide group of a Gln residue comprised in the antibody.

In a particular embodiment, the invention relates to the method according to the invention, wherein the peptide linker is suitable for conjugation to a glycosylated antibody with a conjugation efficiency of at least 20%, 30%, 40%, 50%, 60%, 70%, 75%, 80%, 85%, 90% or 95%.

That is, in certain embodiments, the peptide linker according to the invention may be conjugated to a glycosylated antibody with an efficiency of at least 20%, 30%, 40%, 50%, 60%, 70%, 75%, 80%, 85%, 90% or 95%. In a preferred embodiment, the peptide linker according to the invention may be conjugated to a glycosylated antibody with an efficiency of at least 70%. In another preferred embodiment, the peptide linker according to the invention may be conjugated to a glycosylated antibody with an efficiency of at least 75%. In another preferred embodiment, the peptide linker according to the invention may be conjugated to a glycosylated antibody with an efficiency of at least 80%. In another preferred embodiment, the peptide linker according to the invention may be conjugated to a glycosylated antibody with an efficiency of at least 85%. In another preferred embodiment, the peptide linker according to the invention may be conjugated to a glycosylated antibody with an efficiency of at least 90%. In another preferred embodiment, the peptide linker according to the invention may be conjugated to a glycosylated antibody with an efficiency of at least 95%. Preferably, the glycosylated antibody is a glycosylated IgG antibody, more preferably an IgG antibody that is glycosylated at residue N297 (EU numbering).

The skilled person is aware of methods to determine the conjugation efficiency of an antibody with a specific peptide linker. For example, the conjugation efficiency may be determined as described herein. That is, an antibody, in particular an IgG1 antibody, may be incubated at a concentration of 1-5 mg/mL with 5-20 eq molar equivalents of a linker and 3-6 U of a microbial transglutaminase per mg of antibody in a suitable buffer for 20-48 hours at 37° C. or as described in Example 1. After the incubation period, the conjugation efficiency may be determined by LC-MS analysis under reducing conditions. The microbial transglutaminase may be an MTG from Streptomyces mobaraensis that is, for example, available from Zedira (Germany). A suitable buffer may be a Tris, MOPS, HEPES, PBS or BisTris buffer. However, it is to be understood that the choice of the buffer system may vary and depend to a large extent on the chemical properties of the linker. However, the skilled person is capable of identifying the optimal buffer conditions based on the disclosure of the present invention. Alternatively, the conjugation efficiency may be determined as described in Spycher et al. (Dual, Site-Specific Modification of Antibodies by Using Solid-Phase Immobilized Microbial Transglutaminase, ChemBioChem 2019 18(19):1923-1927) and analyzed as in Benjamin et al. (Thiolation of Q295: Site-Specific Conjugation of Hydrophobic Payloads without the Need for Genetic Engineering, Mol. Pharmaceutics 2019, 16: 2795-2807).

In certain embodiments, antibodies may be conjugated by incubating 5 mg/ml of native, glycosylated monoclonal antibody for 24 hours at 37° C. in 50 mM Tris pH 7.6 with a microbial transglutaminase (MTG, Zedira) at a concentration of 5-10 U/mg antibody and 5 molar equivalents of the indicated linker-payload in a rotating thermomixer. However, it is to be understood that the conditions, in particular the buffer conditions and the peptide linker concentration may be adjusted depending on the properties of the payload(s). However, the skilled person is able to identify the optimal reaction conditions based on the teaching provided herein.

In a particular embodiment, the invention relates to the method according to the invention, wherein the transglutaminase is a microbial transglutaminase (MTG).

The transglutaminase for use in the method of the present invention may be any transglutaminase that is suitable for conjugating the peptide linker of the invention to an antibody. The transglutaminase may be of any origin, e.g., the transglutaminase may be of bacterial, archaeal or eukaryotic origin.

In certain embodiments, the transglutaminase may be a mammalian transglutaminase, including human transglutaminases. In certain embodiments, the transglutaminase may be a microbial transglutaminase, including bacterial and fungal transglutaminases.

In a particular embodiment, the invention relates to the method according to the invention, wherein the microbial transglutaminase is derived from a Streptomyces species, in particular Streptomyces mobaraensis.

That is, the microbial transglutaminase used in the method of the invention may be derived from a Streptomyces species, in particular from Streptomyces mobaraensis, preferentially with a sequence identity of 80% to the native enzyme. Accordingly, the MTG may be a native enzyme or may be an engineered variant of a native enzyme.

One such microbial transglutaminase is commercially available from Zedira (Germany). It is recombinantly produced in E. coli. Streptomyces mobaraensis transglutaminase has an amino acid sequence as disclosed in SEQ ID NO:78. S. mobaraensis MTG variants with other amino acid sequences have been reported and are also encompassed by this invention (SEQ ID NO:79 and 80).

One such microbial transglutaminase could also be the MTG-TX variant from S. mobaraensis described in Jin et al. 2016, Journal of Molecular Catalysis B: Enzymatic, which exhibits high-salt-resistance and a broad range of pH and temperature stability.

In another embodiment, a microbial transglutaminase from Streptomyces ladakanum (formerly known as Streptoverticillium ladakanum) may be used. Streptomyces ladakanum transglutaminase (U.S. Pat. No. 6,660,510 B2) has an amino acid sequence as disclosed in SEQ ID NO:81.

Both of the above transglutaminases may be sequence modified. In several embodiments, transglutaminases may be used which have 80%, 85%, 90% or 95% or more sequence identity with any one of SEQ ID NO:78-81.

Another suitable microbial transglutaminase is commercially from Ajinomoto, called ACTIVA TG. In comparison to the transglutaminase from Zedira, ACTIVA TG lacks 4 N-terminal amino acids, but has similar activity.

Further microbial transglutaminases which may be used in the context of the present invention are disclosed in Kieliszek and Misiewicz (Folia Microbiol (Praha). 2014; 59(3): 241-250), WO 2015/191883 A1, WO 2008/102007 A1 and US 2010/0143970, the contents of which is fully incorporated herein by reference.

In certain embodiments, a mutant variant of a microbial transglutaminase may be used for the conjugation of a linker to an antibody. That is, the microbial transglutaminase that is used in the method of the present invention may be a variant of S. mobaraensis transgluatminase as set forth in SEQ ID NOs: 78 or 79. In certain embodiments, the recombinant S. morabaensis transglutaminase as set forth in SEQ ID NO:78 may comprise the mutation G254D. In certain embodiments, the recombinant S. morabaensis transglutaminase as set forth in SEQ ID NO:78 may comprise the mutations G254D and E304D. In certain embodiments, the recombinant S. morabaensis transglutaminase as set forth in SEQ ID NO:78 may comprise the mutations D8E and G254D. In certain embodiments, the recombinant S. morabaensis transglutaminase as set forth in SEQ ID NO:78 may comprise the mutations E124A and G254D. In certain embodiments, the recombinant S. morabaensis transglutaminase as set forth in SEQ ID NO:78 may comprise the mutations A216D and G254D. In certain embodiments, the recombinant S. morabaensis transglutaminase as set forth in SEQ ID NO:78 may comprise the mutations G254D and K331T.

In a particular embodiment, the invention relates to the method according to the invention, wherein the transglutaminase is added to the conjugation reaction at a concentration of less than 200 U/mg antibody.

Microbial transglutaminase may be added to the conjugation reaction at any concentration that allows efficient conjugation of an antibody with a linker. In certain embodiments, the concentration of microbial transglutaminase in a conjugation reaction may depend on the amount of antibody used in the same reaction. For example, a microbial transglutaminase may be added to the conjugation reaction at a concentration of less than 200 U/mg antibody, 150 U/mg antibody 100 U/mg antibody, 90 U/mg antibody, 80 U/mg antibody, 70 U/mg antibody, 60 U/mg antibody, 50 U/mg antibody, 40 U/mg antibody, 30 U/mg antibody, 20 U/mg antibody 10 U/mg antibody or 6 U/mg antibody.

In certain embodiments a microbial transglutaminase may be added to the conjugation reaction at a concentration of 1 U/mg antibody. In certain embodiments a microbial transglutaminase may be added to the conjugation reaction at a concentration of 3 U/mg antibody. In certain embodiments a microbial transglutaminase may be added to the conjugation reaction at a concentration of 5 U/mg antibody. In certain embodiments a microbial transglutaminase may be added to the conjugation reaction at a concentration of 6 U/mg antibody. In certain embodiments a microbial transglutaminase may be added to the conjugation reaction at a concentration of 7.5 U/mg antibody. In certain embodiments a microbial transglutaminase may be added to the conjugation reaction at a concentration of 10 U/mg antibody.

In certain embodiments a microbial transglutaminase may be added to the conjugation reaction at a concentration of 1-100 U/mg antibody. In certain embodiments a microbial transglutaminase may be added to the conjugation reaction at a concentration of 3-50 U/mg antibody. In certain embodiments a microbial transglutaminase may be added to the conjugation reaction at a concentration of 5-25 U/mg antibody.

In certain embodiments a microbial transglutaminase may be added to the conjugation reaction at a concentration of 1-20 U/mg antibody, preferably at a concentration of 3-15 U/mg antibody, more preferably at a concentration of 5-10 U/mg antibody.

Preferably, the transglutaminase for use in the method of the invention is a microbial transglutaminase. However, it is to be noted that an equivalent reaction may be carried out by an enzyme comprising transglutaminase activity that is of a non-microbial origin. Accordingly, also the antibody-payload conjugates according to the invention may be generated with an enzyme comprising transglutaminase activity that is of a non-microbial origin.

In a particular embodiment, the invention relates to the method according to the invention, wherein the antibody is added to the conjugation reaction at a concentration of 0.1-50 mg/mL.

The antibody may be added to the conjugation reaction at any concentration that is suitable for obtaining efficient conjugation of the antibody. However, it is preferred that the antibody is added to the conjugation reaction at a concertation ranging from 0.1-50 mg/ml. That is, in a particular embodiment, the invention relates to the method according to the invention, wherein the antibody is added to the conjugation reaction at a concentration of 0.1-50 mg/mL, preferably 0.25-25 mg/mL, more preferably 0.5-12.5 mg/mL, even more preferably 1-10 mg/mL, even more preferably 2-7.5 mg/mL, most preferably about 5 mg/mL.

Alternatively, the antibody may be added to the conjugation reaction at a concertation ranging from 1-20 mg/ml, preferably from 2.5-20 mg/mL, more preferably from 5-20 mg/mL, most preferably from 5-17 mg/mL.

In a particular embodiment, the invention relates to the method according to the invention, wherein the antibody is contacted with 2-100 molar equivalents of peptide linker.

To obtain efficient conjugation, it is preferred that the linker is added to the antibody in molar excess. That is, in certain embodiments, the antibody is mixed with at least 2, 5, 10, 20, 30, 40, 50, 60, 70, 80, 90 or 100 molar equivalents of a linker.

That is, in a particular embodiment, the invention relates to the method according to the invention, wherein the antibody is contacted with 2-100 molar equivalents of linker, preferably 2-80 molar equivalents of linker, more preferably 2-70 molar equivalents of linker, even more preferably 2-60 molar equivalents of linker, even more preferably 2-50 molar equivalents of linker, even more preferably 2-40 molar equivalents of linker, even more preferably 2-30 molar equivalents of linker, even more preferably 2 to 25 molar equivalents of linker, even more preferably 2-20 molar equivalents of linker, even more preferably 2-15 molar equivalents of linker, most preferably 2-10 molar equivalents of linker.

Alternatively, the antibody may be contacted with 2.5-100 molar equivalents of linker, preferably 2.5-80 molar equivalents of linker, more preferably 2.5-70 molar equivalents of linker, even more preferably 2.5-60 molar equivalents of linker, even more preferably 2.5-50 molar equivalents of linker, even more preferably 2.5-40 molar equivalents of linker, even more preferably 2.5-30 molar equivalents of linker, even more preferably 2.5-20 molar equivalents of linker, even more preferably 2.5-15 molar equivalents of linker, even more preferably 2.5-10 molar equivalents of linker, most preferably 2.5-8 molar equivalents of linker.

Alternatively, the antibody may be contacted with 5-100 molar equivalents of linker, preferably 5-80 molar equivalents of linker, more preferably 5-70 molar equivalents of linker, even more preferably 5-60 molar equivalents of linker, even more preferably 5-50 molar equivalents of linker, even more preferably 5-40 molar equivalents of linker, even more preferably 5-30 molar equivalents of linker, even more preferably 5-20 molar equivalents of linker, even more preferably 5-15 molar equivalents of linker, most preferably 5-10 molar equivalents of linker.

In a particular embodiment, the invention relates to the method according to the invention, wherein the conjugation reaction is carried out in a buffered solution.

The method according to the invention is preferably carried out at a pH ranging from 5 to 10. Thus, in a preferred embodiment, the invention relates to a method according to the invention, wherein the conjugation of the linker to the antibody is achieved at a pH ranging from 5 to 10, preferably at a pH ranging from 6 to 9, more preferably at a pH ranging from 6 to 8.5, even more preferably at a pH ranging from 6.5 to 8, most preferably at a pH ranging from 6.6 to 7.6.

In certain embodiments, the invention relates to a method according to the invention, wherein the conjugation of the linker to the antibody is achieved at pH 6.6.

In certain embodiments, the invention relates to a method according to the invention, wherein the conjugation of the linker to the antibody is achieved at pH 7.6.

The method of the invention may be carried out in any buffer that is suitable for the conjugation of the payload to the linker. Buffers that are suitable for the method of the invention include, without limitation, Tris, MOPS, HEPES, PBS or BisTris buffer. The concentration of the buffer depends, amongst others, on the concentration of the antibody and/or the linker and may range from 10-1000 mM, 10-500 mM, 10-400 mM, 10 to 250 mM, 10 to 150 mM or 10 to 100 mM. Further, the buffer may comprise any salt concentration that is suitable for carrying out the method of the invention. For example, the buffer used in the method of the invention may have a salt concentration≤250 mM, ≤200 mM, ≤150 mM, ≤140 mM, ≤130 mM, ≤120 mM, ≤110 mM, ≤100 mM, ≤90 mM, ≤80 mM, ≤70 mM, ≤60 mM, ≤50 mM, ≤40 mM, ≤30 mM, ≤20 mM or ≤10 mM or no salts.

That is, in a particular embodiment, the invention relates to the method according to the invention, wherein the buffered solution comprises
a) a pH ranging from 5 to 10; and/or
b) a buffer concentration ranging from 10 to 1000 mM; and/or
c) a salt concentration below 250 mM.

In a preferred embodiment, the invention relates to the method according to the invention, wherein the buffered solution comprises
a) a pH ranging from 6 to 9; and/or
b) a buffer concentration ranging from 10 to 1000 mM; and/or
c) a salt concentration below 250 mM.

In a more preferred embodiment, the invention relates to the method according to the invention, wherein the buffered solution comprises
a) a pH ranging from 6 to 8; and/or
b) a buffer concentration ranging from 10 to 500 mM; and/or
c) a salt concentration below 150 mM.

In an even more preferred embodiment, the invention relates to the method according to the invention, wherein the buffered solution comprises
a) a pH ranging from 6 to 8; and/or
b) a buffer concentration ranging from 10 to 200 mM; and/or
c) a salt concentration below 50 mM.

In a preferred embodiment, the method of the invention is carried out in 50 mM Tris (pH 7.6), preferably without salts.

In another preferred embodiment, the method of the invention is carried out in 50 mM BisTris (pH 6.6), preferably without salts.

In another preferred embodiment, the method of the invention is carried out in 50 mM BisTris (pH 7.5), preferably without salts.

It has to be noted that the optimal reaction conditions (e.g. pH, buffer, salt concentration) may vary between payloads and to some degree depend on the physicochemical properties of the linkers and/or payloads. However, no undue experimentation is required by the skilled person to identify reaction conditions that are suitable for carrying out the method of the invention.

It is to be understood that the application encompasses any combination of the above-disclosed linker, antibody, MTG and/or buffer concentrations.

In certain embodiments, the invention relates to the methods according to the invention, wherein the antibody is contacted with 2-80 molar equivalents of the linker; and/or wherein the microbial transglutaminase is added to the conjugation reaction at a concentration ranging from 1-20 U/mg antibody and, optionally, wherein the antibody is added to the conjugation reaction at a concentration ranging from 0.1-20 mg/mL.

In a preferred embodiment, the invention relates to the methods according to the invention, wherein the antibody is contacted with 2-50 molar equivalents of the linker; and/or wherein the microbial transglutaminase is added to the conjugation reaction at a concentration ranging from 1-15 U/mg antibody and, optionally, wherein the antibody is added to the conjugation reaction at a concentration ranging from 1-20 mg/mL.

In a more preferred embodiment, the invention relates to the methods according to the invention, wherein the antibody is contacted with 2-30 molar equivalents of the linker; and/or wherein the microbial transglutaminase is added to the conjugation reaction at a concentration ranging from 2-15 U/mg antibody and, optionally, wherein the antibody is added to the conjugation reaction at a concentration ranging from 2.5-20 mg/mL.

In an even more preferred embodiment, the invention relates to the methods according to the invention, wherein the antibody is contacted with 2-20 molar equivalents of the linker; and/or wherein the microbial transglutaminase is added to the conjugation reaction at a concentration ranging from 5-15 U/mg antibody and, optionally, wherein the antibody is added to the conjugation reaction at a concentration ranging from 2.5-20 mg/mL.

In an even more preferred embodiment, the invention relates to the methods according to the invention, wherein the antibody is contacted with 2-15 molar equivalents of the linker; and/or wherein the microbial transglutaminase is added to the conjugation reaction at a concentration ranging from 5-15 U/mg antibody and, optionally, wherein the antibody is added to the conjugation reaction at a concentration ranging from 5-20 mg/mL.

In a most preferred embodiment, the invention relates to the methods according to the invention, wherein the antibody is contacted with 2.5-12.5 molar equivalents of the linker; and/or wherein the microbial transglutaminase is added to the conjugation reaction at a concentration ranging from 5-15 U/mg antibody and, optionally, wherein the antibody is added to the conjugation reaction at a concentration ranging from 5-20 mg/mL.

In another preferred embodiment, the invention relates to the methods according to the invention, wherein the antibody is contacted with 2-20 molar equivalents of the linker; and/or wherein the microbial transglutaminase is added to the conjugation reaction at a concentration ranging from 5-15 U/mg antibody and, optionally, wherein the antibody is added to the conjugation reaction at a concentration ranging from 2.5-20 mg/mL.

It is to be noted that the specific reaction mixtures disclosed above may be freely combined with any of the buffer conditions disclosed herein. However, it is preferred that the specific components as defined above are mixed at a pH ranging from 6 to 8.

In a particular embodiment, the invention relates to an antibody-payload conjugate which has been produced with the method according to the invention.

That is, the invention further relates to an antibody-linker conjugate which has been generated with any of the aforementioned method steps.

In the present invention, the subject is, in a preferred embodiment, a mammal such as a dog, cat, pig, cow, sheep, horse, rodent, e.g., rat, mouse and guinea pig, or a primate, e.g., gorilla, chimpanzee and a human. In a most preferable embodiment, the subject is a human.

Other aspects and advantages of the invention will be described in the following examples, which are given for purposes of illustration and not by way of limitation. Each publication, patent, patent application or other document cited in this application is hereby incorporated by reference in its entirety.

List of embodiments:
P1. An antibody-drug conjugate (ADC) having the formula A-L, wherein A is an antibody or an antibody fragment and wherein L is a linker, said linker comprising:
   as a first payload a topoisomerase I inhibitor which is cell-permeable, preferably a camptothecin cytotoxic molecule which is cell-permeable; and
   as a second payload a topoisomerase I inhibitor which is not cell-permeable, preferably a camptothecin cytotoxic molecule which is not cell-permeable.
P2. The ADC according to embodiment 1, wherein the linker is a peptide linker.
P3. The ADC according to embodiment 1 or 2, wherein the first payload and/or second payload are linked to the N- or C-terminus of the peptide linker or to a side-chain of an amino acid residue comprised in the peptide linker; preferably
   wherein the first payload is linked to the N-terminus of the peptide linker and wherein the second payload is linked to the C-terminus of the peptide linker, or vice versa.
P4. The ADC according to any one of embodiments 1 to 3, wherein the linker consists or comprises the following structure (in N->C direction):

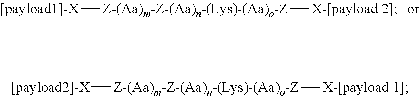

wherein
[payload 1] is said first payload;
[payload 2] is said second payload;
(Aa) is any amino acid residue;
m, n and o may be integers ranging from 0 to 10, preferably 0 to 6, more preferably 0 to 4;
(Lys) is a lysine residue, a lysine mimetic or a lysine derivative;
Z is either absent or a spacer comprising an alkyl or a heteroalkyl group, preferably wherein the spacer is (CH2)₂; and
X is either absent or a self-immolative group, preferably PABC.
P5. The ADC according to any one of embodiments 1 to 4, wherein said ADC comprises more than one first payloads and/or more than one second payloads.
P6. The ADC according to any one of embodiments 1 to 5, wherein the camptothecin is an exatecan or a deruxtecan.
P7. The ADC according to any one of embodiments 1 to 6, wherein the second payload has a glycine residue linked to said topoisomerase I inhibitor of the second payload.
P8. The ADC according to any one of embodiments 1 to 7, wherein the linker comprises or consists of the following structure (in N->C direction):

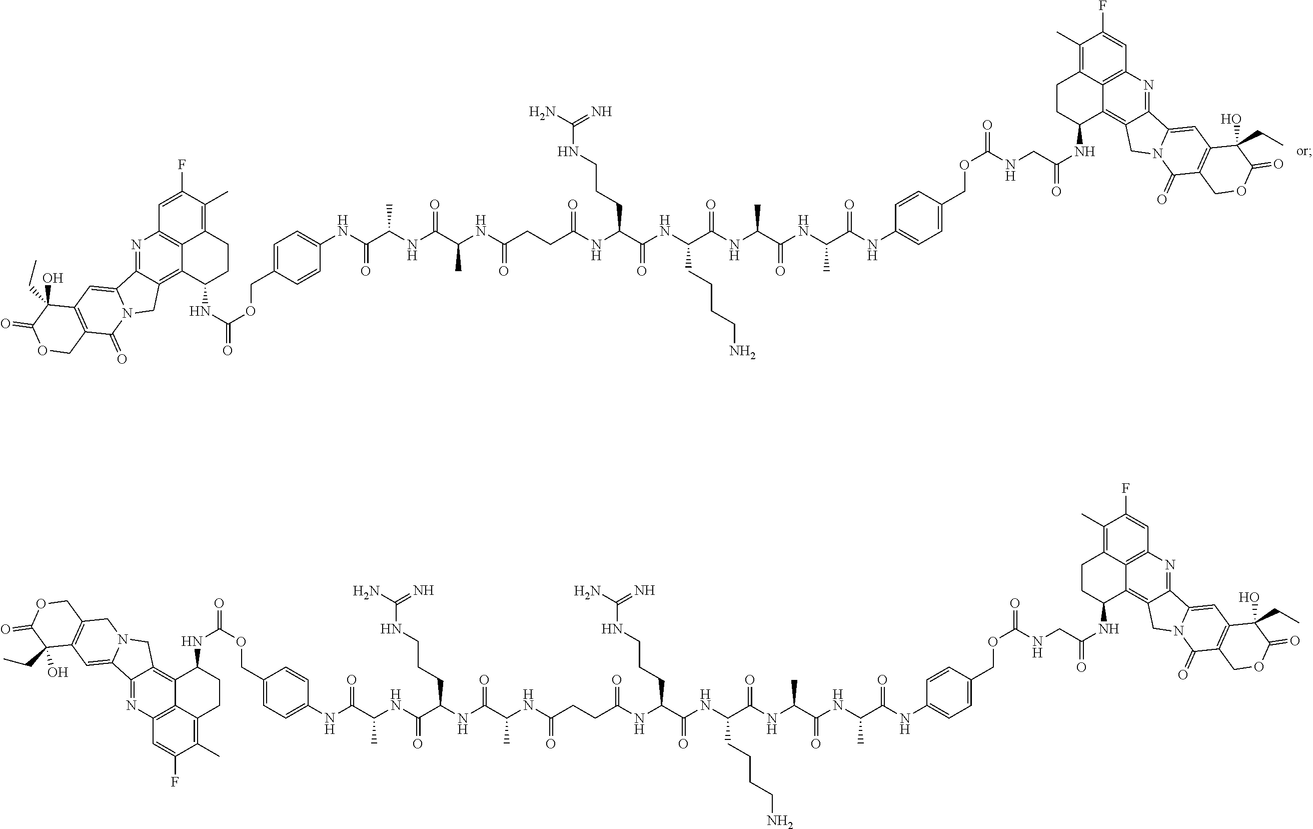

P9. The ADC according to any one of embodiments 1 to 8,
   wherein the antibody is selected from the group consisting of: Trastuzumab, Brentuximab, Gemtuzumab, Inotuzumab, Avelumab, Cetuximab, Rituximab, Daratumumab, Pertuzumab, Vedolizumab, Ocrelizumab, Tocilizumab, Ustekinumab, Golimumab, Obinutuzumab, Sacituzumab, Belantamab, Polatuzumab, Enfortumab, Endrecolomab, Gemtuzumab,Loncastuximab, Mecbotamab, Adecatumumab, D93, Gatipotuzumab, Labetuzumab, Tusamitamab, Upifitamab, Lifastuzumab, Mirvetuximab, Sofituzumab, Anetumab, Tisotumab, Cofituzumab, Praluzatamab, Ladriatuzumab, Belantamab, Patritumab, Cetuximab, Nimotuzumab, Matuzumab, Portuzumab, Citatuzumab, Tucotuzumab and Endrecolomab; and/or
   wherein the antibody specifically binds to an antigen selected from the group consisting of: CD30, Her2/neuCD33, CD22, PD-L1, EGFR, CD20, CD38, HER2, Integrin α4β7, CD20, IL-6-R, IL-12, IL-23, TNFα, CD20, Trop-2, BCMA, CD79b, Nectin-4, EpCAM, CD33, CD19, AXL, dn-collagen, TA-MUC1, carcinoembryonic cell adhesion molecule 5, CEACAM5, NaPi2b, FRα, MUC16, mesothelin, TF, CD166, LIV-1, ERBB3, EGFR, and TACSTD1, preferably, CD30, Her2/neu, CD22, CD79b, Nectin-4, Trop-2 and BCMA, more preferably, CD79b, Her2/neu, and Nectin-4.
P10. The ADC according to any one of embodiments 1 to 9, wherein said ADC consists of two first payloads and two second payloads (drug-to-antibody ratio of 4 “DAR4”).
P11. The ADC according to any one of embodiments 1 to 9, wherein said linker further comprises a third payload, preferably a toxin or a cytotoxin, more preferably, an auristatin, and even more preferably, an MMAE (Monomethyl auristatin E).
P12. A pharmaceutical composition comprising the ADC according to any one of embodiments 1 to 11 and at least one pharmaceutically acceptable ingredient.
P13. The ADC according to any one of embodiments 1 to 11 for use in a method of treating a patient suffering from, being at risk of developing, and/or being diagnosed for a neoplastic disease.
P14. The ADC according to any one of embodiments 1 to 11 and 13 or the pharmaceutical composition of embodiment 12,
   wherein the antibody-payload conjugate comprises Trastuzumab and wherein the neoplastic disease is a HER2-positive cancer, in particular HER2-positive breast, gastric, ovarian or lung cancer;
   wherein the antibody-payload conjugate comprises Polatuzumab and wherein the neoplastic disease is a B-cell associated cancer; preferably, wherein the B-cell associated cancer is non-Hodgkin lymphoma, in particular wherein the B-cell associated cancer is diffuse large B-cell lymphoma; or
   wherein the antibody-payload conjugate comprises Enfortumab or an Enfortumab variant and wherein the neoplastic disease is a Nectin-4 positive cancer, in particular Nectin-4 positive pancreatic cancer, lung cancer, bladder cancer or breast cancer.
P15. An antibody-drug conjugate (ADC) having the formula A-L, wherein A is an antibody or an antibody fragment and wherein L is a linker, said linker comprising:
   as a first payload a camptothecin cytotoxic molecule which is cell-permeable; and
   as a second payload a camptothecin cytotoxic molecule which is not cell-permeable;
   wherein the cytotoxic molecule of the first and second payload is an exatecan; wherein the second payload has a glycine residue linked to said camptothecin cytotoxic molecule of the second payload;
   wherein said ADC consists of two first payloads and two second payloads (drug-to-antibody ratio of 4 “DAR4”); and
   wherein said antibody is an IgG, preferably, an IgG1 antibody.
P16. The ADC according to embodiment 15, wherein the antibody is Trastuzumab.
1. An antibody-drug conjugate (ADC) having the formula A-L, wherein A is an antibody or an antibody fragment and wherein L is a linker, said linker comprising:
   as a first payload a topoisomerase I inhibitor which is cell-permeable, preferably a camptothecin cytotoxic molecule which is cell-permeable; and
   as a second payload a topoisomerase I inhibitor which is not cell-permeable, preferably a camptothecin cytotoxic molecule which is not cell-permeable.
2. The ADC according to embodiment 1, wherein the camptothecin is an exatecan or a deruxtecan.
3. The ADC according to embodiment 1 or 2, wherein the second payload has been modified to reduce its cell permeability.
4. The ADC according to any one of embodiments 1 to 3, wherein the second payload has a glycine residue linked to said topoisomerase I inhibitor of the second payload.
5. The ADC according to any one of embodiments 1 to 4, wherein the linker is a peptide linker.
6. The ADC according to embodiment 5, wherein the first payload and/or second payload are linked to the N- or C-terminus of the peptide linker or to a side-chain of an amino acid residue comprised in the peptide linker; preferably
   wherein the first payload is linked to the N-terminus of the peptide linker and wherein the second payload is linked to the C-terminus of the peptide linker, or vice versa.
7. The ADC according to any of of embodiments 1 to 6, wherein the linker is conjugated to the antibody via an isopeptide bond formed between a glutamine residue comprised in the antibody and a primary amine comprised in the linker, preferably wherein the primary amine is comprised in a lysine residue, a lysine mimetic or a lysine derivative comprised in a peptide linker or wherein the primary amine is comprised in an amino acid residue having the structure NH₂—(CH₂)₁₋₁₀—COOH comprised in a peptide linker.
8. The ADC according to any one of embodiments 1 to 7, wherein the linker further comprises at least one positively charged amino acid residue, preferably wherein the at least one positively charged amino acid residue is selected from arginine and/or histidine.
9. The ADC according to any one of embodiments 1 to 8, wherein the linker consists of or comprises the following structure:

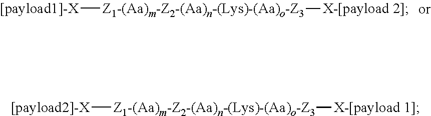

wherein
[payload 1] is said first payload;
[payload 2] is said second payload;
(Aa) is any amino acid residue;
m, n and o may be integers ranging from 0 to 10, preferably 0 to 6, more preferably 0 to 4;
(Lys) is a lysine residue, a lysine mimetic or a lysine derivative;
Z₁₋₃ is either absent or a spacer comprising an alkyl or a heteroalkyl group, preferably wherein the spacer comprises (C_{H}2)₂; and
X is either absent or a self-immolative group, preferably PABC.
10. The ADC according to embodiment 9, wherein (Aa)ₘ+(Aa)ₙ+(Aa)ₒ is >0, preferably wherein (Aa)ₙ+(Aa)ₒ is >0.
11. The ADC according to embodiment 9 or 10, wherein Z₂ is a dicarboxylic acid linking the N-terminal end of (Aa)ₘ to the N-terminal end of (Aa)ₙ or (Lys); and wherein one payload is directly or indirectly linked to the C-terminal end of (Aa)ₘ and the other payload is directly or indirectly linked to the C-terminal end of (Lys) or (Aa)ₒ, or vice versa.
12. The ADC according to any one of embodiments 9 to 11, wherein the linker consists of or comprises the following structure:

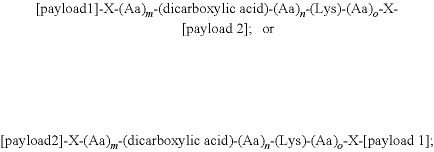

wherein
[payload 1] is said first payload;
[payload 2] is said second payload;
(Aa) is any amino acid residue;
m is an integer ranging from 1 to 10, preferably 1 to 6, more preferably 1 to 4;
n and o may be integers ranging from 0 to 10, preferably 0 to 6, more preferably 0 to 4, wherein n+o is >0;
(Lys) is a lysine residue, a lysine mimetic or a lysine derivative;
(dicarboxylic acid) is dicarboxylic acid linking the N-terminal end of (Aa)ₘ to the N-terminal end of (Aa)ₙ or (Lys); and
X is either absent or a self-immolative group, preferably PABC.
13. The ADC according to any one of embodiments 9 to 12, wherein (Aa)ₙ-(Lys)-(Aa)ₒ comprises the sequence motif Arg-Lys (RK) or His-Lys (HK) (in N->C direction).
14. The ADC according to any one of embodiments 9 to 13, wherein (Aa)ₙ-(Lys)-(Aa)ₒ is or comprises RK or RKAA (in N->C direction).
15. The ADC according to any one of embodiments 1 to 14, wherein the linker comprises or consists of the following structure:

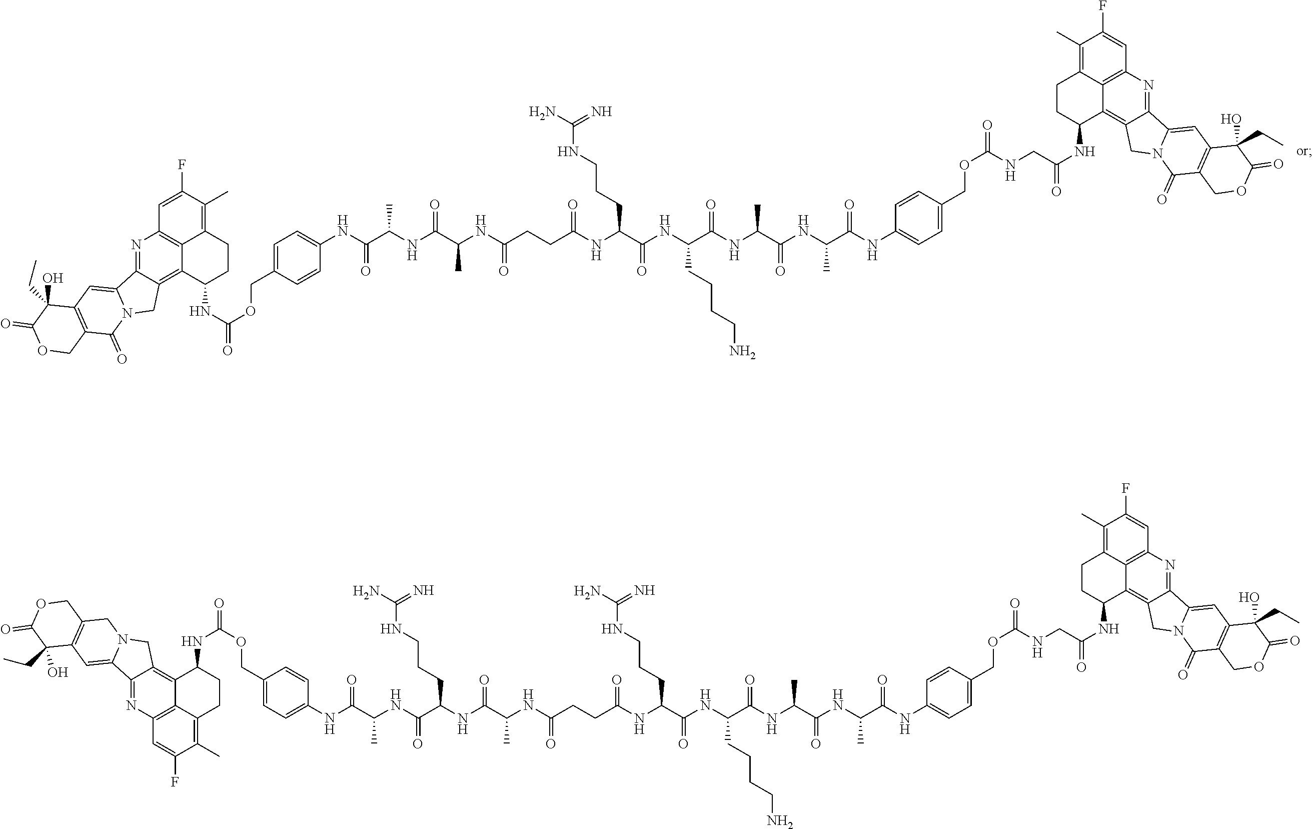

16. The ADC according to any one of embodiments 1 to 15, wherein said ADC comprises more than one first payloads and/or more than one second payloads.
17. The ADC according to any one of embodiments 1 to 16, wherein said ADC consists of two first payloads and two second payloads (drug-to-antibody ratio of 4 “DAR4”).
18. The ADC according to any one of embodiments 1 to 16, wherein said linker further comprises a third payload, preferably a toxin or a cytotoxin, more preferably, an auristatin, and even more preferably, an MMAE (Monomethyl auristatin E).
19. The ADC according to embodiment 18, wherein the linker is a peptide linker and wherein the third payload is linked to the N- or C-terminus of the peptide linker or wherein the third payload is linked to a side chain of an amino acid residue comprised in the peptide linker.
20. The ADC according to embodiment 19, wherein two payloads are linked to the same functional group of the peptide linker, preferably via a chemical linker comprising a disubstituted amine.
21. The ADC according to embodiment 20, wherein the linker consists of or comprises the structure:

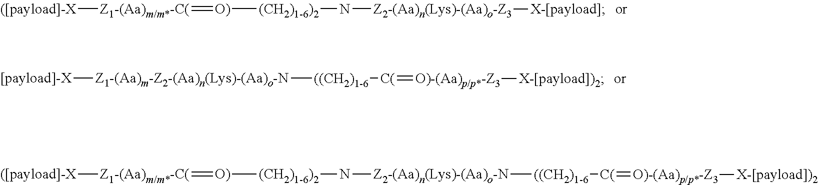

wherein
[payload] is each independently a payload selected from the first, second and third payload, wherein the linker comprises all three payloads;
(Aa) is any amino acid residue;
m, m* n, o, p and p* may be integers ranging from 0 to 10, preferably 0 to 6, more preferably 0 to 4;
(Lys) is a lysine residue, a lysine mimetic or a lysine derivative;
Z₁₋₃ is either absent or a spacer comprising an alkyl or a heteroalkyl group, preferably wherein the spacer comprises (CH₂)₂, even more preferably wherein Z₂ is a dicarboxylic acid linker; and
X is either absent or a self-immolative group, preferably PABC.
22. The ADC according to embodiment 21, wherein (Aa)ₙ+(Aa)ₒ is >0.
23. The ADC according to any one of embodiments 20 to 22, wherein the linker consists of or comprises the structure:

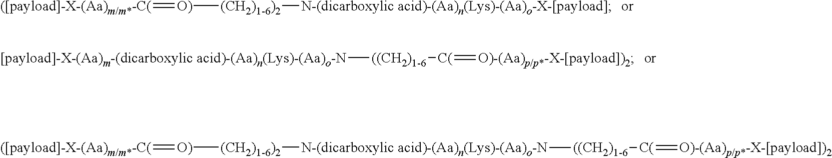

wherein
[payload] is each independently a payload selected from the first, second and third payload, wherein the linker comprises all three payloads;
(Aa) is any amino acid residue;
m, m*, p and p* are integers ranging from 1 to 10, preferably 1 to 6, more preferably 1 to 4;
n and o may be integers ranging from 0 to 10, preferably 0 to 6, more preferably 0 to 4, wherein n+o is >0;
(Lys) is a lysine residue, a lysine mimetic or a lysine derivative;
(dicarboxylic acid) is dicarboxylic acid linking the N-terminal end of (Aa)ₘ or the disubstituted amine (N) to the N-terminal end of (Aa)ₙ or (Lys); and
X is either absent or a self-immolative group, preferably PABC.
24. The ADC according to any one of embodiments 21 to 23, wherein (Aa)ₙ-(Lys)-(Aa)ₒ comprises the sequence motif Arg-Lys (RK) or His-Lys (HK) (in N->C direction).
25. The ADC according to any one of embodiments 21 to 24, wherein (Aa)ₙ-(Lys)-(Aa)ₒ is or comprises RK or RKAA (in N->C direction).
26. The ADC according to any one of embodiments 18 to 25, wherein the linker comprises or consists of the following structure:

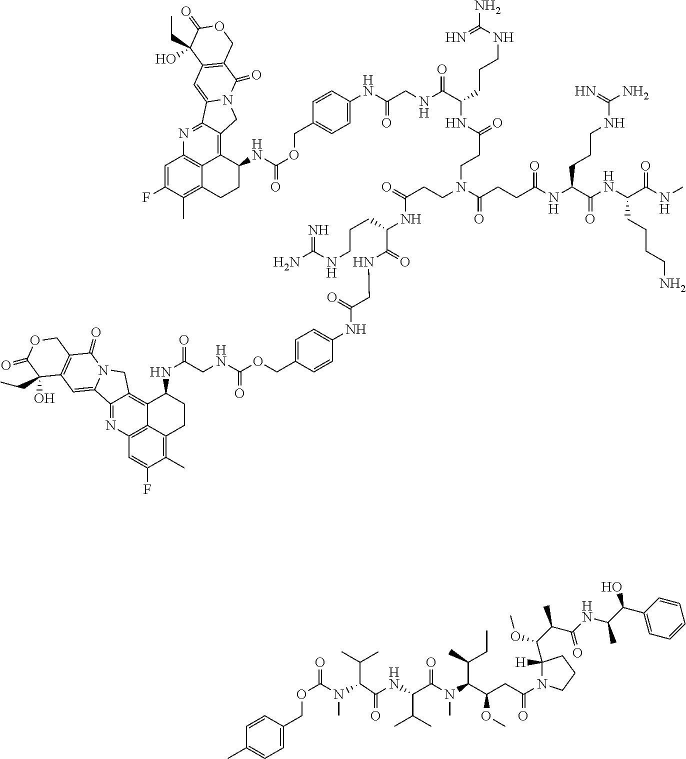

27. The ADC according to any one of embodiments 1 to 26, wherein the antibody is an IgG antibody, preferably an IgG1 or IgG4 antibody.
28. The ADC according to any one of embodiments 7 to 27, wherein the glutamine residue is residue Q295 (EU numbering) of the C_{H}2 domain of an IgG antibody.
29. The ADC according to any one of embodiments 1 to 28, wherein the antibody is a glycosylated antibody, preferably wherein the antibody is an IgG antibody glycosylated at residue N297 (EU numbering).
30. The ADC according to any one of embodiments 1 to 29,
   wherein the antibody is selected from the group consisting of: Trastuzumab, Brentuximab, Gemtuzumab, Inotuzumab, Avelumab, Cetuximab, Rituximab, Daratumumab, Pertuzumab, Vedolizumab, Ocrelizumab, Tocilizumab, Ustekinumab, Golimumab, Obinutuzumab, Sacituzumab, Belantamab, Polatuzumab, Enfortumab, Endrecolomab, Gemtuzumab,Loncastuximab, Mecbotamab, Adecatumumab, D93, Gatipotuzumab, Labetuzumab, Tusamitamab, Upifitamab, Lifastuzumab, Mirvetuximab, Sofituzumab, Anetumab, Tisotumab, Cofituzumab, Praluzatamab, Ladriatuzumab, Belantamab, Patritumab, Cetuximab, Nimotuzumab, Matuzumab, Portuzumab, Citatuzumab, Tucotuzumab and Endrecolomab; and/or
   wherein the antibody specifically binds to an antigen selected from the group consisting of: CD30, Her2/neuCD33, CD22, PD-L1, EGFR, CD20, CD38, HER2, Integrin α4β7, CD20, IL-6-R, IL-12, IL-23, TNFα, CD20, Trop-2, BCMA, CD79b, Nectin-4, EpCAM, CD33, CD19, AXL, dn-collagen, TA-MUC1, carcinoembryonic cell adhesion molecule 5, CEACAM5, NaPi2b, FRα, MUC16, mesothelin, TF, CD166, LIV-1, ERBB3, EGFR, and TACSTD1, preferably, CD30, Her2/neu, CD22, CD79b, Nectin-4, Trop-2 and BCMA, more preferably, CD79b, Her2/neu, and Nectin-4.
31. A pharmaceutical composition comprising the ADC according to any one of embodiments 1 to 30 and at least one pharmaceutically acceptable ingredient.
32. The ADC according to any one of embodiments 1 to 30 or the pharmaceutical composition of claim 28 for use in a method of treating a patient suffering from, being at risk of developing, and/or being diagnosed for a neoplastic disease, in particular wherein the neoplastic disaease is cancer.
33. The ADC according to any one of embodiments 1 to 30 and 32 or the pharmaceutical composition of embodiment 31 or 32,
   wherein the antibody-payload conjugate comprises Trastuzumab and wherein the neoplastic disease is a HER2-positive cancer, in particular HER2-positive breast, gastric, ovarian or lung cancer;
   wherein the antibody-payload conjugate comprises Polatuzumab and wherein the neoplastic disease is a B-cell associated cancer; preferably, wherein the B-cell associated cancer is non-Hodgkin lymphoma, in particular wherein the B-cell associated cancer is diffuse large B-cell lymphoma; or
   wherein the antibody-payload conjugate comprises Enfortumab or an Enfortumab variant and wherein the neoplastic disease is a Nectin-4 positive cancer, in particular Nectin-4 positive pancreatic cancer, lung cancer, bladder cancer or breast cancer.
34. The ADC according to any one of embodiments 1 to 30 and 32 or the pharmaceutical composition of embodiment 31 or 32,
   wherein the antibody-payload conjugate comprises an antibody targeting NaPi2b, preferably wherein the antibody targeting NaPi2b is Upifitamab with a heavy chain as set forth in SEQ ID NO:98 and a light chain as set forth in SEQ ID NO:99, or with a heavy chain as set forth in SEQ ID NO:100 and a light chain as set forth in SEQ ID NO:101, and wherein the neoplastic disease is a NaPi2b positive cancer, in particular NaPi2b positive lung or ovarian cancer,
   preferably wherein the ADC comprises a linker having the structure:

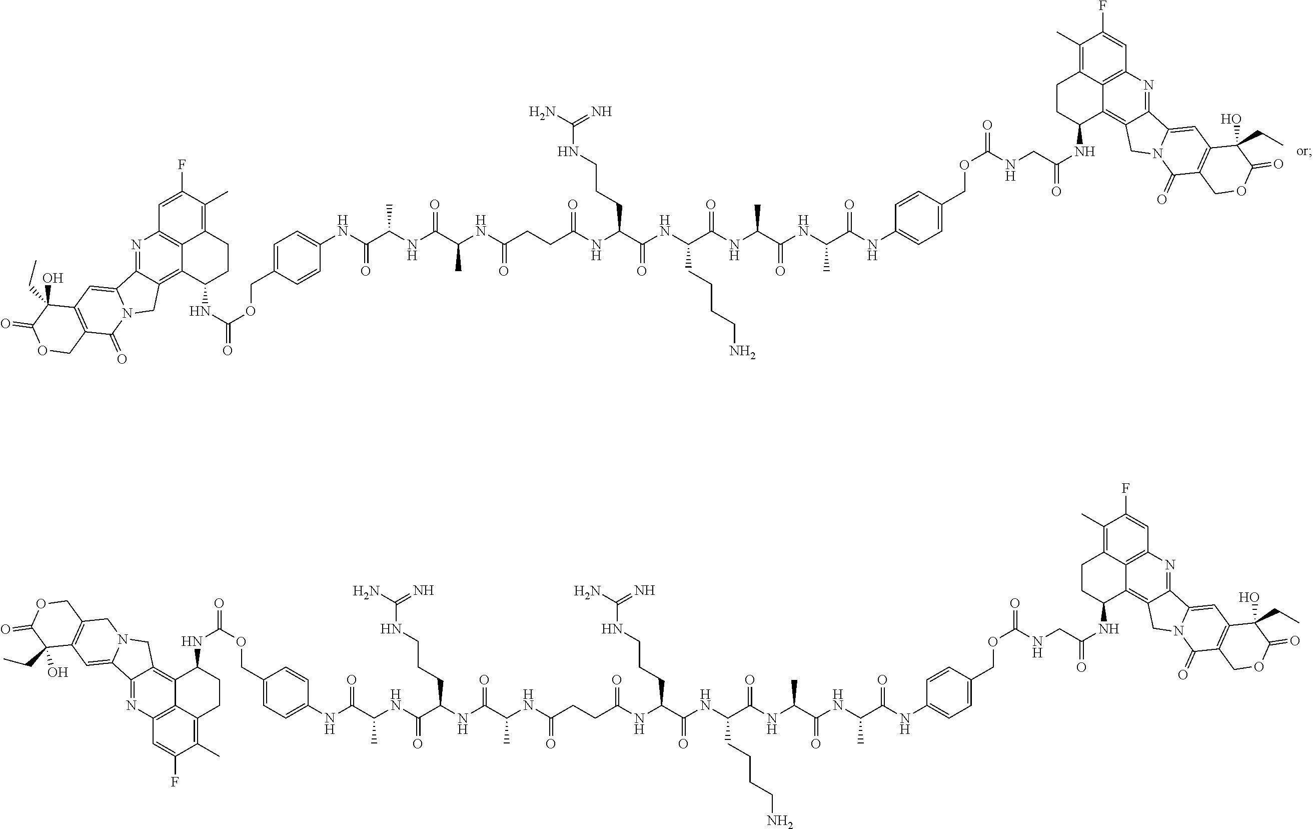

35. The ADC according to any one of embodiments 1 to 30 and 32 or the pharmaceutical composition of embodiment 31 or 32,
   wherein the antibody-payload conjugate comprises an antibody targeting Nectin-4, preferably wherein the antibody targeting Nectin-4 is m290 with a heavy chain as set forth in SEQ ID NO:96 and a light chain as set forth in SEQ ID NO:97, or Enfortumab with a heavy chain as set forth in SEQ ID NO:75 or 94 and a light chain as set forth in SEQ ID NO:95, 76 or 77, and wherein the neoplastic disease is a Nectin-4 positive cancer, in particular Nectin-4 positive pancreatic cancer, lung cancer, bladder cancer or breast cancer,
   preferably wherein the ADC comprises a linker having the structure:

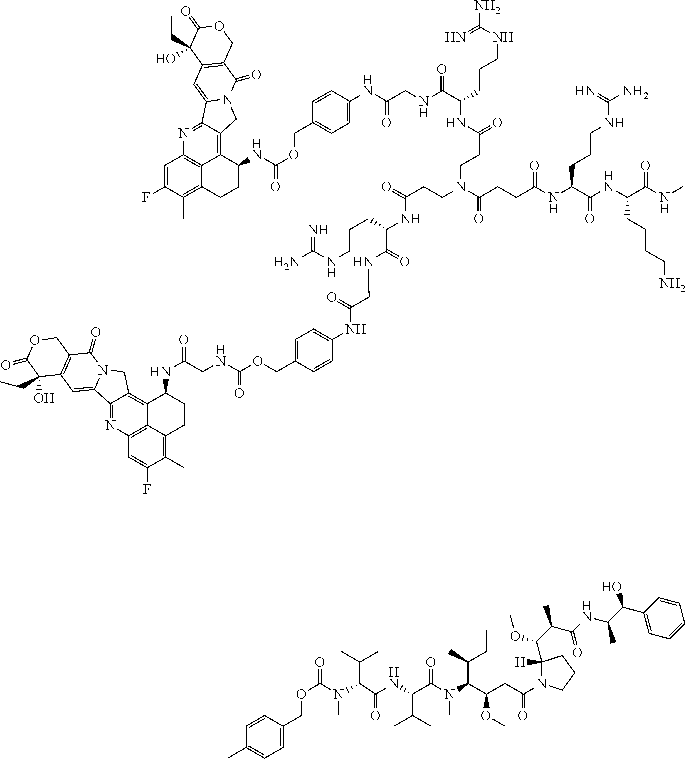

36. A linker comprising a first and second payload, wherein the first payload is a topoisomerase I inhibitor which is cell-permeable, preferably a camptothecin cytotoxic molecule which is cell-permeable; and
   wherein the second payload a topoisomerase I inhibitor which is not cell-permeable, preferably a camptothecin cytotoxic molecule which is not cell-permeable, preferably wherein the linker is for conjugation to an antibody
37. The linker according to embodiment 36, wherein the camptothecin is an exatecan or a deruxtecan.
38. The linker according to embodiment 36 or 37, wherein the second payload has been modified to reduce its cell permeability.
39. The linker according to any one of embodiments 36 to 38, wherein the second payload has a glycine residue linked to said topoisomerase I inhibitor of the second payload.
40. The linker according to any one of embodiments 36 to 39, wherein the linker is a peptide linker.
41. The linker according to embodiment 40, wherein the first payload and/or second payload are linked to the N- or C-terminus of the peptide linker or to a side-chain of an amino acid residue comprised in the peptide linker; preferably
   wherein the first payload is linked to the N-terminus of the peptide linker and wherein the second payload is linked to the C-terminus of the peptide linker, or vice versa.
42. The linker according to any of of embodiment 36 to 41, wherein the linker comprises a primary amine for conjugation to an antibody, preferably wherein the primary amine is comprised in a lysine residue, a lysine mimetic or a lysine derivative; or
   wherein the primary amine is comprised in an amino acid residue having the structure NH₂—(CH₂)₁₋₁₀—COOH; preferably wherein the amino acid residues are comprised in a peptide linker.
43. The linker according to any one of embodiments 36 to 42, wherein the linker further comprises at least one positively charged amino acid residue, preferably wherein the at least one positively charged amino acid residue is selected from arginine and/or histidine.
44. The linker according to any one of embodiments 36 to 43, wherein the linker consists of or comprises the following structure:

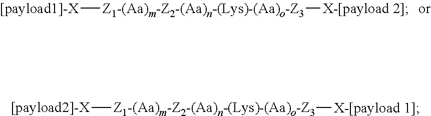

wherein
[payload 1] is said first payload;
[payload 2] is said second payload;
(Aa) is any amino acid residue;
m, n and o may be integers ranging from 0 to 10, preferably 0 to 6, more preferably 0 to4;
(Lys) is a lysine residue, a lysine mimetic or a lysine derivative;
Z₁₋₃ is either absent or a spacer comprising an alkyl or a heteroalkyl group, preferably wherein the spacer comprises (C_{H}2)₂; and
X is either absent or a self-immolative group, preferably PABC.
45. The linker according to embodiment 44, wherein (Aa)ₘ+(Aa)ₙ+(Aa)ₒ is >0, preferably wherein (Aa)ₙ+(Aa)ₒ is >0.
46. The linker according to embodiment 44 or 45, wherein Z₂ is a dicarboxylic acid linking the N-terminal end of (Aa)ₘ to the N-terminal end of (Aa)ₙ or (Lys); and wherein one payload is directly or indirectly linked to the C-terminal end of (Aa)ₘ and the other payload is directly or indirectly linked to the C-terminal end of (Lys) or (Aa)ₒ, or vice versa.
47. The linker according to any one of embodiments 44 to 46, wherein the linker consists of or comprises the following structure:
   [payload1]-X-(Aa)ₘ-(dicarboxylic acid)-(Aa)ₙ(Lys)-(Aa)ₒ-X-[payload 2]; or
   [payload2]-X-(Aa)ₘ-(dicarboxylic acid)-(Aa)ₙ(Lys)-(Aa)ₒ-X-[payload 1];
   wherein
   [payload 1] is said first payload;
   [payload 2] is said second payload;
   (Aa) is any amino acid residue;
   m is an integer ranging from 1 to 10, preferably 1 to 6, more preferably 1 to 4;
   n and o may be integers ranging from 0 to 10, preferably 0 to 6, more preferably 0 to 4, wherein n+o is >0;
   (Lys) is a lysine residue, a lysine mimetic or a lysine derivative;
   (dicarboxylic acid) is dicarboxylic acid linking the N-terminal end of (Aa)ₘ to the N-terminal end of (Aa)ₙ or (Lys); and
   X is either absent or a self-immolative group, preferably PABC.
48. The linker according to any one of embodiments 44 to 47, wherein (Aa)ₙ-(Lys)-(Aa)ₒ comprises the sequence motif Arg-Lys (RK) or His-Lys (HK) (in N->C direction).
49. The linker according to any one of embodiments 44 to 48, wherein (Aa)ₙ-(Lys)-(Aa)ₒ is or comprises RK or RKAA (in N->C direction).
50. The linker according to any one of embodiments 36 to 49, wherein the linker comprises or consists of the following structure:

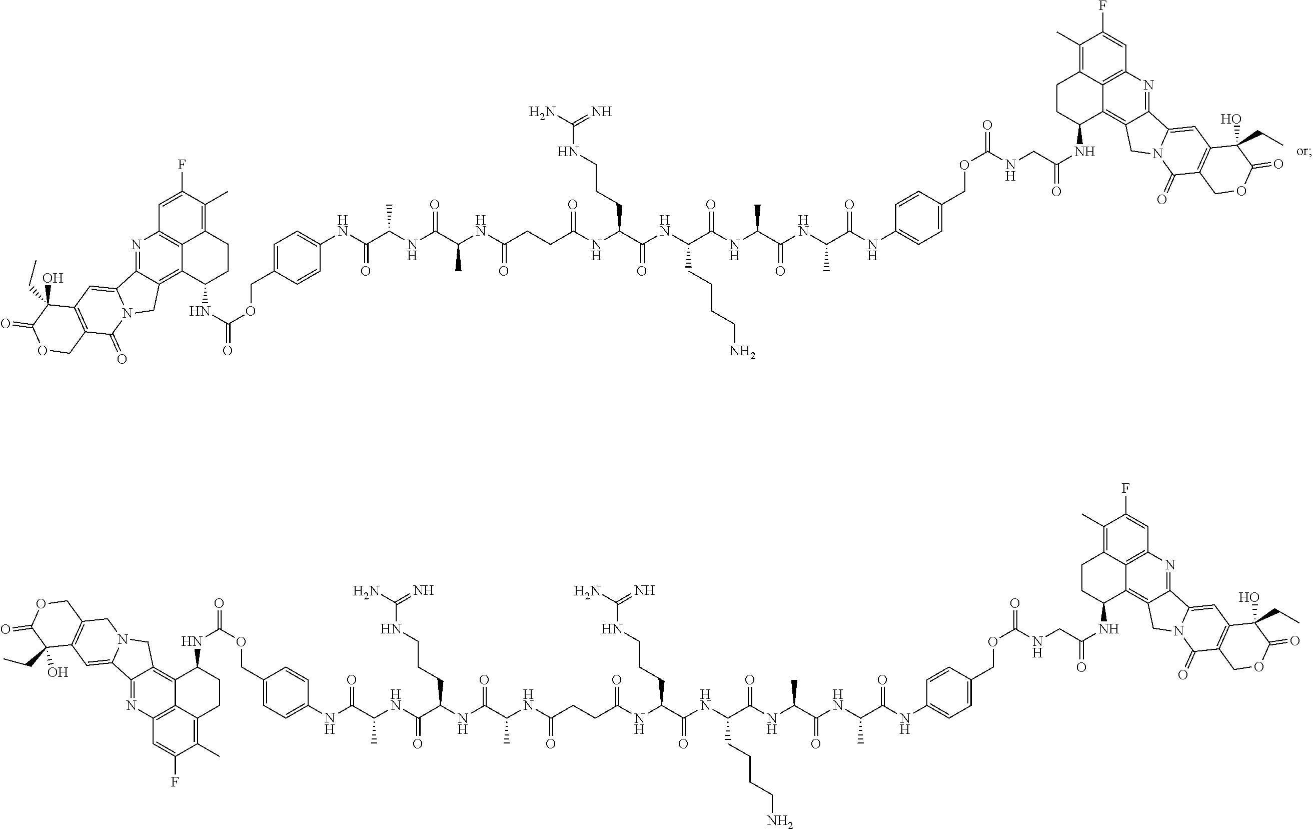

51. The linker according to any one of embodiments 36 to 50, wherein said linker further comprises a third payload, preferably a toxin or a cytotoxin, more preferably, an auristatin, and even more preferably, an MMAE (Monomethyl auristatin E).
52. The linker according to embodiment 51, wherein the linker is a peptide linker and wherein the third payload is linked to the N- or C-terminus of the peptide linker or wherein the third payload is linked to a side chain of an amino acid residue comprised in the peptide linker.
53. The linker according to embodiment 52, wherein two payloads are linked to the same functional group of the peptide linker, preferably via a chemical linker comprising a disubstituted amine.
54. The linker according to embodiment 53, wherein the linker consists of or comprises the structure:

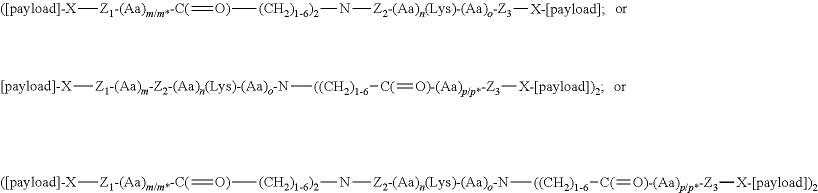

wherein
[payload] is each independently a payload selected from the first, second and third payload, wherein the linker comprises all three payloads;
(Aa) is any amino acid residue;
m, m*, n, o, p and p* may be integers ranging from 0 to 10, preferably 0 to 6, more preferably 0 to 4;
(Lys) is a lysine residue, a lysine mimetic or a lysine derivative;
Z₁₋₃ is either absent or a spacer comprising an alkyl or a heteroalkyl group, preferably wherein the spacer comprises (CH₂)₂, even more preferably wherein Z₂ is a dicarboxylic acid linker; and
X is either absent or a self-immolative group, preferably PABC.
55. The linker according to embodiment 54, wherein (Aa)ₙ+(Aa)ₒ is >0.
56. The linker according to any one of embodiments 53 to 55, wherein the linker consists of or comprises the structure:

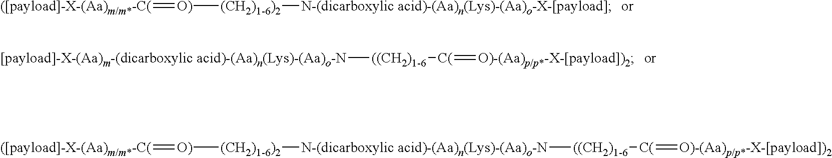

wherein
[payload] is each independently a payload selected from the first, second and third payload, wherein the linker comprises all three payloads;
(Aa) is any amino acid residue;
m, m*, p and p* are integers ranging from 1 to 10, preferably 1 to 6, more preferably 1 to 4;
n and o may be integers ranging from 0 to 10, preferably 0 to 6, more preferably 0 to 4, wherein n+o is >0;
(Lys) is a lysine residue, a lysine mimetic or a lysine derivative; (dicarboxylic acid) is dicarboxylic acid linking the N-terminal end of (Aa)ₘ or the disubstituted amine (N) to the N-terminal end of (Aa)ₙ or (Lys); and
X is either absent or a self-immolative group, preferably PABC.
57. The linker according to any one of embodiments 54 to 56, wherein (Aa)ₙ-(Lys)-(Aa)ₒ comprises the sequence motif Arg-Lys (RK) or His-Lys (HK) (in N->C direction).
58. The linker according to any one of embodiments 54 to 57, wherein (Aa)ₙ-(Lys)-(Aa)ₒ is or comprises RK or RKAA (in N->C direction).
59. The linker according to any one of embodiments 52 to 58, wherein the linker comprises or consists of the following structure:

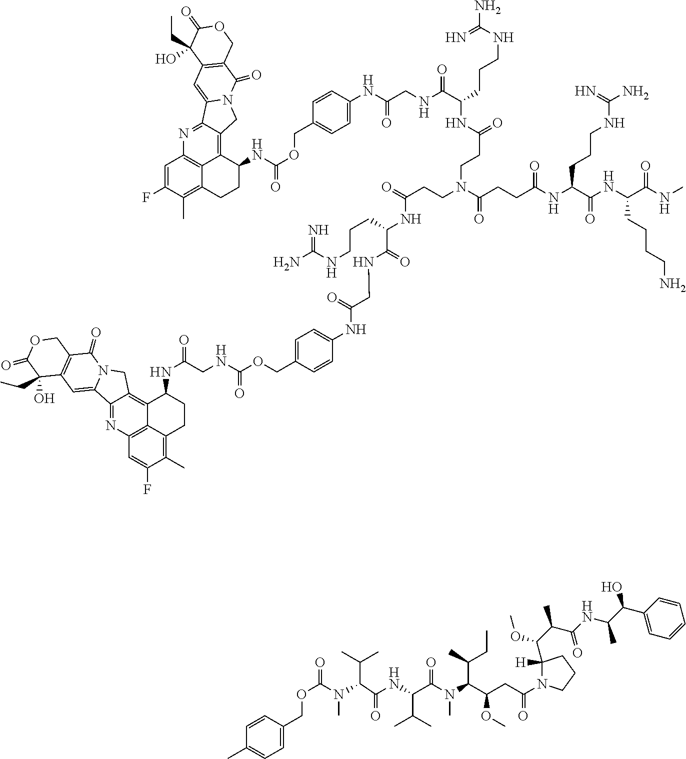

60. A method for the preparation of an antibody-drug conjugate comprising a step of conjugating a peptide linker according to any of embodiments 36 to 59 to an antibody.
61. A method for the conjugation of a peptide linker according to any one of embodiments 40 to 59 using a transglutaminase (TG), the method comprising a) mixing the antibody, the peptide linker and a transglutaminase (TG) within a fluid, thereby conjugating the linker-payload to the antibody in one step under the catalyzing effect of the TG, and b) extracting the conjugate obtained in step a) from the fluid.
62. The method according to embodiment 61, wherein the peptide linker is conjugated to a glutamine residue comprised in the antibody via a primary amine comprised in an amino acid residue of the peptide linker.
63. The method according to embodiment 61 or 62, wherein the peptide linker is conjugated to a glutamine residue comprised in an Fc domain of an IgG antibody.
64. The method according to embodiment 62 or 63, wherein the glutamine residue to which the peptide linker is conjugated is glutamine residue Q295 (EU numbering) of the C_{H}2 domain of an IgG antibody.
65. The method according to any one of embodiments 61 to 64, wherein the antibody is a glycosylated IgG antibody.
66. The method according to embodiment 65, wherein the IgG antibody is glycosylated at residue N297 (EU numbering) of the C_{H}2 domain.
67. The method according to any one of embodiments 61 to 66, wherein the antibody is selected from the group consisting of: m290, Trastuzumab, Brentuximab, Gemtuzumab, Inotuzumab, Avelumab, Cetuximab, Rituximab, Daratumumab, Pertuzumab, Vedolizumab, Ocrelizumab, Tocilizumab, Ustekinumab, Golimumab, Obinutuzumab, Sacituzumab, Belantamab, Polatuzumab, Enfortumab, Endrecolomab, Gemtuzumab,Loncastuximab, Mecbotamab, Adecatumumab, D93, Gatipotuzumab, Labetuzumab, Tusamitamab, Upifitamab, Lifastuzumab, Mirvetuximab, Sofituzumab, Anetumab, Tisotumab, Cofituzumab, Praluzatamab, Ladriatuzumab, Belantamab, Patritumab, Cetuximab, Nimotuzumab, Matuzumab, Portuzumab, Citatuzumab, Tucotuzumab and Endrecolomab; and/or
   wherein the antibody specifically binds to an antigen selected from the group consisting of: CD30, Her2/neuCD33, CD22, PD-L1, EGFR, CD20, CD38, HER2, Integrin α4β7, CD20, IL-6-R, IL-12, IL-23, TNFα, CD20, Trop-2, BCMA, CD79b, Nectin-4, EpCAM, CD33, CD19, AXL, dn-collagen, TA-MUC1, carcinoembryonic cell adhesion molecule 5, CEACAM5, NaPi2b, FRα, MUC16, mesothelin, TF, CD166, LIV-1, ERBB3, EGFR, and TACSTD1, preferably, CD30, Her2/neu, CD22, CD79b, Nectin-4, Trop-2 and BCMA, more preferably, CD79b, Her2/neu, and Nectin-4.
68. The method according to any one of embodiments 61 to 67, wherein the peptide linker is suitable for conjugation to a glycosylated antibody with a conjugation efficiency of at least 20%, 30%, 40%, 50%, 60%, 70%, 75%, 80%, 85%, 90% or 95%.
69. The method according to any one of embodiments 61 to 68, wherein the transglutaminase is a microbial transglutaminase (MTG), preferably wherein the microbial transglutaminase is derived from a Streptomyces species, in particular Streptomyces mobaraensis.
70. An antibody-drug conjugate (ADC) having the formula A-L, wherein A is an antibody or an antibody fragment and wherein L is a linker, said linker comprising:
   as a first payload a camptothecin cytotoxic molecule which is cell-permeable; and
   as a second payload a camptothecin cytotoxic molecule which is not cell-permeable;
   wherein the cytotoxic molecule of the first and second payload is an exatecan;
   wherein the second payload has a glycine residue linked to said camptothecin cytotoxic molecule of the second payload;
   wherein said ADC consists of two first payloads and two second payloads (drug-to-antibody ratio of 4 “DAR4”); and
   wherein said antibody is an IgG, preferably, an IgG1 antibody.
71. The ADC according to embodiment 70, wherein the antibody is Trastuzumab or Upifitamab.
72. An antibody-drug conjugate (ADC) having the formula A-L, wherein A is an antibody or an antibody fragment and wherein L is a linker, said linker comprising:
   as a first payload a camptothecin cytotoxic molecule which is cell-permeable;
   as a second payload a camptothecin cytotoxic molecule which is not cell-permeable;
   wherein the cytotoxic molecule of the first and second payload is an exatecan;
   wherein the second payload has a glycine residue linked to said camptothecin cytotoxic molecule of the second payload; and
   as a third payload an auristatin, preferably an MMAE;
   wherein said ADC consists of two first payloads, two second payloads and two third payloads (drug-to-antibody ratio of 6 “DAR6”); and
   wherein said antibody is an IgG, preferably, an IgG1 antibody.
73. The ADC according to embodiment 15, wherein the antibody is m290.

### BRIEF DESCRIPTION OF THE DRAWINGS

FIG. 1A: Cytotoxic activity of T-Exa-PABC-AA-C2-RKAA-PABC-G-Exa on HER2-overexpressing SK-BR3 breast cancer cells.

FIG. 1B: Cytotoxic activity of T-Exa-PABC-ARA-C2-RKAA-PABC-G-Exa on HER2-overexpressing SK-BR3 breast cancer cells.

FIG. 2: Cell counts of viable cells after incubation of the indicated ADCs.

FIG. 3A: T-Exa-PABC-AA-C2-RKAA-PABC-G-Exa shows superior and long-lasting anti-tumor activity in vivo compared to anti-HER2-ADC DS-8201a.

FIG. 3B: The ADC of the present invention T-Exa-PABC-ARA-C2-RKAA-PABC-G-Exa confirms highly efficient anti-tumor efficacy and shows superior and long-lasting anti-tumor activity in vivo compared to anti-HER2-ADC DS-8201a.

FIG. 4: Linker structure: Exa-PABC-AA-C2-RKAA-PABC-G-Exa (upper) and Exa-PABC-ARA-C2-RKAA-PABC-G-Exa (lower).

FIG. 5: Schematic depiction of a DAR4 (2+2) ADC of this invention with two different Exatecan.

FIG. 6: Schematic depiction of T-Exa-PABC-AA-C2-RKAA-PABC-G-Exa.

FIG. 7: Schematic depiction of T-Exa-PABC-ARA-C2-RKAA-PABC-G-Exa.

FIG. 8: Linker structure with three payloads: (Exa-PABC-GR/Exa-G-PABC-GR)-CEA-C2-RK-PABC-MMAE.

FIG. 9: Anti-tumoral effects of ARC-121 vs FDA approved ADC Padcev® FIG. 10: Anti-tumoral effects of ARC-121 and FDA approved ADCs Trodelvy® and Padcev®

### EXAMPLES

### 1. General Material and Methods for the Production of ADC

Enhertu® (DS-8201a or “fam-trastuzumab deruxtecan-nxki”) and Herceptin® (trastuzumab) were bought at the pharmacy.

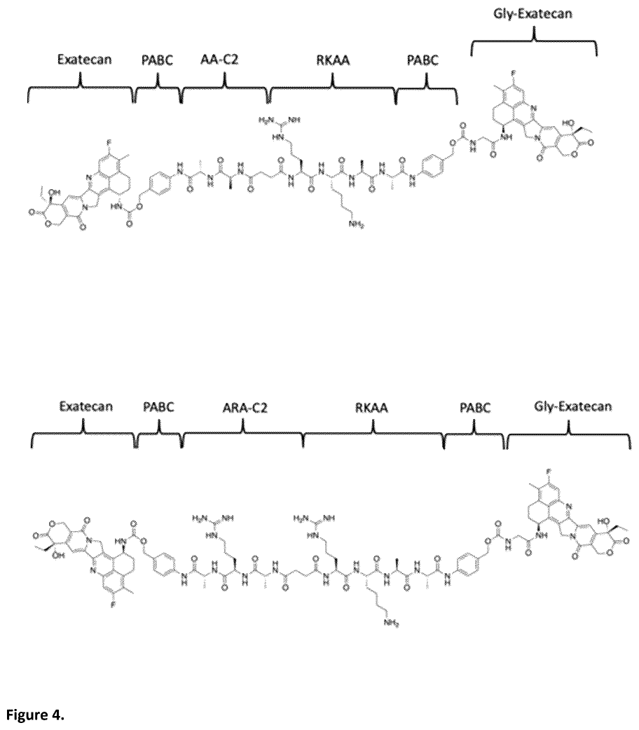

Linker-payloads Exa-PABC-AA-C2-RKAA-PABC-G-Exa and Exa-PABC-ARA-C2-RKAA-PABC-G-Exa (see FIG. 4 for chemical structure) were custom synthesized by WuXi.

As it can be seen in FIG. 4, they both contain an exatecan (“Exa”) and a glycine-exatecan (“Gly-Exa”) topoisomerase I inhibitor.

ADCs of the invention “T-Exa-PABC-AA-C2-RKAA-PABC-G-Exa” and “T-Exa-PABC-ARA-C2-RKAA-PABC-G-Exa”:

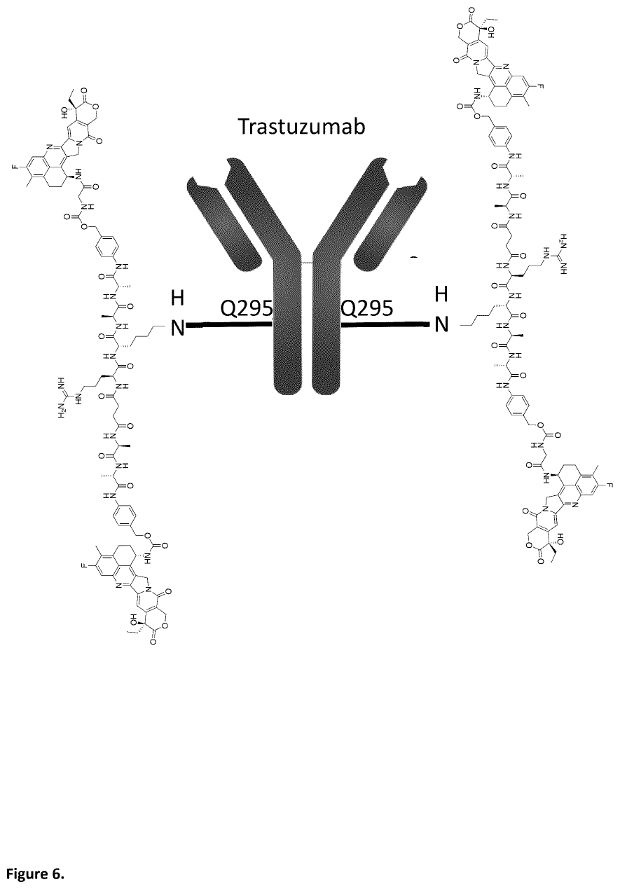

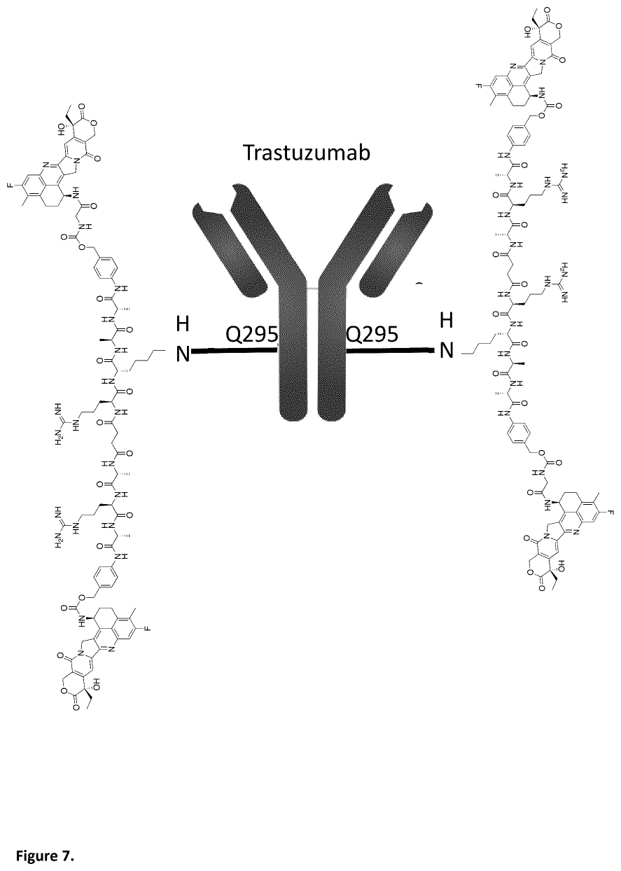

In order to generate the ADCs of the invention “T-Exa-PABC-AA-C2-RKAA-PABC-G-Exa” (FIG. 6) and “T-Exa-PABC-ARA-C2-RKAA-PABC-G-Exa” (FIG. 7), an enzymatic method using linkers with a lysine residue as described by Spycher et al. (WO 2019/057772 and WO 2020/188061) was used.

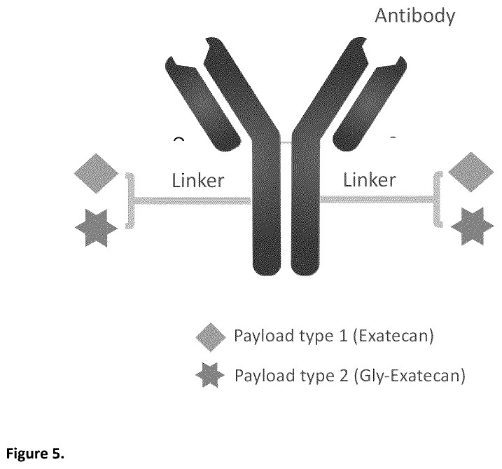

Briefly, conjugation reactions were performed by mixing 5 mg/ml of native, glycosylated monoclonal antibody trastuzumab (rebuffered in reaction buffer, see below), microbial transglutaminase (MTG, Zedira) at a concentration of 5-10 U/mg, and 5-20 molar equivalents of the indicated linker-payload, in BisTris pH 6.0-6.8 for 24 hours at 37° C. in a rotating thermomixer. In this way, linker-payloads of the invention were conjugated to glutamine 295 of each antibody (see FIG. 5) resulting in a DAR4 (2+2).

Conjugation efficiency was assessed by LC-MS, under TCEP reduced conditions (calculations see below). Reduction of samples was achieved by incubation of the samples for 15 min at 37° C. in 50 mM TCEP (final) and 50 mM Tris buffer.

Calculation of conjugation efficiency by LCMS: after reduction, samples were analyzed on a Compact QTOF spectrometer (Bruker) coupled to an 1260 Infinity II LC (Agilent) equipped with an ACQUITY Premier Protein BEH C4 Column. Conjugation efficiency (CE) was calculated from deconvoluted spectra and presented in %. Intensities resulting from both glycoforms (G1F and GOF) were taken into account for the calculation, according to the formula:

$$CE \% = \frac{\sum ( {( Int ( G 0 F + G 1 F ) )}_{cj} )}{\sum {( Int ( G 0 F + G 1 F ) )}_{cj , ncj}}$$

With cj=conjugated and ncj=non-conjugated

The conjugation efficiency for the ADCs “T-Exa-PABC-AA-C2-RKAA-PABC-G-Exa” and “T-Exa-PABC-ARA-C2-RKAA-PABC-G-Exa” was at least 95%.

### 2. Example 1: Antibody-Drug Conjugates Using Two Different Types of Topoisomerase I Inhibitors Show Potent Anti-Tumor Effects In Vitro

In order to assess whether the ADCs of the invention are able to kill HER2 positive tumor cells, an in vitro cytotoxicity assay was performed.

### Method

For a cell cytotoxicity assay, SK-BR-3 breast cancer cells (ATCC, HTB-30) were seeded at 2000 cells per well in a 96-well cell culture plate and grown at 37° C. in a humidified chamber at 5% CO2. After 24 h, cells were incubated with serially diluted Trastuzumab (T), T-Exa-PABC-AA-C2-RKAA-PABC-G-Exa and T-Exa-PABC-ARA-C2-RKAA-PABC-G-Exa. Cell viability was measured using Cell Titre Glo (Promega) after 5 days of treatment. Percent viability was calculated as the luminescence values of treated cells divided by the values of untreated control cells. The EC50 was derived from the survival curve using the sigmoidal nonlinear regression analysis in GraphPad Prism.

### Results

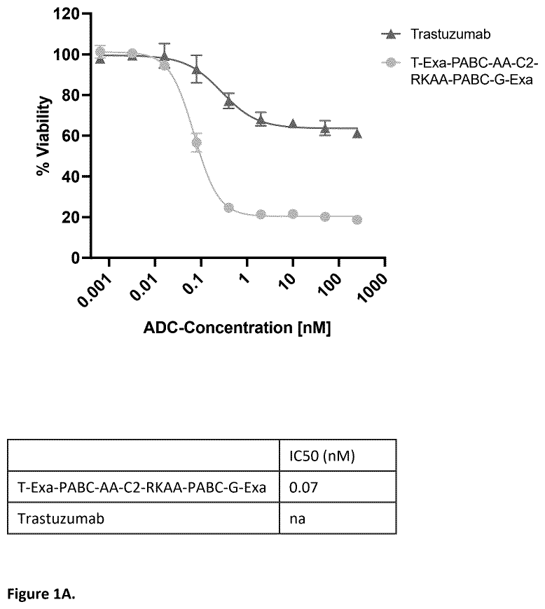

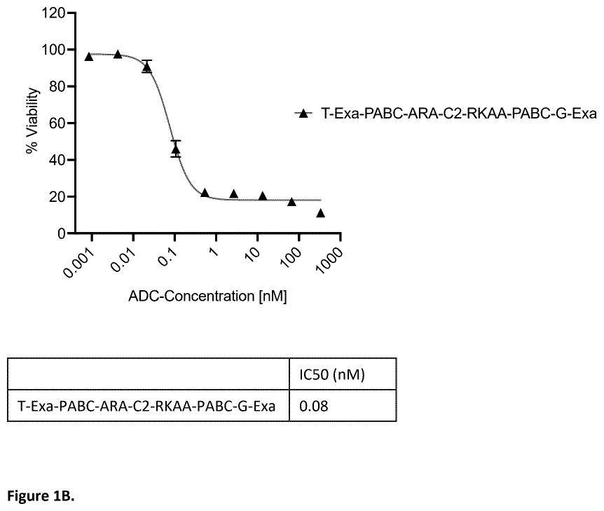

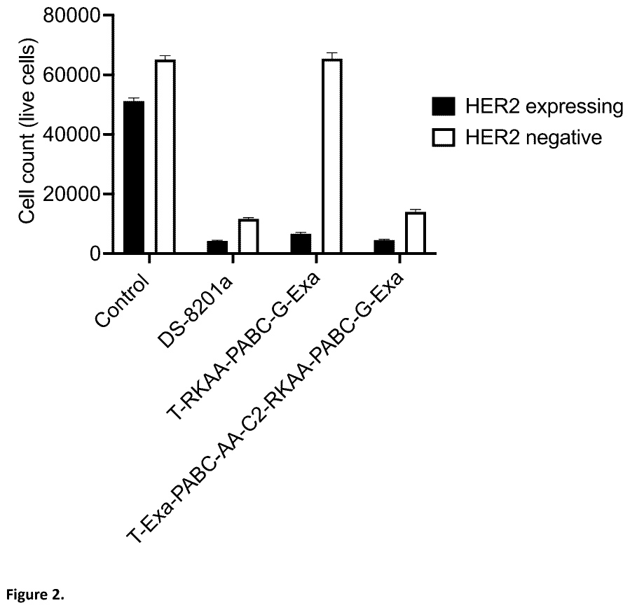

FIGS. 1A and 1B show that the ADCs of the invention T-Exa-PABC-AA-C2-RKAA-PABC-G-Exa and T-Exa-PABC-ARA-C2-RKAA-PABC-G-Exa (both DAR4) exert a very high cytotoxic activity with an IC50 of about 0.07 nM and 0.09 nM, respectively against HER-2 over-expressing SK-BR-3 breast cancer cells in vitro. Trastuzumab as control shows only limited cytotoxicity on this cell line.

### 3. Example 2: Bystander Killing Effects

### Method

In order to assess the bystander activity of our topoisomerase I inhibitors exatecan (“Exa”) and glycine-exatecan (“Gly-Exa”), a control ADC was generated with trastuzumab having just Gly-Exa as payload (T-RKAA-PABC-G-Exa) using the same method as described above, resulting in a drug-to-antibody ratio of 2 (DAR2).

For the bystander assay, HER-2-positive SK-BR-3 (ATCC HTB-30) breast cancer cells and HER-2-negative MDA-MB-468 (DSMZ ACC 738) cells were seeded into 6-well plates at a ratio of 1:5.5 and grown at 37° C. in a humidified chamber at 5% CO2. After 24 h, cells were treated with payload-adjusted concentrations of ADCs: 0.2 nM Enhertu® (bought at pharmacy), 0.4 nM T-Exa-PABC-AA-C2-RKAA-PABC-G-Exa and 0.8 nM T-RKAA-PABC-G-Exa, concentrations that are non-toxic to target-negative cells and toxic to target-positive cells. After 5 days, cells were harvested, stained with PE-HER2 (Biolegend, cat nr. 324405), fixed with Cyto-Fast fixing solution (Biolegend, cat nr. 426803) and measured in duplicates on the CytoFlex cytometer. Single cell counts for target-positive and target-negative populations were extracted per well with Flow Jo and plotted in GraphPad Prism.

### Results

In order to assess the bystander activity of our topoisomerase I inhibitors “Exa” and “Gly-Exa” a coculture cell killing assay was performed. DS8201a showed cell killing to both HER2 positive and HER2 negative cells, confirming the bystander activity of the payload of DS8201a, as described in the literature. Interestingly, our control ADC having just a “Gly-Exa” as payload (T-RKAA-PABC-G-Exa) was able to only kill HER2 positive cells but not co-culture HER2-negative cells, clearly showing that Gly-Exa is not able to exert bystander activity. The ADC of the invention T-Exa-PABC-AA-C2-RKAA-PABC-G-Exa, having Gly-Exa and Exa as payload, showed very potent bystander activity that was driven by the Exa payload only, because Gly-Exa is not able to exert bystander activity. In summary, the ADCs of the invention have two types of topoisomerase I inhibitors, Exa (bystander activity) and Gly-Exa (no bystander activity).

### 4. Example 3: Antibody-Drug Conjugates of the Invention Show Superior Anti-Tumor Effects In Vivo than DS-8201A

The anti-HER2 ADCs T-Exa-PABC-AA-C2-RKAA-PABC-G-Exa (FIG. 6), and T-Exa-PABC-ARA-C2-RKAA-PABC-G-Exa (FIG. 7) were investigated in vivo for tumor growth inhibition and were compared to the commercially available DS-8201a. Trastuzumab (Herceptin®) and DS-8201a (Enhertu®) were purchased, all other ADCs were produced in-house as described above.

### Method

1×10⁷ HER2-positive JIMT-1 human breast carcinoma cells were implanted s.c. with 50% matrigel into CB-17 SCID mice at Charles River Discovery Research Services North Carolina, US. Tumor dimensions and body weights were recorded as per study protocol. The tumor volume was calculated according to the formula volume=(width)²×length×0.5. When the average tumor size reached about 100-150 mm³, mice were allocated using a non-random stratification protocol into the treatment groups comprising five mice each. ADCs according to this invention, T-Exa-PABC-AA-C2-RKAA-PABC-G-Exa, and T-Exa-PABC-ARA-C2-RKAA-PABC-G-Exa at doses of 10 mg/kg, DS-8201a at 5 mg/kg (same payload dose as the ADCs of the invention) and Trastuzumab at 20 mg/kg by intravenous tail-vein injection on days 1 (day of randomization) and 8. Mice in the control group were injected with 25 mM Histidine.

### Results

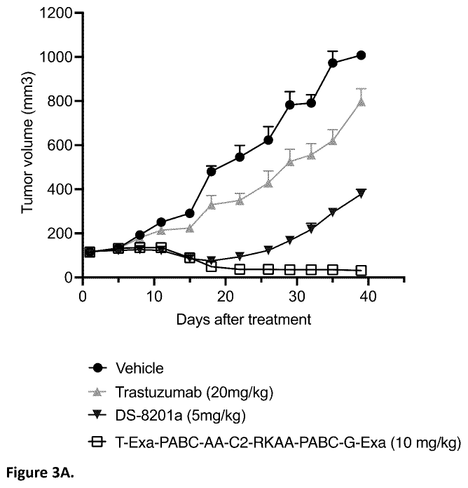

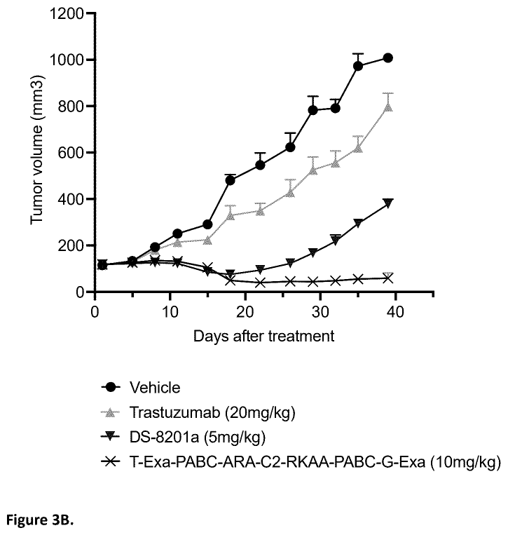

FIGS. 3A and 3B show the in vivo anti-tumor efficacy of the anti-HER2 ADC of this invention, T-Exa-PABC-AA-C2-RKAA-PABC-G-Exa (DAR4) and T-Exa-PABC-ARA-C2-RKAA-PABC-G-Exa (DAR4) as compared with DS-8201a (Enhertu®, DAR 8) in a HER2-overexpressing JIMT-1 tumor model. Specifically, the animals received two doses of 10 mg/kg of the corresponding ADC of the invention or 5 mg/kg of DS-8201s (animals received equal payload dose, because of the DAR difference).

Very surprisingly, T-Exa-PABC-AA-C2-RKAA-PABC-G-Exa and T-Exa-PABC-ARA-C2-RKAA-PABC-G-Exa demonstrated strong anti-tumoral activity. In contrast, at an equal payload dose, DS-8201a showed only transient anti-tumoral activity until about day 18, followed by rapid re-growth of the tumors. The trastuzumab control group (20 mg/kg) demonstrated only slight tumor growth retardation.

### 5. Example 4: Antibody-Drug Conjugates Comprising Two Different Types of Topoisomerase I Inhibitors and Another Payload with a Different Mechanism of Action

To demonstrate that the invention may also include antibody-drug conjugates comprising two different types of topoisomerase I inhibitors (Exa, Gly-Exa) and another payload with a different mechanism of action (MMAE), a linker-payload comprising three different drug types (Exa, Gly-Exa, and MMAE) was designed and conjugated to multiple different monoclonal antibodies resulting in DAR6 (2+2+2) ADCs comprising 2 Exa, 2 Gly-Exa, and 2 MMAE.

### Method

Multiple native, glycosylated monoclonal antibodies (ARA-27, ARA-01, ARA-29, m290) were conjugated to a linker-payload comprising two different types of topoisomerase I inhibitors and another payload with a different mechanism of action ((Exa-PABC-GR/Exa-G-PABC-GR)-CEA-C2-RK-PABC-MMAE) as follows: conjugation reactions were performed by mixing 5-10 mg/mL of native, glycosylated monoclonal antibodies ARA-27, ARA-01, ARA-29, m290, microbial transglutaminase at a concentration of 4-8 U/mg, and 4-5 molar equivalents of the indicated linker-payload, in 10-50 mM BisTris pH 6.0-6.8 for 6-24 hours at 37° C. in a rotating thermomixer. Conjugation efficiency was determined by LCMS, as described in the section general material and methods for the production of ADC.

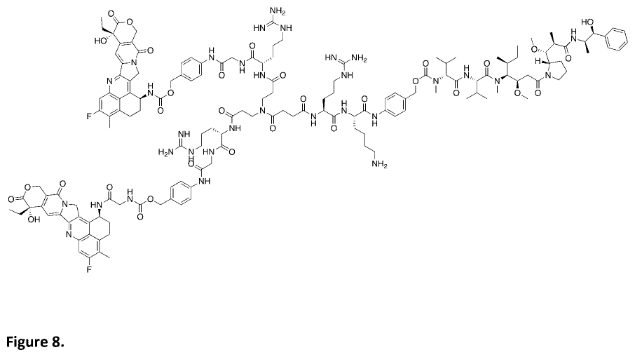

The chemical structure of the linker with three payloads (Exa, Gly-Exa and MMAE) is depicted in FIG. 8.

### Results

Linker-payload comprising two different types of topoisomerase I inhibitors and another payload with a different mechanism of action ((Exa-PABC-GR/Exa-G-PABC-GR)-CEA-C2-RK-PABC-MMAE) led to very high conjugation efficiencies (Table 1) on multiple native, fully glycosylated antibodies, resulting in DAR6 (2+2+2) ADCs comprising 2 Exa, 2 Gly-Exa and 2 MMAE.

TABLE 1
Conjugation efficiency (%) using Exa/Gly-Exa/MMAE linker-payload
to generate DAR6 (2 + 2 + 2) ADCs comprising 2 Exa,
2 Gly-Exa, and 2 MMAE based on different monoclonal antibodies

| Monoclonal antibody | Target | Conjugation efficiency (%) |
|---|---|---|
| ARA-01 | CD79b | 95% |
| (SEQ ID: 71, SEQ ID: 72) | | |
| ARA-27 | Nectin-4 | 96% |
| (SEQ ID: 94, SEQ ID: 95) | | |
| ARA-29 | NaPi2b | 99% |
| (SEQ ID: 100, SEQ ID: 101) | | |
| m290 | Nectin-4 | 99% |
| (SEQ ID: 96, SEQ ID: 97) | | |

### 6. Example 5: Anti-Nectin-4 Antibody Based ADC In Vivo Tumor Growth Inhibition

An anti-Nectin-4 antibody (in this example ARA-27, SEQ ID NO. 94 and 95) was conjugated with the peptide linker shown in FIG. 8 and the resulting ADC (ARC-121) was investigated in vivo for tumor growth inhibition in a SUM190PT (Nectin-4 positive, solid tumor) xenograft model and compared to the FDA approved ADC Padcev® (enfortumab-vedotin).

### Method

For SUM190PT xenografts, 2×10⁶ cells were injected into the mammary fatpad of CB17 SCID mice (Janvier). Tumor dimensions and body weights were recorded three times weekly. The tumor volume was calculated according to the formula volume=(width)²×length×0.5. When the average tumor size reached about 170 mm3, mice were allocated using a non-random stratification protocol to the respective treatment groups, N=5. ADCs were intravenously injected once on the day of randomization (d=0).

The conjugation of anti-Nectin-4 antibody (ARA-27) with peptide linker as shown in FIG. 8 was performed as described in general method section 1. Padcev® was bought at the Pharmacy.

0.5 mg/kg of ADC of the invention (ARC-121) and 0.5 mg/kg of Padcev® was injected intravenously on day 0. Mice in the vehicle control group were injected with formulation buffer. All mouse experiments were performed in accordance with Swiss guidelines and were approved by the Veterinarian Office of Zurich, Switzerland.

### Results

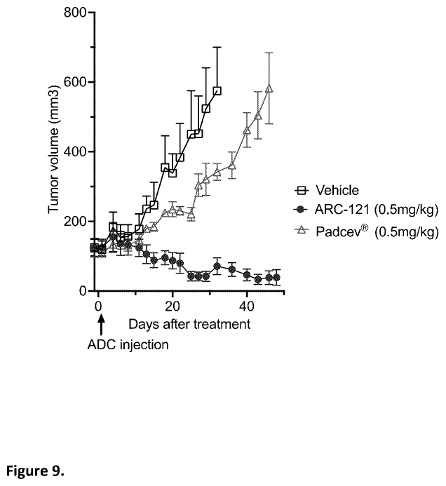

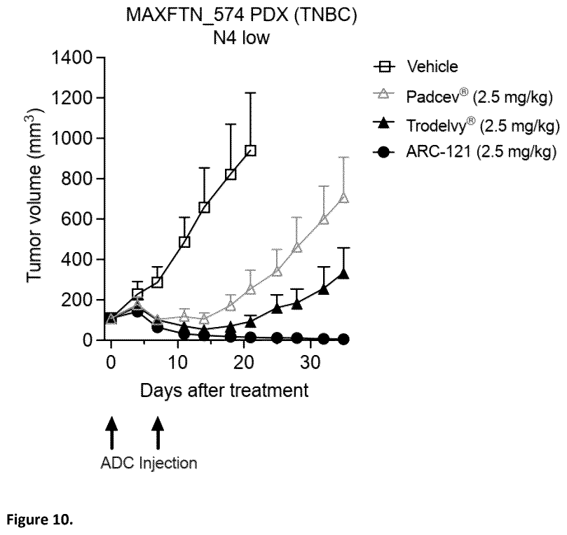

The ADC of the invention showed a significant anti-tumor effect at a dose of only 0.5 mg/kg, whereas Padcev® was hardly active at this dose (FIG. 9).

### 7. Example 6: Anti-Nectin-4 Antibody Based ADC In Vivo Tumor Growth Inhibition

An anti-Nectin-4 antibody (in this example ARA-27, SEQ ID NO. 94 and 95) was conjugated with the peptide linker shown in FIG. 8 and the resulting ADC (ARC-121) was investigated in vivo for tumor growth inhibition in the MAXFTN_574 (Nectin-4 positive, triple negative breast cancer TNBC) patient-derived xenograft (PDX) model and compared to the FDA approved ADCs Trodelvy® (approved for the treatment of TNBC) and anti-Nectin-4 ADC Padcev®.

### Method

Female NMRI nu/nu mice (Charles River) were unilaterally implanted subcutaneously with MAXFTN_574 tumor tissue passaged from donor-animals. Once tumors reached a volume of approx. 100 mm³. Mice were randomized into the different treatment arms of 5 animals each. Tumor volume and body weight were measured bi-weekly. 2.5 mg/kg of ADC of this invention 2.5 mg/kg of Trodelvy® or 2.5 mg/kg of Padcev© were injected intravenously on day 0 and day 7. Mice in the control group were injected with formulation buffer.

ADC was produced as described in general method section 1. Trodelvy® and Padcev® were provided by Charles River, studies were performed at Charles River Laboratories Germany GmbH, Freiburg.

### Results

The ADC of this invention ARC-121 showed a better anti-tumoral effect than FDA approved ADCs (FIG. 10).

1. An antibody-drug conjugate (ADC) having the formula A-L, wherein A is an antibody or an antibody fragment and wherein L is a linker, said linker comprising:
as a first payload a topoisomerase I inhibitor which is cell-permeable; and
as a second payload a topoisomerase I inhibitor which is not cell-permeable.

2. The ADC according to claim 1, wherein the first or second payload is a camptothecin cytotoxic molecule.

3-8. (canceled)

9. The ADC according to claim 1, wherein the linker consists of or comprises the following structure: 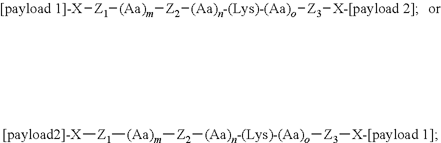
wherein
[payload 1] is said first payload;
[payload 2] is said second payload;
(Aa) is any amino acid residue;
m, n and o may be integers ranging from 0 to 10;
(Lys) is a lysine residue, a lysine mimetic or a lysine derivative;
Z₁₋₃ is either absent or a spacer comprising an alkyl or a heteroalkyl group; and
X is either absent or a self-immolative group.

10. (canceled)

11. The ADC according to claim 9, wherein Z₂ is a dicarboxylic acid linking the N-terminal end of (Aa)ₘ to the N-terminal end of (Aa)ₙ or (Lys); and wherein one payload is linked to the C-terminal end of (Aa)ₘ and the other payload is linked to the C-terminal end of (Lys) or (Aa)ₒ, or vice versa.

12. (canceled)

13. The ADC according to claim 9, wherein (Aa)ₙ-(Lys)-(Aa)ₒ comprises the sequence motif Arg-Lys (RK), RKAA, or His-Lys (HK) (in N->C direction).

14. (canceled)

15. The ADC according to claim 1, wherein the linker comprises or consists of the following structure:

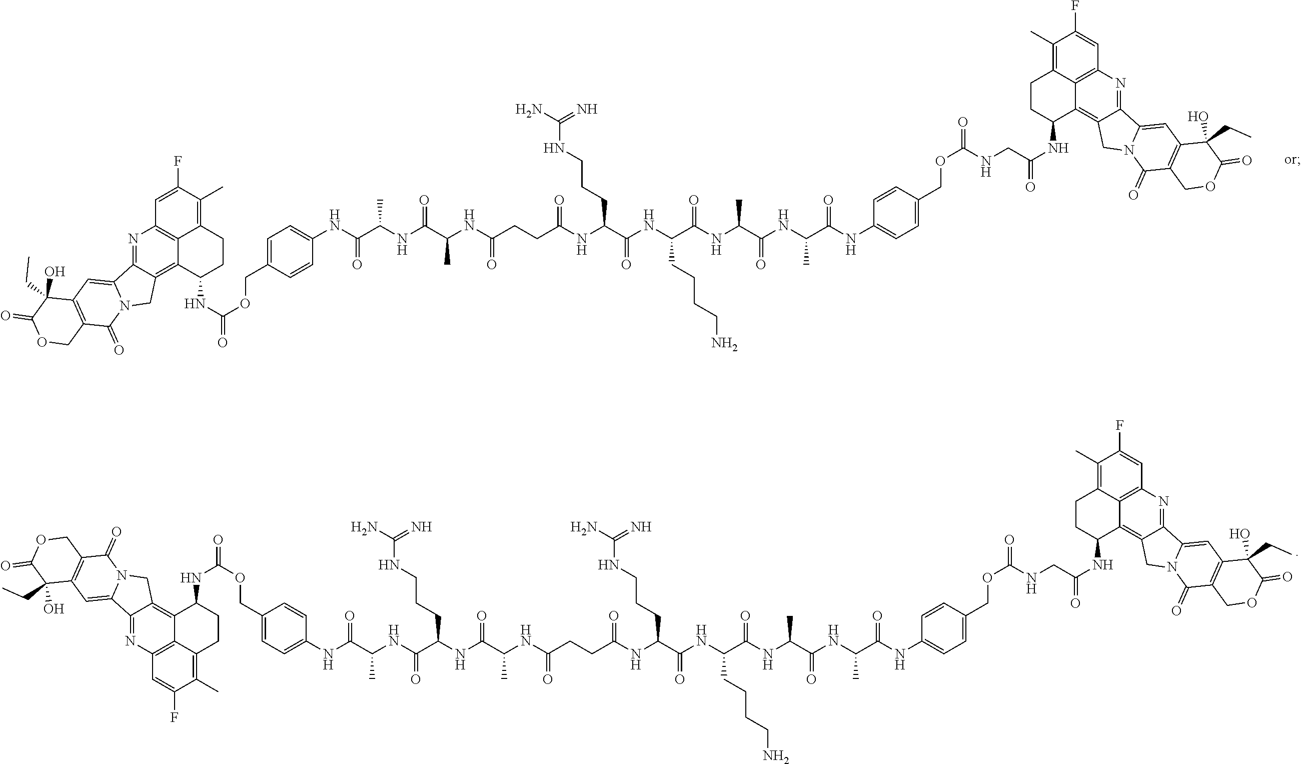

16. (canceled)

17. The ADC according to claim 1, wherein said ADC consists of two first payloads and two second payloads (drug-to-antibody ratio of 4 “DAR4”).

18-27. (canceled)

28. The ADC according to claim 7, wherein the antibody is an IgG antibody and the linker is conjugated to the antibody via an isopeptide bond formed between the glutamine residue Q295 (EU numbering) of the C_{H}2 domain of the IgG antibody.

29. The ADC according to claim 1, wherein the antibody is an IgG antibody glycosylated at residue N297 (EU numbering).

30. (canceled)

31. A pharmaceutical composition comprising the ADC according to claim 1 and at least one pharmaceutically acceptable ingredient.

32. A method for treating a patient suffering from, being at risk of developing, and/or being diagnosed for a neoplastic disease, the method comprising administering to the patient an antibody-drug conjugate (ADO) having the formula A-L, wherein A is an antibody or an antibody fragment and wherein L is a linker, said linker comprising:
as a first payload a topoisomerase I inhibitor which is cell-permeable; and
as a second payload a topoisomerase I inhibitor which is not cell-permeable.

33. The method of claim 32,
wherein the antibody-payload conjugate comprises Trastuzumab and wherein the neoplastic disease is a HER2-positive cancer;
wherein the antibody-payload conjugate comprises Polatuzumab and wherein the neoplastic disease is a B-cell associated cancer; or
wherein the antibody-payload conjugate comprises Enfortumab or an Enfortumab variant and wherein the neoplastic disease is a Nectin-4 positive cancer.

34. The method of claim 1,
wherein the antibody-payload conjugate comprises an antibody targeting NaPi2b, wherein the antibody targeting NaPi2b is Upifitamab with a heavy chain as set forth in SEQ ID NO:98 and a light chain as set forth in SEQ ID NO:99, or with a heavy chain as set forth in SEQ ID NO:100 and a light chain as set forth in SEQ ID NO:101, and wherein the neoplastic disease is a NaPi2b positive cancer, in particular NaPi2b positive lung or ovarian cancers.

35. (canceled)

36. A linker comprising a first and second payload, wherein the first payload is a topoisomerase I inhibitor which is cell-permeable; and
wherein the second payload a topoisomerase I inhibitor which is not cell-permeable.

37-40. (canceled)

41. The linker according to claim 36, wherein wherein linker is a peptide linker, the first payload is linked to the N-terminus of the peptide linker and the second payload is linked to the C-terminus of the peptide linker, or vice versa.

42. (canceled)

43. (canceled)

44. The linker according to claim 36, wherein the linker consists of or comprises the following structure: 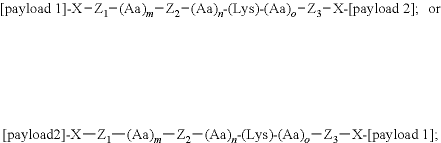
wherein
[payload 1] is said first payload;
[payload 2] is said second payload;
(Aa) is any amino acid residue;
m, n and o may be integers ranging from 0 to 10;
(Lys) is a lysine residue, a lysine mimetic or a lysine derivative;
Z₁-3 is either absent or a spacer comprising an alkyl or a heteroalkyl group; and
X is either absent or a self-immolative group.

45. (canceled)

46. The linker according to claim 44, wherein Z₂ is a dicarboxylic acid linking the N-terminal end of (Aa)ₘ to the N-terminal end of (Aa)ₙ or (Lys); and wherein one payload is linked to the C-terminal end of (Aa)ₘ and the other payload is linked to the C-terminal end of (Lys) or (Aa)ₒ, or vice versa.

47. (canceled)

48. The linker according to claim 44, wherein (Aa)ₙ-(Lys)-(Aa)ₒ comprises the sequence motif Arg-Lys (RK) RKAA or His-Lys (HK) (in N->C direction).

49. (canceled)

50. The linker according to claim 36, wherein the linker comprises or consists of the following structure:

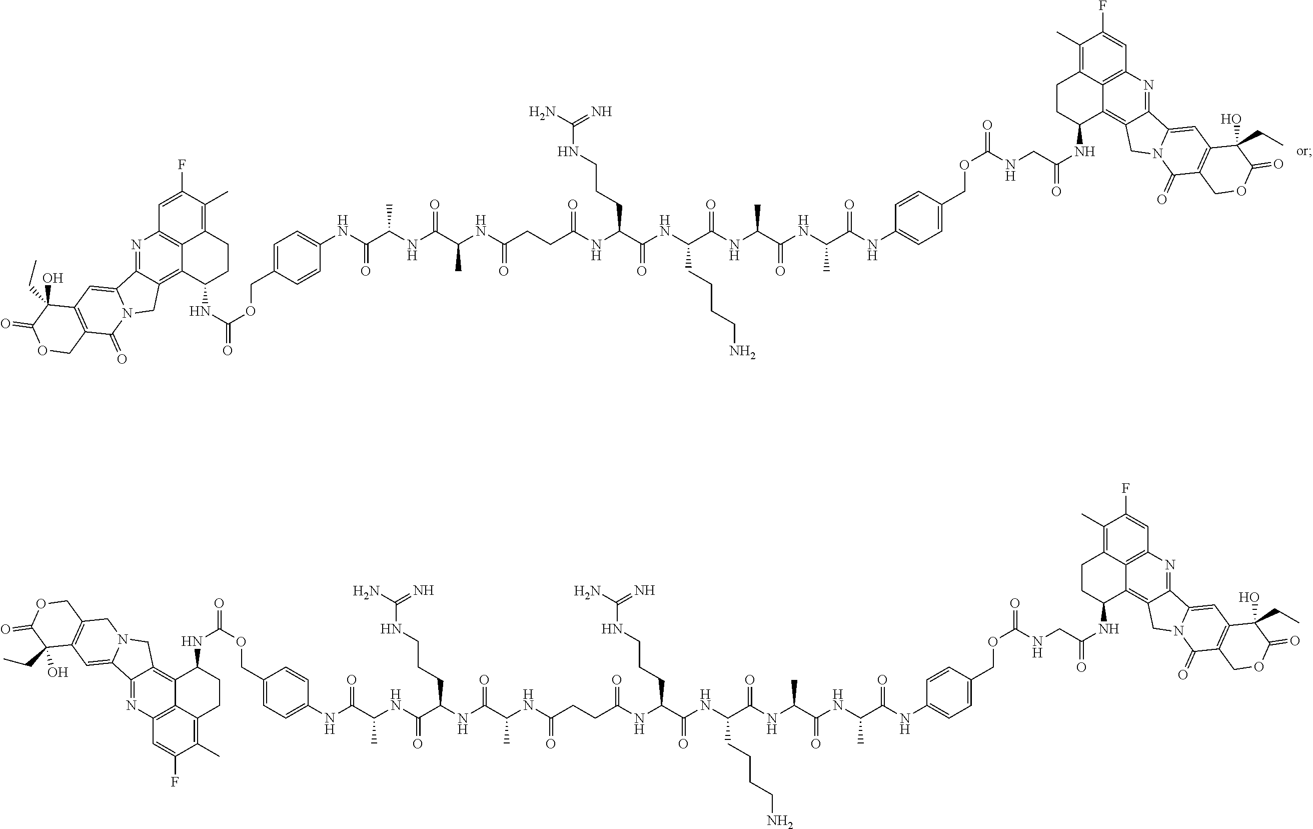

51-59. (canceled)

60. A method for the preparation of an antibody-drug conjugate comprising a step of conjugating a peptide linker to an antibody, said linker comprising:
as a first payload a topoisomerase I inhibitor which is cell-permeable; and
as a second payload a topoisomerase I inhibitor which is not cell-permeable.

61. The method of claim 60, the method comprising a) mixing the antibody, the peptide linker and a transglutaminase (TG) within a fluid, thereby conjugating the petpide linker to the antibody in one step under the catalyzing effect of the TG, and b) extracting the conjugate obtained in step a) from the fluid.

62. The method according to claim 61, wherein the peptide linker is conjugated to a glutamine residue comprised in the antibody via a primary amine comprised in an amino acid residue of the peptide linker.

63. The method according to claim 61, wherein the antibody is an IgG antibody and the peptide linker is conjugated to a glutamine residue comprised in an Fc domain of the IgG antibody.

64. The method according to claim 62, wherein the glutamine residue to which the peptide linker is conjugated is glutamine residue Q295 (EU numbering) of the C_{H}2 domain of an IgG antibody.

65. (canceled)

66. The method according to claim 62, wherein the IgG antibody is glycosylated at residue N297 (EU numbering) of the C_{H}2 domain.

67. (canceled)

68. (canceled)

69. The method according to claim 61, wherein the transglutaminase is a microbial transglutaminase (MTG) is derived from a Streptomyces species.

70. The ADC of claim 1,
wherein the first and second payload is an exatecan;
wherein the second payload has a glycine residue linked to said second payload;
wherein said ADC consists of two first payloads and two second payloads (drug-to-antibody ratio of 4 “DAR4”); and
wherein said antibody is an IgG1 antibody.

71. The ADC according to claim 70, wherein the antibody is Trastuzumab or Upifitamab.

72. (canceled)

73. The ADC according to claim 15, wherein the antibody is m290.

74. The ADC according to claim 34, wherein the linker comprises or consists of the following structure: 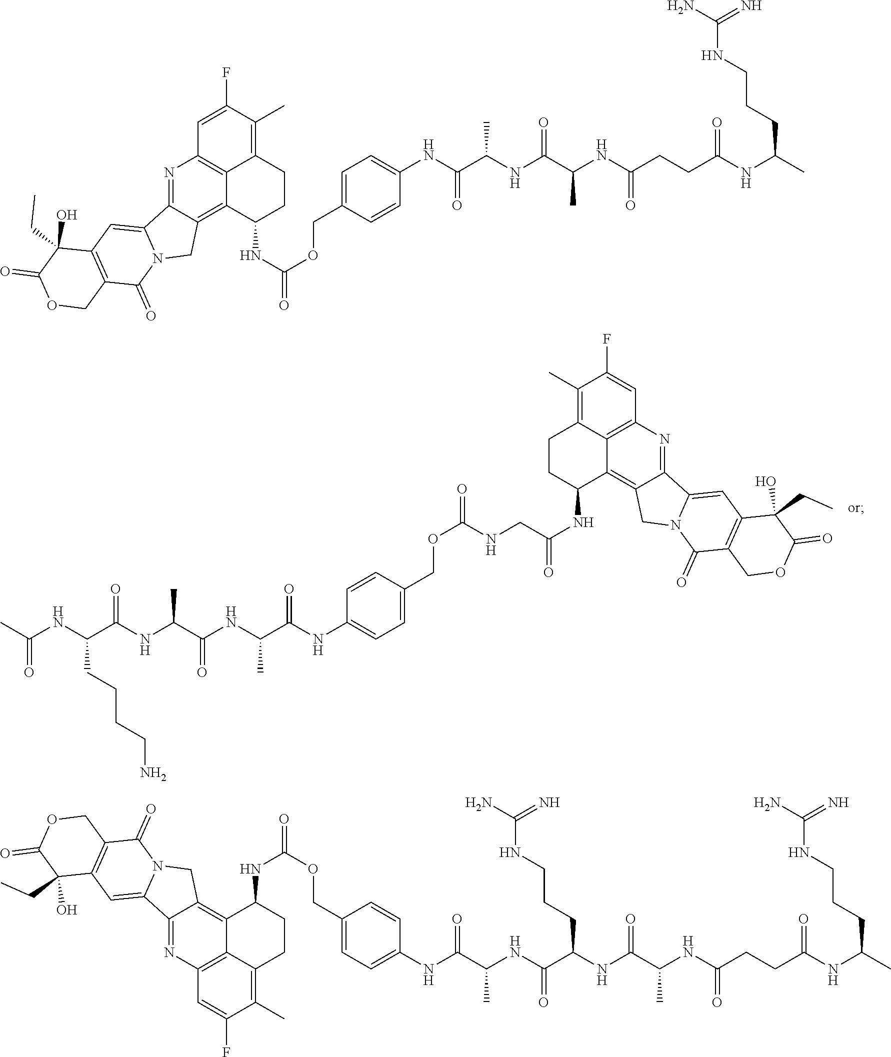 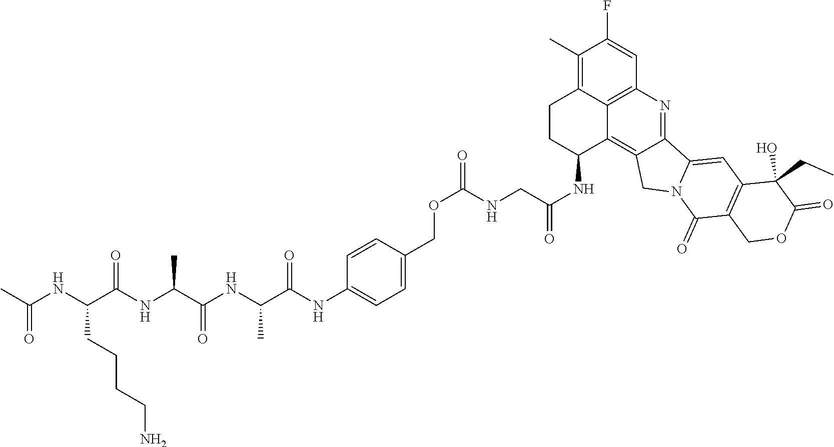 [SMILES: CC[C@@]1(O)C(=O)OCc2c1cc1n(c2=O)Cc2c-1nc1cc(F)c(C)c3c1c2[C@@H](NC(=O)CNC(=O)OCc1ccc(NC(=O)[C@H](C)NC(=O)[C@H](C)NC(=O)[C@H](CCCCN)NC(C)=O)cc1)CC3]
